(19) United States
(12) Patent Application Publication
(10) Pub. No.: US 2014/0066462 A1
(43) Pub. Date: Mar. 6, 2014

(54) TREATMENT OF POLYCYSTIC DISEASE

(71) Applicants: Intellikine LLC, La Jolla, CA (US); The Regents of the University of California, Oakland, CA (US)

(72) Inventors: David Pearce, Belmont, CA (US); Yi Liu, San Diego, CA (US); Michael Martin, Carlsbad, CA (US); Christian Rommel, La Jolla, CA (US); Pingda Ren, San Diego, CA (US); Troy Edward Wilson, San Marino, CA (US)

(73) Assignees: Intellikine LLC, La Jolla, CA (US); The Regents of the University of California, Oakland, CA (US)

(21) Appl. No.: 14/073,090

(22) Filed: Nov. 6, 2013

Related U.S. Application Data
(63) Continuation of application No. PCT/US12/36841, filed on May 7, 2012.
(60) Provisional application No. 61/483,630, filed on May 6, 2011.

(51) Int. Cl. A61K 31/52; A61K 31/437

(52) U.S. Cl. CPC A61K 31/52; A61K 31/437
USPC 514/263.2; 514/303

(57) ABSTRACT

The present invention provides methods of treating polycystic disorders. In particular, methods include the use of inhibitors targeting certain protein kinases, such as mTOR, to treat polycystic disease.

### CROSS-REFERENCE TO RELATED APPLICATIONS

This application is a continuation of PCT Application No. PCT/US2012/036841, filed May 7, 2012, which claims the benefit of U.S. Provisional Application No. 61/483,630, filed May 6, 2011, which are incorporated herein by reference in their entirety and for all purposes.

### BACKGROUND OF THE INVENTION

Human autosomal polycystic diseases can be classified into at least three categories that are associated with mutations in at least six different genes. These three categories are autosomal dominant polycystic kidney disease (ADPKD) caused by mutated PKD1 or PKD2 gene; autosomal recessive polycystic kidney disease (ARPKD) caused by mutated PKHD1 gene; and autosomal dominant polycystic liver disease (ADPLD) caused by mutated PLD1, PLD2, PLD3 gene. Of these cilial diseases, ADPKD represents the largest public health burden. ADPKD affects between 1 in 500 and 1 in 1000 live births worldwide and is the leading genetic cause of end stage renal failure. Mutations in PKD1 (encoding polycystin-1) account for approximately 85% of all cases of ADPKD, while the remaining almost all attributed to mutations in PKD2 (encoding polycystin-2) (Chapin et al., 2010, JCB 4, 701-710.). The mutational mechanism for cyst formation in ADPKD involves somatic acquisition of mutations in the normal copy of the respective genes that lead to cyst formation. ARPKD accounts for less than 10% of cases of PKD, develops in utero, and leads to small cyst formation in the collecting tubules of the kidney.

Polycystic kidney disease (PKD) disease progression is characterized by the consequent formation and growth of fluid filled cysts derived from tubules throughout the kidney, and potentially elsewhere in the body. The cellular pathogenesis of these changes is related to the inability of tubule epithelium to regulate calcium signals, which results in a loss of the fully differentiated state, increased proliferation, net fluid secretion and the formation of fluid-filled cysts in the kidney. Normal cell function and nephron structure is under the control of the mechano- and chemosensory function of primary cilia. Polycystin-1 (PC-1; also referred to as PKD1) and polycystin-2 (PC-2; also referred to as PKD2) co-localize to the primary cilia of the kidney tubule cells and bile duct cells. Polycystin-2 is a cation channel permeant to calcium (Koulen, et al., 2002, Nat Cell Biol 4, 191-197). In response to laminar flow shear stress forces, the cell's primary cilium act as mechanosensors, bending and allowing calcium to enters the cell. In PKD patients, mutations to PKD1 and PKD2 lead to disruption this regulated process. As a consequence of cyst formation, PKD patients exhibit enlarged kidneys. Several treatments are available for treating the symptoms of PKD, e.g. methods of directly draining cysts. However, there are no effective therapeutic agents for treating PKD. Currently the only therapeutic intervention available to patients who develop kidney failure from polycystic kidney disease is renal replacement by either dialysis or transplantation. As such, there exists a pressing need for alternative treatments for PKD patients.

The mammalian target of rapamycin (mTOR) is a serine-threonine kinase related to the lipid kinases of the phosphatidylinositol 3 kinase (PI3K) family. mTOR has been implicated in a wide range of biological processes including cell growth/proliferation, cell motility and survival. Dysregulation of the mTOR pathway has been reported in various types of cancer. mTOR is a multifunctional kinase that integrates growth factor and nutrient signals to regulate protein translation, nutrient uptake, autophagy and mitochondrial function. mTOR exists in two complexes, mTORC1 and mTORC2. mTORC1 contains the raptor subunit and mTORC2 contains rictor. These complexes are differentially regulated, and have distinct substrate specificities and rapamycin sensitivity. For example, mTORC1 phosphorylates S6 kinase (S6K) and 4EBP1 (eIF4E-binding protein 1, also known as also known as EIF4EBP1), promoting increased translation and ribosome biogenesis to facilitate cell growth and cell cycle progression. S6K also acts in a feedback pathway to attenuate PI3K/Akt activation. mTORC2 is generally insensitive to rapamycin. mTORC2 is thought to modulate growth factor signaling by phosphorylating the C-terminal hydrophobic motif of some AGC kinases such as Akt. In many cellular contexts, mTORC2 is required for phosphorylation of the S473 site of Akt.

### SUMMARY OF THE INVENTION

The present invention provides for the use of compounds (e.g. selective mTOR inhibitors) for treating autosomal polycystic disorders, including PKD.

In one aspect, the invention provides a method of treating polycystic kidney disease (PKD) in a subject in need thereof, comprising administering to the subject a therapeutically effective amount of a compound of Formula (I):

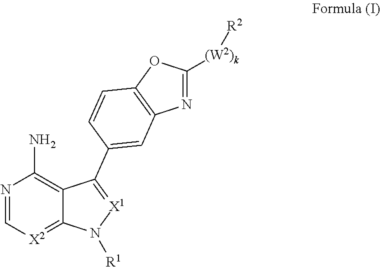

wherein:

### X¹ is N or C-E¹;

### X² is N or CH;

E¹ is —(W¹)ⱼ—R⁴;

### W¹ is —O—, —NR^{7A}—, —S(O)₀₋₂—, —C(O)—, —C(O)N(R^{7A})—, —N(R^{7A})C(O)—, or —N(R^{7A})C(O)N(R^{8A})—;

### W² is —O—, —NR⁷—, —S(O)₀₋₂—, —C(O)—, —C(O)N(R⁷)—, —N(R⁷)C(O)—, or —N(R⁷)C(O)N(R⁸)—;

j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂ C₁₋₁₀alkylaryl, —S(O)₀₋₂aryl, —SO₂N(aryl), —SO₂N(C₁₋₁₀alkyl)(C_{—10}alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R⁸, R^{7A} and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C(═O)NR³¹R³², —C(═O)NR³⁴R³⁵, —SO₂NR³⁴R³⁵, —SO₂NR³¹R³², NR³¹R³², or —NR³⁴R³⁵.

In one embodiment, the compound (e.g. mTOR inhibitor) selectively inhibits both mTORC1 and mTORC2 activity. In a further embodiment, the compound (e.g. mTOR inhibitor) selectively inhibits both mTORC1 and mTORC2 activity relative to one or more type I phosphatidylinositol 3-kinases (PI3-kinase) as ascertained in a cell-based assay or an in vitro kinase assay, wherein the one or more type I PI3-kinase is selected from the group consisting of PI3-kinase α, PI3-kinase β, PI3-kinase γ, and PI3-kinase δ. In another embodiment, the subject is a mammal. In another embodiment, the compound (e.g. mTOR inhibitor) inhibits mTOR activity with an IC₅₀ value of about 100 nM or less as ascertained in an in vitro kinase assay. In another embodiment, the compound (e.g. mTOR inhibitor) inhibits mTOR activity with an IC₅₀ value of about 10 nM or less as ascertained in an in vitro kinase assay. In another embodiment, said administration of the compound (e.g. mTOR inhibitor) decreases kidney size, decreases cyst volume, and/or increases glomeruli number in a subject. In another embodiment, the compound (e.g. mTOR inhibitor) is administered parenterally, orally, intraperitoneally, intravenously, intraarterially, transdermally, intramuscularly, liposomally, via local delivery by catheter or stent, subcutaneously, intraadiposally, or intrathecally. In another embodiment, the treatment reduces kidney mass in the subject by at least 10%. In another embodiment, the treatment reduces kidney mass in the subject by at least 50%. In another embodiment, the treatment reduces normalized kidney mass in the subject by at least 10%. In another embodiment, the treatment reduces normalized kidney mass in the subject by at least 30%. In another embodiment, the administration of the compound (e.g. mTOR inhibitor) is prior to, concurrent with, or after administration of another treatment to the subject.

In another aspect, a method is provided for inhibiting cyst formation in a subject at risk for developing PKD, comprising contacting cyst cells with a compound of Formula (I) in an amount sufficient to inhibit growth of cyst cells:

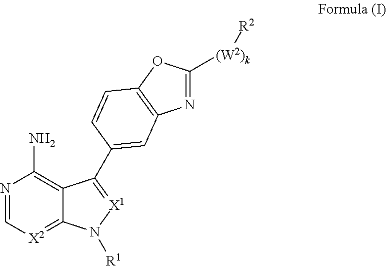

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, or —N(R^{7A})C(O)N(R^{8A})—; W² is —O—, —NR⁷—, —S(O)₀₋₂—, —C(O)—, —C(O)N(R⁷)—, —N(R⁷)C(O)—, or —N(R⁷)C(O)N(R⁸)—;
j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C_{—10}alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

In one embodiment, the method further comprises reducing cyst formation in an organ other than kidney.

In yet another embodiment, the present invention provides a method that comprises the steps of: (a) evaluating whether a subject is susceptible to PKD, wherein said evaluation comprises testing for (i) the presence of a biomarker correlated with PKD in said subject and/or (ii) the presence of multiple kidney cysts; and (b) administering to the subject being tested for (a)(i) and/or (a)(ii) a pharmaceutical composition comprising an effective amount of a compound of Formula (I):

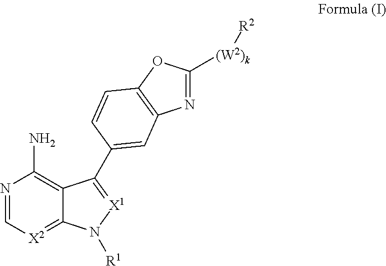

wherein:

### X¹ is N or C-E¹;

### X² is N or CH;

E¹ is —(W¹)ⱼ—R⁴;

### W¹ is —O—, —NR^{7A}—, —S(O)₀₋₂—, —C(O)—, —C(O)N(R^{7A})—, —N(R^{7A})C(O)—, or —N(R^{7A})C(O)N(R^{8A})—;

### W² is —O—, —NR⁷—, —S(O)₀₋₂—, —C(O)—, —C(O)N(R⁷)—, —N(R⁷)C(O)—, or —N(R⁷)C(O)N(R⁸)—;

j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C_{—10}alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

In one embodiment, the biomarker comprises mutations in PKD-1 or PKD-2 genes.

In another aspect, a method of treating a polycystic disease in a subject in need thereof is provided. The method including administering to the subject a therapeutically effective amount of a compound of Formula (I):

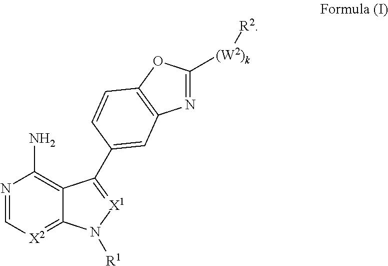

X¹ is N or C-E¹. X² is N or CH. E¹ is —(W¹)ⱼ—R⁴. W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—. W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—. The symbol j is 0 or 1. The symbol k is 0 or 1. R¹ is —H, -aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl, each of which, except for —H, is unsubstituted or is substituted by one or more independent R³. R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl (e.g. bicyclic aryl, unsubstituted aryl, or substituted monocyclic aryl), heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl moiety, wherein each of said moieties is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —O-aryl or —SC(═O)NR³¹R³². R³ and R⁴ are independently hydrogen, halogen, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl (e.g. bicyclic aryl, unsubstituted aryl, or substituted monocyclic aryl), heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl moiety, wherein each of said moieties is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³². Each of R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or C₁₋₁₀alkyl, C₂₋₁₀alkenyl, C₂₋₁₀alkynyl, C₃₋₈cycloalkyl, heteroalkyl, aryl, heteroaryl, or heterocyclyl moiety, wherein each of said moieties is unsubstituted or is substituted with one or more C₁₋₁₀alkyl, C₂₋₁₀alkenyl, C₂₋₁₀alkynyl, C₃₋₁₀cycloalkyl, heteroalkyl, aryl, heteroaryl, heterocyclyl substituent, wherein each of said substituents is unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂ NH(C₁₋₁₀alkyl), —COOH, or —SO₂NR³⁴R³⁵. R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵, and wherein said 3-10 membered saturated or unsaturated ring independently contains 0, 1, or 2 more heteroatoms in addition to the nitrogen atom. Each of R⁷, R^{7A}, R⁸, and R^{8A} is independently hydrogen, C₁₋₁₀alkyl, C₂₋₁₀alkenyl, C₂₋₁₀alkynyl, aryl, heteroalkyl, heteroaryl, heterocyclyl or C₃₋₁₀cycloalkyl, each of which except for hydrogen is unsubstituted or is substituted by one or more independent R⁶ substituents. R⁶ is independently halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³²; or C₁₋₁₀alkyl, C₂₋₁₀alkenyl, C₂₋₁₀alkynyl, C₃₋₈cycloalkyl, heteroalkyl, aryl, heteroaryl, heterocyclyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, oxo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

In another aspect, a method of treating a polycystic disease in a subject in need thereof is provided. The method including administering to the subject a therapeutically effective amount of a compound of Formula (I):

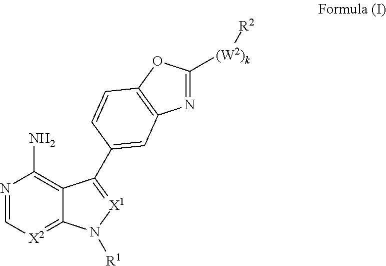

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—. W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—. The symbol j is 0 or 1. The symbol k is 0 or 1. R¹ is hydrogen, R³-substituted or unsubstituted C₁₋₁₀alkyl, R³-substituted or unsubstituted C₂₋₁₀alkenyl, R³-substituted or unsubstituted C₂₋₁₀alkynyl, R³-substituted or unsubstituted C₃₋₈cycloalkyl, R³-substituted or unsubstituted C₃₋₈cycloalkenyl, R³-substituted or unsubstituted C₃₋₈cycloalkynyl, R³-substituted or unsubstituted heteroalkyl, R³-substituted or unsubstituted heteroalkenyl, R³-substituted or unsubstituted heteroalkynyl, R³-substituted or unsubstituted heterocyclyl, R³-substituted or unsubstituted aryl, R³-substituted or unsubstituted heteroaryl; wherein each R³-substituted R¹ is independently substituted with one or more R³. R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl. Each substituted R² is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³² substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl). R³ and R⁴ are independently is hydrogen, oxo, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹R³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl. Each substituted R³ or R⁴ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, C(═O)NR³¹R³², C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl). R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl. Each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵. R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵. Each R⁷, R^{7A}, R⁸, and R^{8A} is independently hydrogen, R⁶-substituted or unsubstituted C₁₋₁₀alkyl, R⁶-substituted or unsubstituted C₂₋₁₀alkenyl, R⁶-substituted or unsubstituted C₂₋₁₀alkynyl, R⁶-substituted or unsubstituted C₃₋₈cycloalkyl, R⁶-substituted or unsubstituted C₃₋₈cycloalkenyl, R⁶-substituted or unsubstituted C₃₋₈cycloalkynyl, R⁶-substituted or unsubstituted heteroalkyl, R⁶-substituted or unsubstituted heteroalkenyl, R⁶-substituted or unsubstituted heteroalkynyl, R⁶-substituted or unsubstituted heterocyclyl, R⁶-substituted or unsubstituted aryl, R⁶-substituted or unsubstituted heteroaryl; wherein each R⁶-substituted R⁷, R^{7A}, R⁸ and R^{8A} is independently substituted with one or more R⁶. R⁶ is independently halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl. Each substituted R⁶ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the invention are set forth with particularity in the appended claims. A better understanding of features and advantages of the present invention will be obtained by reference to the following detailed description that sets forth illustrative embodiments, in which the principles of the invention are utilized, and the accompanying drawings of which:

FIG. 1 illustrates a schematic of the mTORC1/2 pathway.

FIG. 2 illustrates the in vivo effect of Compound A on AKT phosphorylation (p-AKT). P11 PKD(V/V) (“V/V”) mice were treated with Compound A (0.5 mg/kg; “+”) or vehicle (M-”) p.o. Animals were sacrificed 2 hours later and kidney crude lysates were prepared and subjected to Western blot. A) Akt-S473 and T308 phosphorylation were elevated in mutant mice. B) S473 and T308 phosphorylation was moderately attenuated by Compound A. C) Total Akt was increased in mutant mice, but is unaffected by treatment with Compound A. These findings were consistent in several experiments.

FIG. 3 illustrates the effect of Compound A on 4EBP1 phosphorylation (p-4EBP1). Western blots from experiments illustrated in FIG. 2 were stripped and restained with antibodies to p4E-BP1 and total 4E-BP1. A) p4E-BPI is markedly elevated in mutant mice, and markedly inhibited by treatment with Compound A. B) Baseline phosphorylation and expression of 4E-BP1 in wt mice was low, but also markedly attenuated by Compound A.

FIG. 4 illustrates the effect of Compound A on S6 ribosomal protein phosphorylation (p-S6). Western blots from experiments illustrated in FIG. 3 were stripped and restained with antibodies to S6-RP and pS6-RP. A) pS6 was markedly elevated in mutant mice, and markedly inhibited by Compound A. Baseline phosphorylation of S6 was low in wt and mutant mice, but also markedly attenuated by Compound A. B) S6 expression was relatively unaffected by mutation or Compound A.

FIG. 5 illustrates the effect of Compound A on kidney size in V/V mice. PKD (V/V) mice were treated from P5-P11 with either vehicle or Compound A. Compound A dosing was 0.25 mg/kg on P5/P6, then 0.25 mg/kg bid on P7/P8, then 0.5 mg/kg bid on P9-11. Animals were sacrificed 2 hours after fast dose; kidneys were weighed, and one kidney was subjected to western blot and one to sectioning for histology. A) Body mass in mutant and in Het/WT mice was decreased by treatment with Compound A (p<0.05). B) Average kidney mass was significantly lower in Compound A-treated as compared to vehicle treated mutants (p=0.007). In contrast, kidney mass was not significantly changed by Compound A in Het/WT (p=0.22). C) Normalized kidney mass (combined kidney weight/body weight) was significantly lower in Compound A-treated compared with vehicle-treated mutants (p=0.01). Compound A had no significant effect on normalized kidney mass in Het/WT mice (p=0.5).

FIG. 6 illustrates the effect of Compound A on kidney histology in V/V mice. PKD (V/V) mice were treated from P5-P11 with either vehicle (A) or Compound A (B). Sagittal sections of the left kidney were stained with H&E at 4× magnification. Cyst volume was lower and parenchyma was increased in Compound A-treated mice.

FIG. 7 illustrates the sections from FIG. 6 captured at 10× magnification.

FIG. 8 illustrates the sections from FIG. 6 captured at 20× magnification. The number of glomeruli is increased in the Compound A treated slide.

FIG. 9 illustrates the sections from FIG. 6 captured at 40× magnification. The glomeruli appear normal in the Compound A treated kidney as compared to the untreated kidney.

### DETAILED DESCRIPTION OF THE INVENTION

### I. Definitions

The terms and “RAP” and “rapamycin”, refer to the same compound and are interchangeable.

The term “IP” or “i.p.” as used herein refers to intraperitneal administration.

The term “p.o.” as used herein refers to oral administration or oral lavage.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “antagonist” as used herein refers to a molecule having the ability to inhibit a biological function of a target polypeptide. Accordingly, the term “antagonist” is defined in the context of the biological role of the target polypeptide. While preferred antagonists herein specifically interact with (e.g. bind to) the target, molecules that inhibit a biological activity of the target polypeptide by interacting with other members of the signal transduction pathway of which the target polypeptide is a member are also specifically included within this definition. A preferred biological activity inhibited by an antagonist is associated with the development, growth, or spread of a cyst. Antagonists, as defined herein, without limitation, include antibodies and immunoglobulin variants, peptides, peptidomimetics, non-peptide small molecules, antisense molecules, and oligonucleotide decoys.

The term “agonist” as used herein refers to a molecule having the ability to initiate or enhance a biological function of a target polypeptide. Accordingly, the term “agonist” is defined in the context of the biological role of the target polypeptide. While preferred agonists herein specifically interact with (e.g. bind to) the target, molecules that increase a biological activity of the target polypeptide by interacting with other members of the signal transduction pathway of which the target polypeptide is a member are also specifically included within this definition. A preferred biological activity increased by an agonist is associated with the prevention or inhibition of the development, growth, or spread of a tumor or other diseased or damaged cell or tissue. For example, agonist ligand binding can stimulate the expression of a biological response modifier such as a phosphatase that inhibits cell growth or accumulation of a factor useful for the development of a cyst, such as by way of example and without limitation, phosphorylated 4EBP1. Agonists, as defined herein, without limitation, include antibodies and immunoglobulin variants, peptides, peptidomimetics, non-peptide small molecules, antisense molecules, and oligonucleotide decoys.

The term “effective amount” or “therapeutically effective amount” refers to that amount of an inhibitor, antagonist, or biological agent that is sufficient to effect the intended applications, including without limitation, clinical results as reducing the mass and/or volume of a cyst, (e.g., in the kidney in the context of PKD), inhibiting of cyst formation, restoring organ function (e.g., in the kidney in the context of PKD), decreasing symptoms resulting from the disease, increasing the quality of life of those suffering from the disease, decreasing the dose of other medications required to treat the disease, enhancing the effect of another medication, delaying the progression of the disease, and/or prolonging survival of individuals. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular antagonist chosen, the dosing regimen to be followed, whether is administered in combination with other compounds, timing of administration, the tissue to be imaged, and the physical delivery system in which it is carried.

The term “inhibit,” as used herein, refers to the ability of a compound or any agent to reduce or impede a described function, level, activity, synthesis, release, binding, etc., based on the context in which the term “inhibit” is used. The term “inhibit” is used interchangeably with “reduce,” “block,” “slow,” and “decrease.”

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. For purposes of this invention, beneficial or desired clinical results include, but are not limited to, one or more of the following: reducing the mass and/or volume of a cyst (e.g., in the kidney in the context of PKD), inhibition of cyst formation, restoring organ function (e.g., in the kidney in the context of PKD), decreasing symptoms resulting from the disease, increasing the quality of life of those suffering from the disease, decreasing the dose of other medications required to treat the disease, enhancing the effect of another medication, delaying the progression of the disease, and/or prolonging survival of individuals. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” as referred to a biologically active agent refers to the agent's ability to preferentially reduce the target signaling activity as compared to off-target signaling activity, via direct or interact interaction with the target.

“mTORC1 and/or mTORC2 activity” as applied to a biologically active agent refers to the agent's ability to modulate signal transduction mediated by mTORC1 and/or mTORC2. For example, modulation of mTORC1 and/or mTORC2 activity is evidenced by alteration in signaling output from the PI3K/Akt/mTOR pathway.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “susceptibility” or “susceptible” as used herein, refers to a subject determined to be at risk for having a disease condition. Such a determination may be based on an analysis including, but not limited to, (i) familial disease history, (ii) a genotypic characteristic of the subject, and/or (iii) a phenotypic characteristic of the subject.

The term “normalized kidney mass” as used herein, refers to combined kidney weight divided by total body weight of a mammal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC₅₀). EC₅₀ refers to the plasma concentration required for obtaining 50% of a maximum effect in vivo.

Unless otherwise stated, the connections of compound name moieties are at the rightmost recited moiety. That is, the substituent name starts with a terminal moiety, continues with any linking moieties, and ends with the linking moiety. For example, heteroarylthio C₁₋₄ alkyl has a heteroaryl group connected through a thio sulfur to a C₁₋₄ alkyl radical that connects to the chemical species bearing the substituent. This condition does not apply where a formula such as, for example “-L-C₁₋₁₀ alkyl-C₃₋₈cycloalkyl” is represented. In such case, the terminal group is a C₃₋₈ cycloalkyl group attached to a linking C₁₋₁₀ alkyl moiety which is attached to an element L, which is itself connected to the chemical species bearing the substituent.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

As used herein, for example, “C₁₋₄alkyl” is used to mean an alkyl having 1-4 carbons—that is, 1, 2, 3, or 4 carbons in a straight or branched configuration. In all embodiments of this invention, the term “alkyl” includes both branched and straight chain alkyl groups, or cyclic hydrocarbon groups, or a combination thereof. Alkyl groups are fully saturated, unsubstituted or substituted, and can include di- and multivalent radicals, having the number of carbon atoms designated (i.e. C₁-C₁₀ means one to ten carbons and C₂-C₁₀ means two to ten carbons). Typical alkyl groups are methyl, ethyl, n-propyl, isopropyl, n-butyl, sec-butyl, isobutyl, tert-butyl, n-pentyl, isopentyl, n-hexyl, n-heptyl, isooctyl, nonyl, decyl, undecyl, dodecyl, tetradecyl, hexadecyl, octadecyl, eicosyl, and the like.

The term “halo” or “halogen” refers to fluoro, chloro, bromo, or iodo.

The term “haloalkyl” refers to an alkyl group substituted with one or more halo groups, for example chloromethyl, 2-bromoethyl, 3-iodopropyl, trifluoromethyl, perfluoropropyl, 8-chlorononyl, and the like.

The term “acyl” refers to the structure —C(═O)—R, in which R is a general substituent variable such as, for example R¹ described above. Examples include, but are not limited to, alkylketo, (bi)(cyclo)alkylketo, (cyclo)alkenylketo, alkynylketo, arylketo, heteroarylketo, heterocyclylketo, heterobicycloalkylketo, spiroalkylketo. An acyl moiety is unsubstituted or is substituted on R.

Unless otherwise specified, the term “cycloalkyl” refers to a 3-8 carbon cyclic aliphatic ring structure that is unsubstituted or substituted with, for example, alkyl, hydroxy, oxo, or halo, such as cyclopropyl, methylcyclopropyl, cyclobutyl, cyclopentyl, 2-hydroxycyclopentyl, cyclohexyl, 4-chlorocyclohexyl, cycloheptyl, cyclooctyl, and the like.

The term “C₁₋₁₀alkyl-C₃₋₈cycloalkyl” is used to describe an alkyl group, branched or straight chain and containing 1 to 10 carbon atoms, attached to a linking cycloalkyl group which contains 3 to 8 carbons, such as for example, 2-methyl cyclopropyl, and the like. Either portion of the moiety is unsubstituted or substituted.

The term “bicycloalkyl” refers to a structure consisting of two cycloalkyl moieties, unsubstituted or substituted, that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[3.2.1]heptyl (“norbornyl”), bicyclo[2.2.2]octyl, and the like.

As used herein, the term “heteroatom” or “ring heteroatom” is meant to include oxygen (O), nitrogen (N), sulfur (S), phosphorus (P), and silicon (Si).

The term “heteroalkyl,” by itself or in combination with another term, means, unless otherwise stated, a straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. The alkyl portion of the moiety is unsubstituted or substituted. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R′, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “heteroalkylaryl” refers to a heteroalkyl group as defined above which is attached to an aryl group, and may be attached at a terminal point or through a branched portion of the heteroalkyl, for example, an benzyloxymethyl moiety. Either portion of the moiety is unsubstituted or substituted.

The term “heteroalkylheteroaryl” refers likewise to a heteroalkyl group which is attached to a hetaryl moiety, for example, an ethoxymethylpyridyl group. Either portion of the moiety is unsubstituted or substituted.

The term “heteroalkyl-heterocyclyl” refers to a heteroalkyl group as defined above, which is attached to a heterocyclic group, for example, 4(3-aminopropyl)-N-piperazinyl. Either portion of the moiety is unsubstituted or substituted.

The term “heteroalkyl-C3-8cycloalkyl” refers to a heteroalkyl group as defined above, which is attached to a cyclic alkyl containing 3 to 8 carbons, for example, 1-aminobutyl-4-cyclohexyl. Either portion of the moiety is unsubstituted or substituted.

The term “heterobicycloalkyl” refers to a bicycloalkyl structure, which is unsubstituted or substituted, in which at least one carbon atom is replaced with a heteroatom independently selected from oxygen, nitrogen, and sulfur.

The term “heterospiroalkyl” refers to a spiroalkyl structure, which is unsubstituted or substituted, in which at least one carbon atom is replaced with a heteroatom independently selected from oxygen, nitrogen, and sulfur. “Alkenyl” refers to a straight or branched hydrocarbon chain radical group containing at least one double bond, and having from two to ten carbon atoms (ie. C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to five carbon atoms (e.g., C₂-C₅ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. The alkenyl is unsubstituted or substituted. The term “C₂₋₁₀ alkenyl-C₃₋₈cycloalkyl” refers to a group containing an alkenyl group, containing 2 to 10 carbons and branched or straight chain, which is attached to a linking cycloalkyl group containing 3 to 8 carbons, such as, for example 3-prop-3-enyl-cyclopent-1yl, and the like. Either portion of the moiety is unsubstituted or substituted.

The term “C₂₋₁₀ alkenyl-heteroalkyl” refers to a group having an alkenyl moiety, containing 2 to 10 carbon atoms and is branched or straight chain, which is attached to a linking heteroalkyl group, such as, for example, allyloxy, and the like. Either portion of the moiety is unsubstituted or substituted.

The term “C₂₋₁₀ alkynyl-heteroalkyl” refers to a group having an alkynyl moiety, which is unsubstituted or substituted, containing 2 to 10 carbon atoms and is branched or straight chain, which is attached to a linking heteroalkyl group, such as, for example, 4-but-1-ynoxy, and the like. Either portion of the moiety is unsubstituted or substituted.

The term “haloalkenyl” refers to an alkenyl group substituted with one or more halo groups.

Unless otherwise specified, the term “cycloalkenyl” refers to a cyclic aliphatic 3 to 8 membered ring structure, optionally substituted having 1 or more ethylenic bonds such as methylcyclopropenyl, trifluoromethylcyclopropenyl, cyclopentenyl, cyclohexenyl, 1,4-cyclohexadienyl, and the like. In some embodiments, a cycloalkenyl may be substituted with one or more alkyl, hydroxyl, or halo.

“Alkynyl” refers to a straight or branched hydrocarbon chain radical group containing at least one triple bond, having from two to ten carbon atoms (ie. C₂-C₁₀ alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to five carbon atoms (e.g., C₂-C₅ alkynyl). The alkynyl is attached to the rest of the molecule by a single bond, for example, ethynyl, propynyl, butynyl, pentynyl, hexynyl, and the like. The alkynyl is unsubstituted or substituted.

The term C₂₋₁₀ alkynyl-C₃₋₈ cycloalkyl refers to a group containing an alkynyl group, containing 2 to 10 carbons and branched or straight chain, which is attached to a linking cycloalkyl group containing 3 to 8 carbons, such as, for example 3-prop-3-ynyl-cyclopent-1yl, and the like. Either portion of the moiety is unsubstituted or substituted.

The term “haloalkynyl” refers to an alkynyl group substituted with one or more independent halo groups.

“Amino” or “amine” refers to a —NR′R″ moiety, where each R and R″ are independently hydrogen, alkyl, alkenyl, alkynyl, heteroalkyl, alkenyl-heteroalkyl, alkynyl-heteroalkyl, fluoroalkyl, haloalkyl, cycloalkyl, cycloalkylalkyl, aryl, aralkyl (arylalkyl), heterocyclyl, heterocyclylalkyl, heteroaryl or heteroarylalkyl, unless stated otherwise specifically in the specification. When both R′ and R″ of a —NR′R″ moiety are not hydrogen, R′ and R″ can be combined with the nitrogen atom to form a 4-, 5-, 6-, or 7-membered ring. For example, —NR′R″ is meant to include, but not be limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR′, —SR′, —OC(O)—R′, —N(R′)₂, —C(O)R′, —C(O)OR′, —OC(O)N(R′)₂, —C(O)N(R′)₂, —N(R′)C(O)OR′, —N(R′)C(O)R′, —N(R′)C(O)N(R′)₂, N(R′)C(NR′)N(R′)₂, —N(R′)S(O)ₜR′ (where t is 1 or 2), —S(O)ₜOR′ (where t is 1 or 2), —S(O)ₜN(R′)₂ (where t is 1 or 2), or PO₃(R′)₂, where each R′ is independently hydrogen, alkyl, alkenyl, alkynyl, fluoroalkyl, haloalkyl, cycloalkyl, cycloalkylalkyl, heteroalkyl, alkenyl-heteroalkyl, alkynyl-heteroalkyl, carbocyclyl, carbocyclylalkyl, aryl, aralkyl, heterocyclyl, heterocyclylalkyl, heteroaryl or heteroarylalkyl, wherein each may be substituted or unsubstituted.

“Amide” or “amido” refers to a chemical moiety with formula —C(O)N(R′)₂ or —NHC(O)R′, where R′ is independently selected from the group consisting of hydrogen, alkyl, cycloalkyl, aryl, heteroaryl (bonded through a ring carbon), heteroalicyclic (bonded through a ring carbon), alkenyl, alkynyl, heteroalkyl, alkenyl-heteroalkyl, alkynyl-heteroalkyl, fluoroalkyl, haloalkyl, cycloalkyl, cycloalkylalkyl, aryl, aralkyl (arylalkyl), heterocyclyl, heterocyclylalkyl, heteroaryl or heteroarylalkyl, wherein each may be substituted or unsubstituted, unless stated otherwise specifically in the specification. In some embodiments it is a C₁-C₄ amido or amide radical, which includes the amide carbonyl in the total number of carbons in the radical. The (R′)₂ of —N(R′)₂ of the amide may optionally be taken together with the nitrogen to which it is attached to form a 4-, 5-, 6-, or 7-membered ring. Unless stated otherwise specifically in the specification, an amido group is optionally substituted independently by one or more of the substituents as described herein for alkyl, cycloalkyl, aryl, heteroaryl, or heterocyclyl. An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety.

“Aromatic” or “aryl” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Examples of aryl include, but are not limited to, phenyl, 4-chlorophenyl, 4-fluorophenyl, 4-bromophenyl, 3-nitrophenyl, 2-methoxyphenyl, 2-methylphenyl, 3-methylphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, and 2-iodo-4-methylphenyl. An aryl moiety is unsubstituted or substituted.

“Heteroaryl” or, alternatively, “heteroaromatic”, “heteroaryl”, “heteroar” or “hetar” refers to a 5- to 18-membered aromatic radical (e.g., C₅-C₁₈ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range; e.g., “5 to 18 ring atoms” means that the heteroaryl group may consist of 5 ring atoms, 6 ring atoms, etc., up to and including 18 ring atoms. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). A heteroaryl moiety is unsubstituted or substituted.

The terms “aryl-alkyl”, “arylalkyl” and “aralkyl” are used to describe a group wherein the alkyl chain can be branched or straight chain forming a linking portion with the terminal aryl, as defined above, of the aryl-alkyl moiety. Examples of aryl-alkyl groups include, but are not limited to, optionally substituted benzyl, phenethyl, phenpropyl and phenbutyl such as 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitrophenyl)ethyl, 2-(2,4-dichlorophenyl)ethyl, 2-(3,5-dimethoxyphenyl)ethyl, 3-phenylpropyl, 3-(3-chlorophenyl)propyl, 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxyphenyl)butyl, and 10-phenyldecyl. Either portion of the moiety is unsubstituted or substituted.

The term “C₁₋₁₀alkylaryl” as used herein refers to a terminal alkyl group, as defined above, containing 1 to 10 carbon atoms, branched or unbranched, attached to a linking aryl group, wherein the aryl group replaces one hydrogen on the alkyl group, for example, 3-phenylpropyl. Either portion of the moiety is unsubstituted or substituted.

The term C₂₋₁₀ alkyl monocycloaryl” refers to a group containing a terminal alkyl group, branched or straight chain and containing 2 to 10 atoms attached to a linking aryl group which has only one ring, such as for example, 2-phenyl ethyl. Either portion of the moiety is unsubstituted or substituted.

The term “C₁₋₁₀ alkyl bicycloaryl” refers to a group containing a terminal alkyl group, branched or straight chain and containing 2 to 10 atoms attached to a linking aryl group which is bicyclic, such as for example, 2-(1-naphthyl)-ethyl. Either portion of the moiety is unsubstituted or substituted.

The terms “aryl-cycloalkyl” and “arylcycloalkyl” are used to describe a group wherein the terminal aryl group is attached to a cycloalkyl group, for example phenylcyclopentyl and the like. Either portion of the moiety is unsubstituted or substituted.

The terms “heteroaryl-C₃₋₈cycloalkyl” and “heteroaryl-C₃₋₈cycloalkyl” are used to describe a group wherein the terminal heteroaryl group is attached to a cycloalkyl group, which contains 3 to 8 carbons, for example pyrid-2-yl-cyclopentyl and the like. Either portion of the moiety is unsubstituted or substituted.

The term “heteroaryl-heteroalkyl” refers to a group wherein the terminal heteroaryl group is attached to a linking heteroalkyl group, such as for example, pyrid-2-yl methylenoxy, and the like. Either portion of the moiety is unsubstituted or substituted.

The terms “aryl-alkenyl”, “arylalkenyl” and “aralkenyl” are used to describe a group wherein the alkenyl chain can be branched or straight chain forming a linking portion of the aralkenyl moiety with the terminal aryl portion, as defined above, for example styryl (2-phenylvinyl), phenpropenyl, and the like. Either portion of the moiety is unsubstituted or substituted.

The term “aryl-C₂₋₁₀alkenyl” means an arylalkenyl as described above wherein the alkenyl moiety contains 2 to 10 carbon atoms such as for example, styryl (2-phenylvinyl), and the like. Either portion of the moiety is unsubstituted or substituted.

The term “C₂₋₁₀alkenyl-aryl” is used to describe a group wherein the terminal alkenyl group, which contains 2 to 10 carbon atoms and can be branched or straight chain, is attached to the aryl moiety which forms the linking portion of the alkenyl-aryl moiety, such as for example, 3-propenyl-naphth-1-yl, and the like. Either portion of the moiety is unsubstituted or substituted.

The terms “aryl-alkynyl”, “arylalkynyl” and “aralkynyl” are used to describe a group wherein the alkynyl chain can be branched or straight chain forming a linking portion of the aryl-alkynyl moiety with the terminal aryl portion, as defined above, for example 3-phenyl-1-propynyl, and the like. Either portion of the moiety is unsubstituted or substituted.

The term “aryl-C₂₋₁₀alkynyl” means an arylalkynyl as described above wherein the alkynyl moiety contains two to ten carbons, such as, for example 3-phenyl-1-propynyl, and the like. Either portion of the moiety is unsubstituted or substituted.

The term “C₂₋₁₀alkynyl-aryl” means a group containing an alkynyl moiety attached to an aryl linking group, both as defined above, wherein the alkynyl moiety contains two to ten carbons, such as, for example 3-propynyl-naphth-1-yl. Either portion of the moiety is unsubstituted or substituted.

The terms “aryl-oxy”, “aryloxy” and “aroxy” are used to describe a terminal aryl group attached to a linking oxygen atom. Typical aryl-oxy groups include phenoxy, 3,4-dichlorophenoxy, and the like. Either portion of the moiety is unsubstituted or substituted.

The terms “aryl-oxyalkyl”, “aryloxyalkyl” and “aroxyalkyl” are used to describe a group wherein an alkyl group is substituted with a terminal aryl-oxy group, for example pentafluorophenoxymethyl and the like. Either portion of the moiety is unsubstituted or substituted.

The term “C₁₋₁₀alkoxy-C₁₋₁₀alkyl” refers to a group wherein an alkoxy group, containing 1 to 10 carbon atoms and an oxygen atom within the branching or straight chain, is attached to a linking alkyl group, branched or straight chain which contains 1 to 10 carbon atoms, such as, for example methoxypropyl, and the like. Either portion of the moiety is unsubstituted or substituted.

The term “C₁₋₁₀alkoxy-C₂₋₁₀alkenyl” refers to a group wherein an alkoxy group, containing 1 to 10 carbon atoms and an oxygen atom within the branching or straight chain, is attached to a linking alkenyl group, branched or straight chain which contains 1 to 10 carbon atoms, such as, for example 3-methoxybut-2-en-1-yl, and the like. Either portion of the moiety is unsubstituted or substituted.

The term “C₁₋₁₀alkoxy-C₂₋₁₀alkynyl” refers to a group wherein an alkoxy group, containing 1 to 10 carbon atoms and an oxygen atom within the branching or straight chain, is attached to a linking alkynyl group, branched or straight chain which contains 1 to 10 carbon atoms, such as, for example 3-methoxybut-2-in-1-yl, and the like. Either portion of the moiety is unsubstituted or substituted.

The terms “hetaryl-oxy”, “heteroaryl-oxy”, “hetaryloxy”, “heteroaryloxy”, “hetaroxy” and “heteroaroxy” are used to describe a terminal hetaryl group, which is unsubstituted or substituted, attached to a linking oxygen atom. Typical hetaryl-oxy groups include 4,6-dimethoxypyrimidin-2-yloxy and the like.

The terms “hetarylalkyl”, “heteroarylalkyl”, “hetaryl-alkyl”, “heteroaryl-alkyl”, “hetaralkyl” and “heteroaralkyl” are used to describe a group wherein the alkyl chain can be branched or straight chain forming a linking portion of the heteroaralkyl moiety with the terminal heteroaryl portion, as defined above, for example 3-furylmethyl, thienyl, furfuryl, and the like. Either portion of the moiety is unsubstituted or substituted. The term “heteroaryl-C₁₋₁₀alkyl” is used to describe a heteroaryl alkyl group as described above where the alkyl group contains 1 to 10 carbon atoms. Either portion of the moiety is unsubstituted or substituted.

The term “C₁₋₁₀alkyl-heteroaryl” is used to describe a alkyl attached to a heteroaryl group as described above where the alkyl group contains 1 to 10 carbon atoms. Either portion of the moiety is unsubstituted or substituted.

The terms “heteroarylalkenyl”, “heteroarylalkenyl”, “heteroaryl-alkenyl”, “heteroaryl-alkenyl”, “hetaralkenyl” and “heteroaralkenyl” are used to describe a heteroarylalkenyl group wherein the alkenyl chain can be branched or straight chain forming a linking portion of the heteroaralkenyl moiety with the terminal heteroaryl portion, as defined above, for example 3-(4-pyridyl)-1-propenyl. Either portion of the moiety is unsubstituted or substituted.

The term “heteroaryl-C₂₋₁₀alkenyl” group is used to describe a group as described above wherein the alkenyl group contains 2 to 10 carbon atoms. Either portion of the moiety is unsubstituted or substituted.

The term “C₂₋₁₀alkenyl-heteroaryl” is used to describe a group containing an alkenyl group, which is branched or straight chain and contains 2 to 10 carbon atoms, and is attached to a linking heteroaryl group, such as, for example 2-styryl-4-pyridyl, and the like. Either portion of the moiety is unsubstituted or substituted.

The terms “heteroarylalkynyl”, “heteroarylalkynyl”, “heteroaryl-alkynyl”, “heteroaryl-alkynyl”, “hetaralkynyl” and “heteroaralkynyl” are used to describe a group wherein the alkynyl chain can be branched or straight chain forming a linking portion of the heteroaralkynyl moiety with the heteroaryl portion, as defined above, for example 4-(2-thienyl)-1-butynyl, and the like. Either portion of the moiety is unsubstituted or substituted.

The term “heteroaryl-C₂₋₁₀alkynyl” is used to describe a heteroarylalkynyl group as described above wherein the alkynyl group contains 2 to 10 carbon atoms. Either portion of the moiety is unsubstituted or substituted.

The term “C₂₋₁₀alkynyl-heteroaryl” is used to describe a group containing an alkynyl group which contains 2 to 10 carbon atoms and is branched or straight chain, which is attached to a linking heteroaryl group such as, for example, 4(but-1-ynyl) thien-2-yl, and the like. Either portion of the moiety is unsubstituted or substituted.

The term “heterocyclyl”, “hetcyclyl”, or “heterocycloalkyl” refers to a substituted or unsubstituted 3-, 4-, 5-, or 6-membered saturated or partially unsaturated ring containing one, two, or three heteroatoms, preferably one or two heteroatoms independently selected from oxygen, nitrogen and sulfur; or to a bicyclic ring system containing up to 10 atoms including at least one heteroatom independently selected from oxygen, nitrogen, and sulfur. Examples of heterocyclyls include, but are not limited to, tetrahydrofuranyl, tetrahydrofuryl, pyrrolidinyl, piperidinyl, 4-pyranyl, tetrahydropyranyl, thiolanyl, morpholinyl, piperazinyl, dioxolanyl, dioxanyl, indolinyl, and 5-methyl-6-chromanyl.

The terms “heterocyclylalkyl”, “heterocyclyl-alkyl”, “hetcyclylalkyl”, and “hetcyclyl-alkyl” are used to describe a group wherein the alkyl chain can be branched or straight chain forming a linking portion of the heterocyclylalkyl moiety with the terminal heterocyclyl portion, as defined above, for example 3-piperidinylmethyl and the like. The term “heterocycloalkylene” refers to the divalent derivative of heterocycloalkyl.

The term “C₁₋₁₀alkyl-heterocycyl” refers to a group as defined above where the alkyl moiety contains 1 to 10 carbon atoms attached to a linking heterocyclyl. Either portion of the moiety is unsubstituted or substituted.

The term “heterocycyl-C₁₋₁₀alkyl” refers to a group containing a terminal heterocyclic group attached to a linking alkyl group which contains 1 to 10 carbons and is branched or straight chain, such as, for example, 4-morpholinyl ethyl, and the like. Either portion of the moiety is unsubstituted or substituted.

The terms “heterocyclylalkenyl”, “heterocyclyl-alkenyl”, “hetcyclylalkenyl” and “hetcyclyl-alkenyl” are used to describe a group wherein the alkenyl chain can be branched or straight chain forming a linking portion of the heterocyclylalkenyl moiety with the terminal heterocyclyl portion, as defined above, for example 2-morpholinyl-1-propenyl and the like. The term “heterocycloalkenylene” refers to the divalent derivative of heterocyclylalkenyl. Either portion of the moiety is unsubstituted or substituted.

The term “heterocycyl-C₂₋₁₀ alkenyl” refers to a group as defined above where the alkenyl group contains 2 to 10 carbon atoms and is branched or straight chain, such as, for example, 4-(N-piperazinyl)-but-2-en-1-yl, and the like. Either portion of the moiety is unsubstituted or substituted.

The terms “heterocyclylalkynyl”, “heterocyclyl-alkynyl”, “hetcyclylalkynyl” and “hetcyclyl-alkynyl” are used to describe a group wherein the alkynyl chain can be branched or straight chain forming a linking portion of the heterocyclylalkynyl moiety with the terminal heterocyclyl portion, as defined above, for example 2-pyrrolidinyl-1-butynyl and the like. Either portion of the moiety is unsubstituted or substituted.

The term “heterocycyl-C₂₋₁₀ alkynyl” refers to a group as defined above where the alkynyl group contains 2 to 10 carbon atoms and is branched or straight chain, such as, for example, 4-(N-piperazinyl)-but-2-yn-1-yl, and the like.

The term “aryl-heterocyclyl” refers to a group containing a terminal aryl group attached to a linking heterocyclic group, such as for example, N4-(4-phenyl)-piperazinyl, and the like. Either portion of the moiety is unsubstituted or substituted.

The term “heteroaryl-heterocyclyl” refers to a group containing a terminal heteroaryl group attached to a linking heterocyclic group, such as for example, N4-(4-pyridyl)-piperazinyl, and the like. Either portion of the moiety is unsubstituted or substituted.

The terms “cycloalkylalkyl” and “cycloalkyl-alkyl” refer to a terminal cycloalkyl group as defined above attached to an alkyl group, for example cyclopropylmethyl, cyclohexylethyl, and the like. Either portion of the moiety is unsubstituted or substituted.

The terms “cycloalkylalkenyl” and “cycloalkyl-alkenyl” refer to a terminal cycloalkyl group as defined above attached to an alkenyl group, for example cyclohexylvinyl, cycloheptylallyl, and the like. Either portion of the moiety is unsubstituted or substituted.

The terms “cycloalkylalkynyl” and “cycloalkyl-alkynyl” refer to a terminal cycloalkyl group as defined above attached to an alkynyl group, for example cyclopropylpropargyl, 4-cyclopentyl-2-butynyl, and the like. Either portion of the moiety is unsubstituted or substituted.

The term “alkoxy” includes both branched and straight chain terminal alkyl groups attached to a linking oxygen atom. Typical alkoxy groups include methoxy, ethoxy, n-propoxy, isopropoxy, tert-butoxy and the like. An alkoxy moiety is unsubstituted or substituted.

The term “haloalkoxy” refers to an alkoxy group substituted with one or more halo groups, for example chloromethoxy, trifluoromethoxy, difluoromethoxy, perfluoroisobutoxy, and the like.

The term “alkoxyalkoxyalkyl” refers to an alkyl group substituted with an alkoxy moiety which is in turn is substituted with a second alkoxy moiety, for example methoxymethoxymethyl, isopropoxymethoxyethyl, and the like. This moiety is substituted with further substituents or not substituted with other substituents.

The term “alkylthio” includes both branched and straight chain alkyl groups attached to a linking sulfur atom, for example methylthio and the like.

The term “alkoxyalkyl” refers to an alkyl group substituted with an alkoxy group, for example isopropoxymethyl and the like. Either portion of the moiety is unsubstituted or substituted.

The term “alkoxyalkenyl” refers to an alkenyl group substituted with an alkoxy group, for example 3-methoxyallyl and the like. Either portion of the moiety is unsubstituted or substituted.

The term “alkoxyalkynyl” refers to an alkynyl group substituted with an alkoxy group, for example 3-methoxypropargyl and the like. Either portion of the moiety is unsubstituted or substituted.

The term “C₁₋₁₀alkylC₃₋₈cycloalkyl” refers to an alkyl group having 1 to 10 carbons, attached to a linking three to eight membered cycloalkyl group. Either portion of the moiety is unsubstituted or substituted.

The term “C₂₋₁₀alkenylC₃₋₈cycloalkyl” refers to an alkenyl group as defined above attached to a linking three to eight membered cycloalkyl group, for example, 4-(cyclopropyl)-2-butenyl and the like. Either portion of the moiety is unsubstituted or substituted.

The term “C₂₋₁₀alkynylC₃₋₈cycloalkyl” refers to an alkynyl group as defined attached to a linking three to eight membered cycloalkyl group, for example, 4-(cyclopropyl)-2-butynyl and the like. Either portion of the moiety is unsubstituted or substituted.

The term “heterocyclyl-C₁₋₁₀alkyl” refers to a heterocyclic group as defined above attached to a linking alkyl group as defined above having 1 to 10 carbons, for example, 4-(N-methyl)-piperazinyl, and the like. Either portion of the moiety is unsubstituted or substituted.

The term “heterocyclyl-C₂₋₁₀alkenyl” refers to a heterocyclic group as defined above, attached to a linking alkenyl group as defined above, having 2 to 10 carbons, for example, 4-(N-allyl)piperazinyl, and the like. Moieties wherein the heterocyclic group is substituted on a carbon atom with an alkenyl group are also included. Either portion of the moiety is unsubstituted or substituted.

The term “heterocyclyl-C₂₋₁₀alkynyl” refers to a heterocyclic group as defined above, attached to a linking alkynyl group as defined above, having 2 to 10 carbons, for example, 4-(N-propargyl)piperazinyl, and the like. Moieties wherein the heterocyclic group is substituted on a carbon atom with an alkynyl group are also included. Either portion of the moiety is unsubstituted or substituted.

The term “oxo” refers to an oxygen that is double bonded to a carbon atom. One in the art understands that an “oxo” requires a second bond from the atom to which the oxo is attached. Accordingly, it is understood that oxo cannot be substituted onto an aryl or heteroaryl ring, unless it forms part of the aromatic system as a tautomer.

“Sulfonamidyl” or “sulfonamido” refers to a —S(═O)₂—NR′R′ radical, where each R′ is selected independently from the group consisting of hydrogen, alkyl, cycloalkyl, aryl, heteroaryl (bonded through a ring carbon), heteroalicyclic (bonded through a ring carbon), alkenyl, alkynyl, heteroalkyl, alkenyl-heteroalkyl, alkynyl-heteroalkyl, fluoroalkyl, haloalkyl, cycloalkyl, cycloalkylalkyl, aryl, aralkyl (arylalkyl), heterocyclyl, heterocyclylalkyl, heteroaryl or heteroarylalkyl, unless stated otherwise specifically in the specification. The R′ groups in —NR′R′ of the —S(═O)₂—NR′R′ radical may be taken together with the nitrogen to which it is attached to form a 4-, 5-, 6-, or 7-membered ring. A sulfonamido group is optionally substituted by one or more of the substituents described for alkyl, cycloalkyl, aryl, heteroaryl respectively.

Compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The present invention includes all manner of rotamers and conformationally restricted states of a compound of the invention.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: alkyl, heteroalkyl, alkenyl, alkynyl, cycloalkyl, heterocycloalkyl, aryl, arylalkyl, heteroaryl, heteroarylalkyl, —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted heteroalkenyl, substituted or unsubstituted heteroalkynyl, substituted or unsubstituted cycloalkyl, substituted or unsubstituted heterocycloalkyl, substituted or unsubstituted cycloalkenyl, substituted or unsubstituted cycloalkynyl, substituted or unsubstituted aryl (e.g., aryl substituted with 1-3 halogens), substituted or unsubstituted heteroaryl, substituted or unsubstituted alkyl, substituted or unsubstituted alkenyl, substituted or unsubstituted alkynyl, alkoxy or thioalkoxy groups, or arylalkyl groups. When a compound of the invention includes more than one R group, for example, each of the R groups is independently selected as are each R′, R″, R′″ and R″″ groups when more than one of these groups is present.

When R′ and R″ or R″ and R′″ are attached to the same nitrogen atom, they can be combined with the nitrogen atom to form a 4-, 5-, 6-, or 7-membered ring. For example, —NR′R″ is meant to include, but not be limited to, 1-pyrrolidinyl, 4 piperazinyl, and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, alkyl, heteroalkyl, alkenyl, alkynyl, cycloalkyl, heterocycloalkyl, aryl, arylalkyl, heteroaryl, heteroarylalkyl, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ are preferably independently selected from hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted cycloalkyl, substituted or unsubstituted cycloalkenyl, substituted or unsubstituted cycloalkynyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Two of the substituents on adjacent atoms of aryl or heteroaryl ring may optionally form a ring of the formula-T-C(O)—(CRR′)q-U—, wherein T and U are independently —NR—, -0-, —CRR′— or a single bond, and q is an integer of from 0 to 3. Alternatively, two of the substituents on adjacent atoms of aryl or heteroaryl ring may optionally be replaced with a substituent of the formula -A-(CH2)r-B—, wherein A and B are independently —CRR′—, -0-, —NR—, —S—, —S(O)—, —S(O)z-, —S(0)2NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CRR′)s—X′(C″R′″)d-, where sand dare independently integers of from 0 to 3, and X′ is -0-, —NR′—, —S—, —S(O)—, S(O)z-, or —S(0)2NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

### II. Methods

A. Compounds

Provided herein, inter alia, are methods of use of a distinct class of biologically active agents that exhibit selective inhibition of certain protein kinases, and the uses of these agents for treatment of diseases mediated by such protein kinases. In some embodiments, a method of treatment is provided, including administration of a compound as described herein (including embodiments) to a patient in need. In one embodiment, the present invention provides for a method for treating an autosomal polycystic kidney disorder comprising contacting a cell with a compound (e.g. an inhibitor that selectively inhibits mTORC1 and mTORC2 activity), wherein the compound (e.g. mTOR inhibitor) is a compound of Formula I. In some embodiments, a method is provided for treating an autosomal polycystic kidney disorder including contacting a cell with a compound that selectively inhibits mTORC1 or mTORC2 activity, wherein the compound (e.g. mTOR inhibitor) is a compound of Formula I. In one embodiment, the compound (e.g. mTOR inhibitor) inhibits mTOR relative to one or more type I phosphatidylinositol 3-kinases (PI3-kinase), wherein the one or more type I PI3-kinase is selected from the group consisting of PI3-kinase α, PI3-kinase β, PI3-kinase γ, and PI3-kinase δ. In one embodiment, the compound (e.g. mTOR inhibitor) inhibits mTOR activity more than the compound (e.g. mTOR inhibitor) inhibits the activity of one or more type I phosphatidylinositol 3-kinases (PI3-kinase), wherein the one or more type I PI3-kinase is selected from the group consisting of PI3-kinase α, PI3-kinase β, PI3-kinase γ, and PI3-kinase δ. In some embodiments, the level of activity inhibition is measured as a percentage decrease in activity at a specific amount of compound (e.g. mTOR inhibitor) administered. In some embodiments, the level of activity inhibition is measured as an IC50 or an EC50, wherein a greater level of inhibition is shown by a lower concentration of compound (e.g. mTOR inhibitor) needed to reduce the activity by 50% in an assay for measuring IC50 or EC50. In some embodiments, the level of activity inhibition is measured as a Kd (dissociation constant), wherein a lower Kd value indicates greater inhibition (e.g. nanomolar is better than micromolar). It is generally recognized that there are four types of PI3K: IA, IB, II and III. Type IA enzymes act downstream of tyrosine kinases to generate phosphatidylinositol-3,4,5-trisphosphate (PIP3), a crucial second messenger that promotes proliferation and transformation. Class IA enzymes typically exist as dimers of a 110 kDa catalytic subunit (p110α, p110β or p¹¹⁰δ) and a regulatory subunit of varying size. The single class IB PI3K enzyme, p110γ, is activated downstream of G protein-coupled receptors.

Any agents (e.g. compound of Formula (I)) that selectively and negatively regulate mTORC1 and/or mTOR2C expression or activity can be used as selective mTOR inhibitors in the methods of the invention. The relative efficacies of agents as inhibitors of mTORC1 or mTORC2 can be established by determining the concentrations at which each agent inhibits the activity to a predefined extent.

In one aspect, a determination is the concentration that inhibits 50% of the activity in a cell-based assay or in an in vitro kinase assay. IC₅₀ determinations can be accomplished using any conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the “IC₅₀” value. Analogously, other inhibitory concentrations can be defined through appropriate determinations of activity. For example, in some settings it can be desirable to establish a 90% inhibitory concentration, i.e., IC₉₀, etc.

In some embodiments, an in vitro kinase assay includes the use of labeled ATP as phosphodonor, and following the kinase reaction the substrate peptide is captured on an appropriate filter. Unreacted labeled ATP and metabolites are resolved from the radioactive peptide substrate by various techniques, involving trichloroacetic acid precipitation and extensive washing. Addition of several positively charged residues allows capture on phosphocellulose paper followed by washing. Radioactivity incorporated into the substrate peptide is detected by scintillation counting. This assay is relatively simple, reasonably sensitive, and the peptide substrate can be adjusted both in terms of sequence and concentration to meet the assay requirements.

Other exemplary kinase assays are detailed in U.S. Pat. No. 5,759,787 and U.S. application Ser. No. 12/728,926, both of which are incorporated herein by reference in their entirety. Exemplary compounds (e.g. mTOR inhibitor) for use in the invention are disclosed in U.S. application Ser. No. 12/586,309, filed on Sep. 17, 2009, which is incorporated herein by reference in its entirety and for all purposes. Additional exemplary compounds (e.g. mTOR inhibitor) for use in the invention are disclosed in U.S. application Ser. No. 12/920,970, filed on Sep. 3, 2010, which is incorporated herein by reference in its entirety and for all purposes.

Alternatively, IC₅₀ determinations can be accomplished by measuring the phosphorylation level of substrate proteins of the target in a cell-based assay. For example, one substrate of mTOR is AKT, which may be phosphorylated at T308 or S473. Cells, for example, may be contacted with the inhibitor under study under conditions, such as 100 nM insulin, which would normally yield phosphorylation of mTOR substrates including but not limited to AKT at S473 and T308. Cells may then be prepared by various methods known to the art including fixation or lysis, and analyzed for the phosphorylation levels of mTOR substrates. Optionally, specificity or selectivity may be determined by examining the effect of the inhibitor under study on the phosphorylation of substrates of other kinases. Phosphorylation levels may be analyzed using any methods known to the art including but not limited to the use of antibodies specific for the phosphorylated forms of the substrates to be assayed via immunoblot or flow cytometry.

In another aspect, a selective mTOR inhibitor alternatively can be understood to refer to an agent of Formula I that exhibits a 50% inhibitory concentration (IC₅₀) with respect to mTORC1 and/or mTORC2, that is at least at least 10-fold, at least 20-fold, at least 50-fold, at least 100-fold, at least 1000-fold, at least 10,100-fold, or more, lower than the inhibitor's IC₅₀ with respect to one, two, three, or more type I PI3-kinases. In some embodiment, a selective mTOR inhibitor of Formula I alternatively can be understood to refer to an agent that exhibits a 50% inhibitory concentration (IC₅₀) with respect to mTORC1 and/or mTORC2, that is at least 10-fold, at least 20-fold, at least 50-fold, at least 100-fold, at least 1000-fold, at least 10,000-fold, or more, lower than the inhibitor's IC₅₀ with respect to all of type I PI3-kinases.

In yet another aspect, a selective mTOR inhibitor alternatively can be understood to refer to a text use compound that exhibits a 50% inhibitory concentration (IC₅₀) with respect to mTOR, that is at least 10-fold, at least 20-fold, at least 50-fold, at least 100-fold, at least 1000-fold, at least 10,000-fold, or lower, than the inhibitor's IC₅₀ with respect to one or more protein kinases selected from the group consisting of PKC □I, PKC

[symbol]

The subject biologically active agent may inhibit both mTORC1 and mTORC2 activity with an IC₅₀ value of about 100 nM or less, preferably about 50 nM, about 25 nM, about 10 nM, about 5 nM, about 1 nM, 100 pM, 50 pM, 25 pM, 10 pM, 1 pM, or less, as ascertained in a cell-based assay or an in vitro kinase assay.

Inhibition of mTORC1 and/or mTORC2 activity can be determined by a reduction in signal transduction of the PI3K/Akt/mTOR pathway. A wide variety of readouts can be utilized to establish a reduction of the output of such signaling pathway. Some non-limiting exemplary readouts include (1) a decrease in phosphorylation of Akt at residues, including but not limited to S473 and T308; (2) a decrease in activation of Akt as evidenced by a reduction of phosphorylation of Akt substrates including but not limited to FoxO1/O3a T24/32, GSK3α/β S21/9, and TSC2 T1462; (3) a decrease in phosphorylation of signaling molecules downstream of mTOR, including but not limited to ribosomal S6 S240/244, 70S6K T389, and 4EBP1 T37/46; (4) inhibition of proliferation of cells including but not limited to normal or neoplastic cells, mouse embryonic fibroblasts, and epithelial cells; (5) induction of apoptosis of cells or cell cycle arrest; (6) reduction of cell chemotaxis; and (7) an increase in binding of 4EBP1 to eIF4E. The term “eIF4E” refers to a 24-kD eukaryotic translation initiation factor involved in directing ribosomes to the cap structure of mRNAs, having human gene locus 4q21-q25.

mTOR exists in two types of complexes, mTORC1 containing the raptor subunit and mTORC2 containing rictor. As known in the art, “rictor” refers to a cell growth regulatory protein having human gene locus 5p13.1. These complexes are regulated differently and have a different spectrum of substrates. For instance, mTORC1 phosphorylates S6 kinase (S6K) and 4EBP1, promoting increased translation and ribosome biogenesis to facilitate cell growth and cell cycle progression. S6K also acts in a feedback pathway to attenuate PI3K/Akt activation. Thus, inhibition of mTORC1 (e.g. by a biologically active agent as discussed herein) results in activation of 4EBP1, resulting in inhibition of (e.g. a decrease in) RNA translation.

mTORC2 is generally insensitive to rapamycin and selective inhibitors. mTORC2 is thought to modulate growth factor signaling by phosphorylating the C-terminal hydrophobic motif of some AGC kinases such as Akt. In many cellular contexts, mTORC2 is required for phosphorylation of the S473 site of Akt. Thus, mTORC1 activity is partly controlled by Akt whereas Akt itself is partly controlled by mTORC2.

Growth factor stimulation of PI3K causes activation of Akt by phosphorylation at the two key sites, S473 and T308. It has been reported that full activation of Akt requires phosphorylation of both S473 and T308. Akt promotes cell survival and proliferation in many ways including suppressing apoptosis, promoting glucose uptake, and modifying cellular metabolism. Of the two phosphorylation sites on Akt, activation loop phosphorylation at T308, mediated by PDK1, is believed to be indispensable for kinase activity, while hydrophobic motif phosphorylation at S473 enhances Akt kinase activity.

Selective mTOR inhibition may also be determined by expression levels of the mTOR genes, its downstream signaling genes (for example by RT-PCR), or expression levels of the proteins (for example by immunocytochemistry, immunohistochemistry, Western blots) as compared to other PI3-Kinases or protein kinases.

Cell-based assays for establishing selective inhibition of mTORC1 and/or mTORC2 can take a variety of formats. This generally will depend on the biological activity and/or the signal transduction readout that is under investigation. For example, the ability of the agent to inhibit mTORC1 and/or mTORC2 to phosphorylate the downstream substrate(s) can be determined by various types of kinase assays known in the art. Representative assays include but are not limited to immunoblotting and immunoprecipitation with antibodies such as anti-phosphotyrosine, anti-phosphoserine or anti-phosphothreonine antibodies that recognize phosphorylated proteins. Alternatively, antibodies that specifically recognize a particular phosphorylated form of a kinase substrate (e.g., anti-phospho AKT S473 or anti-phospho AKT T308) can be used. In addition, kinase activity can be detected by high throughput chemiluminescent assays such as AlphaScreen™ (available from Perkin Elmer) and eTag™ assay (Chan-Hui, et al. (2003) Clinical Immunology 111: 162-174). In another aspect, single cell assays such as flow cytometry as described in the phosflow experiment can be used to measure phosphorylation of multiple downstream mTOR substrates in mixed cell populations.

One advantage of the immunoblotting and phosflow methods is that the phosphorylation of multiple kinase substrates can be measured simultaneously. This provides the advantage that efficacy and selectivity can be measured at the same time. For example, cells may be contacted with a compound (e.g. mTOR inhibitor) at various concentrations and the phosphorylation levels of substrates of both mTOR and other kinases can be measured. In one aspect, a large number of kinase substrates are assayed in what is termed a “comprehensive kinase survey.” Selective mTOR inhibitors are expected to inhibit phosphorylation of mTOR substrates without inhibiting phosphorylation of the substrates of other kinases. Alternatively, selective mTOR inhibitors may inhibit phosphorylation of substrates of other kinases through anticipated or unanticipated mechanisms such as feedback loops or redundancy.

Effect of inhibition of mTORC1 and/or mTORC2 can be established by cell colony formation assay or other forms of cell proliferation assay. A wide range of cell proliferation assays are available in the art, and many of which are available as kits. Non-limiting examples of cell proliferation assays include testing for tritiated thymidine uptake assays, BrdU (5′-bromo-2′-deoxyuridine) uptake (kit marketed by Calibochem), MTS uptake (kit marketed by Promega), MTT uptake (kit marketed by Cayman Chemical), CyQUANT® dye uptake (marketed by Invitrogen).

Apoptosis and cell cycle arrest analysis can be performed with any methods exemplified herein as well other methods known in the art. Many different methods have been devised to detect apoptosis. Exemplary assays include but are not limited to the TUNEL (TdT-mediated dUTP Nick-End Labeling) analysis, ISEL (in situ end labeling), and DNA laddering analysis for the detection of fragmentation of DNA in populations of cells or in individual cells, Annexin-V analysis that measures alterations in plasma membranes, detection of apoptosis related proteins such p53 and Fas.

A cell-based assay typically proceeds with exposing the target cells (e.g., in a culture medium) to a candidate mTORC1 and/or mTORC2 selective inhibitor, and then assaying for readout under investigation. Depending on the nature of the candidate compounds (e.g. mTOR inhibitor), they can directly be added to the cells or in conjunction with carriers. For instance, when the agent is nucleic acid, it can be added to the cell culture by methods well known in the art, which include without limitation calcium phosphate precipitation, microinjection or electroporation. Alternatively, the nucleic acid can be incorporated into an expression or insertion vector for incorporation into the cells. Vectors that contain both a promoter and a cloning site into which a polynucleotide can be operatively linked are well known in the art. Such vectors are capable of transcribing RNA in vitro or in vitro, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of vectors are viruses, such as baculovirus and retrovirus, bacteriophage, adenovirus, adeno-associated virus, cosmid, plasmid, fungal vectors and other recombination vehicles typically used in the art which have been described for expression in a variety of eukaryotic and prokaryotic hosts, and may be used for gene therapy as well as for simple protein expression. Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens. Liposomes that also comprise a targeting antibody or fragment thereof can be used in the methods of this invention. Other biologically acceptable carriers can be utilized, including those described in, for example, REMINGTON'S PHARMACEUTICAL SCIENCES, 19th Ed. (2000), in conjunction with the subject compounds.

The subject agents can also be utilized to inhibit phosphorylation of both Akt (S473) and Akt (T308) in a cell. Accordingly, the present invention provides for a method comprising the step of contacting a cell with an effective amount of such biologically active agent such that Akt phosphorylation at residues S473 and T308 is simultaneously inhibited. In one aspect, the biologically active agent inhibits phosphorylation of S473 of Akt more effectively than phosphorylation of T308 of Akt when tested at a comparable molar concentration, preferably at an identical molar concentration.

Inhibition of Akt phosphorylation can be determined using any methods known in the art or described herein. Representative assays include but are not limited to immunoblotting and immunoprecipitation with antibodies such as anti-phosphotyrosine antibodies that recognize the specific phosphorylated proteins. Cell-based ELISA kit quantifies the amount of activated (phosphorylated at S473) Akt relative to total Akt protein is also available (SuperArray Biosciences).

In practicing the subject methods, any cystic cells that express mTORC1, mTORC2 and/or Akt can be utilized. Non-limiting examples of specific cell types whose proliferation can be inhibited include cells of epithelial tissues (e.g. liver, kidney and pancreas). Also of interest are cells exhibiting a neoplastic propensity or phenotype. Of particular interest is the type of cells that differentially expresses (over-expresses or under-expresses) a polycystic disease-causing gene (e.g., PKD1 or PKD2). The types of autosomal polycystic disorders diseases involving abnormal functioning of genes include but are not limited to ADPKD, ARPKD, and ADPLD.

In some embodiments, the compound (e.g. mTOR inhibitor) inhibits both mTORC1 and mTORC2 with an IC₅₀ value of about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM or less as ascertained in an in vitro kinase assay, and said IC₅₀ value is at least 2, 3, 4, 5, 6, 7, 8, 9, 10, 15, 20, 25, 30, 35, 40, 45, 50, 100, or 1000 times less than its IC₅₀ value against all other type I PI3-kinases selected from the group consisting of PI3-kinase α, PI3-kinase β, PI3-kinase γ, and PI3-kinase δ. For example, the mTOR inhibitor inhibits both mTORC1 and mTORC2 with an IC₅₀ value of about 200, 100, 75, 50, 25, 10, 5, 1 or 0.5 nM or less as ascertained in an in vitro kinase assay. In one instance, the mTOR inhibitor inhibits both mTORC1 and mTORC2 with an IC₅₀ value of about 100 nM or less as ascertained in an in vitro kinase assay. Alternatively, the mTOR inhibitor inhibits both mTORC1 and mTORC2 with an IC₅₀ value of about 10 nM or less as ascertained in an in vitro kinase assay.

In some embodiments, the present invention provides the use of a compound (e.g. mTOR inhibitor), wherein the compound (e.g. mTOR inhibitor) directly binds to and inhibits both mTORC1 and mTORC2 with an IC₅₀ value of about or less than a predetermined value, as ascertained in an in vitro kinase assay. In some embodiments, the compound (e.g. mTOR inhibitor) inhibits both mTORC1 and mTORC2 with an IC₅₀ value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM or less, 60 μM or less, 70 μM or less, 80 μM or less, 90 μM or less, 100 μM or less, 200 μM or less, 300 μM or less, 400 μM or less, or 500 μM or less.

In some embodiments, the compound (e.g. mTOR inhibitor) inhibits both mTORC1 and mTORC2 with an IC₅₀ value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM or less, 60 μM or less, 70 μM or less, 80 μM or less, 90 μM or less, 100 μM or less, 200 μM or less, 300 μM or less, 400 μM or less, or 500 μM or less, and the compound (e.g. mTOR inhibitor) is substantially inactive against one or more types I PI3-kinases selected from the group consisting of PI3-kinase α, PI3-kinase β, PI3-kinase γ, and PI3-kinase δ. In some embodiments, the compound (e.g. mTOR inhibitor) inhibits both mTORC1 and mTORC2 with an IC₅₀ value of about 10 nM or less as ascertained in an in vitro kinase assay, and the compound (e.g. mTOR inhibitor) is substantially inactive against one or more types I PI3-kinases selected from the group consisting of PI3-kinase α, PI3-kinase β, PI3-kinase γ, and PI3-kinase δ.

As used herein, the terms “substantially inactive” refers to an inhibitor that inhibits the activity of its target by less than approximately 1%, 5%, 10%, 15% or 20% of its maximal activity in the absence of the inhibitor, as determined by an in vitro enzymatic assay (e.g. in vitro kinase assay).

In other embodiments, the compound (e.g. mTOR inhibitor) inhibits both mTORC1 and mTORC2 with an IC₅₀ value of about 1000, 500, 100, 75, 50, 25, 10, 5, 1, or 0.5 nM or less as ascertained in an in vitro kinase assay, and said IC₅₀ value is at least 2, 5, 10, 15, 20, 50, 100 or 100 times less than its IC₅₀ value against all other type I PI3-kinases selected from the group consisting of PI3-kinase α, PI3-kinase β, PI3 kinase γ, and PI3-kinase δ. For example, the compound (e.g. mTOR inhibitor) inhibits both mTORC1 and mTORC2 with an IC₅₀ value of about 100 nM or less as ascertained in an in vitro kinase assay, and said IC₅₀ value is at least 5 times less than its IC₅₀ value against all other type I PI3-kinases selected from the group consisting of PI3-kinase α, PI3-kinase β, PI3-kinase γ, and PI3-kinase δ.

In some embodiments, the compound (e.g. mTOR inhibitor) inhibits both mTORC1 and mTORC2 with an IC₅₀ value of about 100 nM or less as ascertained in an in vitro kinase assay, and said IC₅₀ value is at least 5 times less than its IC₅₀ value against all other type I PI3-kinases selected from the group consisting of PI3-kinase α, PI3-kinase β, PI3-kinase γ, and PI3-kinase δ.

Compounds (e.g. mTOR inhibitor) suitable for use in the subject methods are selected from a variety types of molecules. For example, an inhibitor can be biological or chemical compound such as a simple or complex organic or inorganic molecule, peptide, peptide mimetic, protein (e.g. antibody), liposome, or a polynucleotide (e.g. small interfering RNA, microRNA, anti-sense, aptamer, ribozyme, or triple helix). Some exemplary classes of chemical compounds suitable for use in the subject methods are detailed in the sections below.

The advantages of selective inhibition of a cellular target as a way of treating a disease condition mediated by such target are manifold. For example, in the case of PKD, because healthy cells depend on the same signaling pathways that are activated in the case of PKD, inhibition of these pathways during disease treatment can cause harmful side effects. In order for a method of treating an autosomal polycystic disorder, such as PKD, to be successful without causing excessive damage to healthy cells, a very high degree of specificity in targeting the aberrant signaling component or components is desirable.

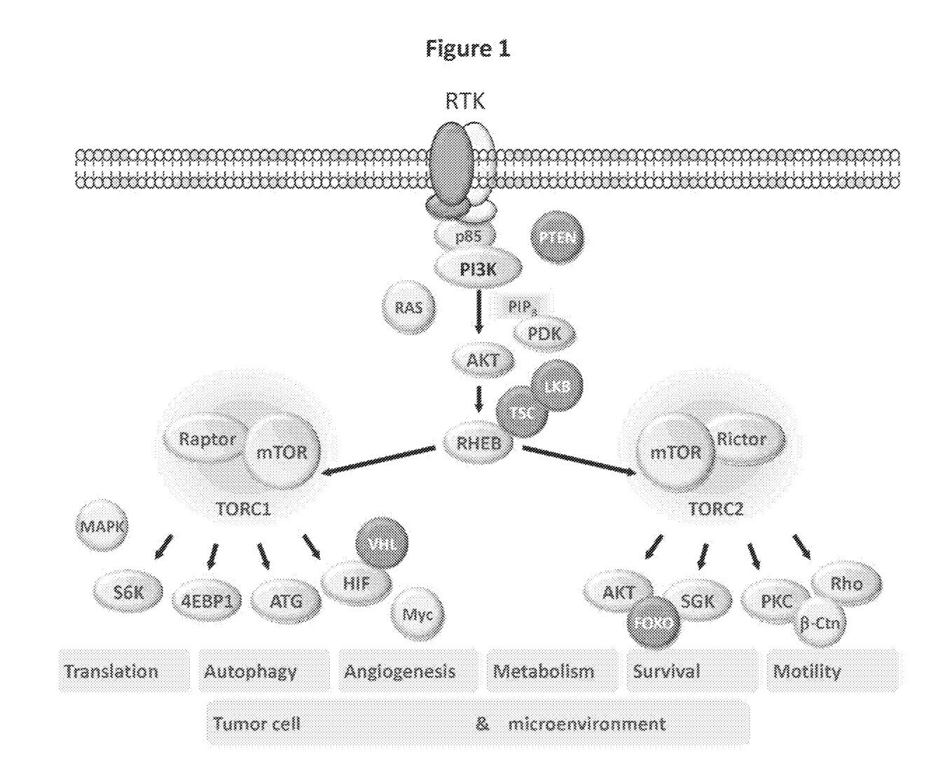

Some of the signaling pathways that contain mTOR are illustrated in FIG. 1. One major downstream effector of mTOR signaling is the Akt serine/threonine kinase. Akt possesses a protein domain known as a PH domain, or Pleckstrin Homology domain, which binds to phosphoinositides with high affinity. In the case of the PH domain of Akt, it binds either PIP3 (phosphatidylinositol (3,4,5)-trisphosphate, PtdIns(3,4,5)P3) or PIP2 (phosphatidylinositol (3,4)-bisphosphate, PtdIns(3,4)P2). PI3K phosphorylates PIP2 in response to signals from chemical messengers, such as ligand binding to G protein-coupled receptors or receptor tyrosine kinases. Phosphorylation by PI3K converts PIP2 to PIP3, recruiting Akt to the cell membrane where it is phosphorylated at serine 473 (S473) by mTORC2. Phosphorylation of Akt at another site, threonine 308 (T308), is not directly dependent on mTORC2, but requires PI3K activity. Therefore, PI3K activity towards Akt can be isolated from mTOR activity by examining Akt threonine 308 phosphorylation status in cells lacking mTORC2 activity.

The subject methods are useful for treating a polycystic kidney disease condition associated with mTOR.

The data presented in the Examples herein below demonstrate that compounds (e.g. mTOR inhibitor) of the present invention is useful for treating polycystic kidney disorder. Non-limiting examples of such conditions include but are not limited to ADPKD, ARPKD, or any combination thereof.

Certain embodiments contemplate a human subject such as a subject that has been diagnosed as having or being at risk for developing or acquiring an autosomal polycystic disease condition (e.g., PKD) associated with mTOR. Certain other embodiments contemplate a non-human subject, for example a non-human primate such as a macaque, chimpanzee, gorilla, vervet, orangutan, baboon or other non-human primate, including such non-human subjects that can be known to the art as preclinical models, including preclinical models for autosomal polycystic disorders (e.g., PKD). Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of the present invention, a transgenic animal is utilized. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells).

In one aspect, a compound of Formula (I) is provided:

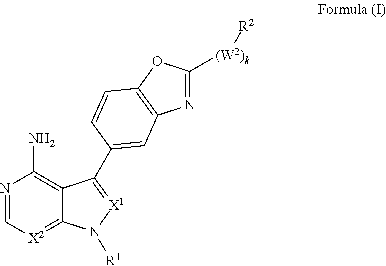

wherein:

### X¹ is N or C-E¹;

### X² is N or CH;

E¹ is —(W¹)ⱼ—R⁴;
W¹ is —O—, —NR^{7A}—, —S(O)₀₋₂, —CO—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A}), —CH(R^{7A})N(SO₂R^{8A}), —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—; W² is —O—, —NR⁷—, —S(O)₀₋₂—, —C(O)—, —C(O)N(R⁷)—, —(N)(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is —H, -aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl, each of which, except for —H, is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl (e.g. bicyclic aryl, unsubstituted aryl, or substituted monocyclic aryl), heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl moiety, wherein each of said moieties is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —O-aryl or —SC(═O)NR³¹R³²;
R³ and R⁴ are independently hydrogen, halogen, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl (e.g. bicyclic aryl, unsubstituted aryl, or substituted monocyclic aryl), heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl moiety, wherein each of said moieties is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³²;
each of R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or C₁₋₁₀alkyl, C₂₋₁₀alkenyl, C₂₋₁₀alkynyl, C₃₋₈cycloalkyl, heteroalkyl, aryl, heteroaryl, or heterocyclyl moiety, wherein each of said moieties is unsubstituted or is substituted with one or more C₁₋₁₀alkyl, C₂₋₁₀alkenyl, C₂₋₁₀alkynyl, C₃₋₁₀cycloalkyl, heteroalkyl, aryl, heteroaryl, heterocyclyl substituent, wherein each of said substituents is unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂ NH(C₁₋₁₀alkyl), —COOH, or —SO₂NR³⁴R³⁵;
R³⁴ and R³⁵ in —NR³⁴R³⁵, —C(═O)NR³⁴R³⁵, or —SO₂NR³⁴R³⁵, are taken together with the nitrogen atom to which they are attached to form a 3-10 membered saturated or unsaturated ring; wherein said ring is independently unsubstituted or is substituted by one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵, and wherein said 3-10 membered saturated or unsaturated ring independently contains 0, 1, or 2 more heteroatoms in addition to the nitrogen atom;
each of R⁷, R^{7A}, R⁸ and R^{8A} is independently hydrogen, C₁₋₁₀alkyl, C₂₋₁₀alkenyl, C₂₋₁₀alkynyl, aryl, heteroalkyl, heteroaryl, heterocyclyl or C₃₋₁₀cycloalkyl, each of which except for hydrogen is unsubstituted or is substituted by one or more independent R⁶ substituents; and
R⁶ is halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³²; or C₁₋₁₀alkyl, C₂₋₁₀alkenyl, C₂₋₁₀alkynyl, C₃₋₈cycloalkyl, heteroalkyl, aryl, heteroaryl, heterocyclyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵. In some embodiments, the compound is an mTOR inhibitor.

Within each aspect and each embodiment:

Each R⁴ in a compound may be independently different. Each R⁵ in a compound may be independently different. Each R⁶ in a compound may be independently different. Each R⁷ in a compound may be independently different. Each R^{7A} in a compound may be independently different. Each R⁸ in a compound may be independently different. Each R^{8A} in a compound may be independently different. Each R³¹ in a compound may be independently different. Each R³² in a compound may be independently different. Each R³³ in a compound may be independently different. Each R³⁴ in a compound may be independently different. Each R³⁵ in a compound may be independently different. For example, a compound comprising and R⁶ substituted R^{7A} and a R⁶ substituted R⁷ may have a particular R⁶ (e.g. C1alkyl) on R^{7A} and a different R⁶ on R⁷ (e.g. phenyl). Furthermore, each occurrence of a moiety such as C₁₋₁₀alkyl, which encompasses multiple groups may each be a different member of that group (e.g. one a methyl and another an ethyl).

In a second aspect, a compound of Formula (I) is provided

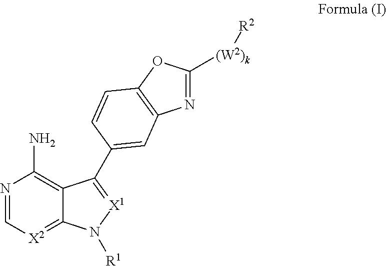

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is hydrogen, R³-substituted or unsubstituted C₁₋₁₀alkyl, R³-substituted or unsubstituted C₂₋₁₀alkenyl, R³-substituted or unsubstituted C₂₋₁₀alkynyl, R³-substituted or unsubstituted C₃₋₈cycloalkyl, R³-substituted or unsubstituted C₃₋₈cycloalkenyl, R³-substituted or unsubstituted C₃₋₈cycloalkynyl, R³-substituted or unsubstituted heteroalkyl, R³-substituted or unsubstituted heteroalkenyl, R³-substituted or unsubstituted heteroalkynyl, R³-substituted or unsubstituted heterocyclyl, R³-substituted or unsubstituted aryl, R³-substituted or unsubstituted heteroaryl; wherein each R³-substituted R¹ is independently substituted with one or more R³
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R² is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).
R³ and R⁴ are independently is hydrogen, oxo, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R³ or R⁴ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, C(═O)NR³¹R³², C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl);
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C_{—10}alkyl), —SO₂NH(C₁₋₁₀alkyl), —COOH, or —SO₂NR³⁴R³⁵;
each R⁷, R^{7A}, R⁸ and R^{8A} is independently hydrogen, R⁶-substituted or unsubstituted C₁₋₁₀alkyl, R⁶-substituted or unsubstituted C₂₋₁₀alkenyl, R⁶-substituted or unsubstituted C₂₋₁₀alkynyl, R⁶-substituted or unsubstituted C₃₋₈cycloalkyl, R⁶-substituted or unsubstituted C₃₋₈cycloalkenyl, R⁶-substituted or unsubstituted C₃₋₈cycloalkynyl, R⁶-substituted or unsubstituted heteroalkyl, R⁶-substituted or unsubstituted heteroalkenyl, R⁶-substituted or unsubstituted heteroalkynyl, R⁶-substituted or unsubstituted heterocyclyl, R⁶-substituted or unsubstituted aryl, R⁶-substituted or unsubstituted heteroaryl; wherein each R⁶-substituted R⁷, R^{7A}, R⁸ and R^{8A} is independently substituted with one or more R⁶; and
R⁶ is independently halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R⁶ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl). In some embodiments, the compound is an mTOR inhibitor.

Within each aspect and each embodiment:

Each R⁴ in a compound may be independently different. Each R⁵ in a compound may be independently different. Each R⁶ in a compound may be independently different. Each R⁷ in a compound may be independently different. Each R^{7A} in a compound may be independently different. Each R⁸ in a compound may be independently different. Each R^{8A} in a compound may be independently different. Each R³¹ in a compound may be independently different. Each R³² in a compound may be independently different. Each R³³ in a compound may be independently different. Each R³⁴ in a compound may be independently different. Each R³⁵ in a compound may be independently different. For example, a compound comprising and R⁶ substituted R^{7A} and a R⁶ substituted R⁷ may have a particular R⁶ (e.g. C1alkyl) on R^{7A} and a different R⁶ on R⁷ (e.g. phenyl). Furthermore, each occurrence of a moiety such as C₁₋₁₀alkyl, which encompasses multiple groups may each be a different member of that group (e.g. one a methyl and another an ethyl).

In a third aspect, a compound of Formula (I) is provided:

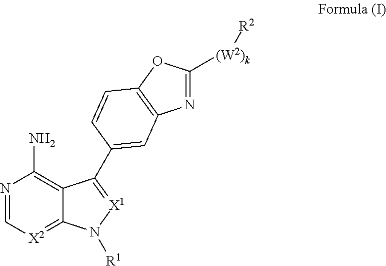

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—.

### W² is —O—, —NR⁷—, —S(O)₀₋₂—, —C(O)—, —C(O)N(R⁷)—, —N(R⁷)C(O)—, or —N(R⁷)C(O)N(R⁸)—;

j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹R³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C_{—10}alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸ and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents; and
R⁶ is halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

Within each aspect and each embodiment:

Each R⁴ in a compound may be independently different. Each R⁵ in a compound may be independently different. Each R⁶ in a compound may be independently different. Each R⁷ in a compound may be independently different. Each R^{7A} in a compound may be independently different. Each R⁸ in a compound may be independently different. Each R^{8A} in a compound may be independently different. Each R³¹ in a compound may be independently different. Each R³² in a compound may be independently different. Each R³³ in a compound may be independently different. Each R³⁴ in a compound may be independently different. Each R³⁵ in a compound may be independently different. For example, a compound comprising and R⁶ substituted R^{7A} and a R⁶ substituted R⁷ may have a particular R⁶ (e.g. C1alkyl) on R^{7A} and a different R⁶ on R⁷ (e.g. phenyl). Furthermore, each occurrence of a moiety such as C₁₋₁₀alkyl, which encompasses multiple groups may each be a different member of that group (e.g. one a methyl and another an ethyl).

In certain embodiments of Formula I, X¹ is N. In other embodiments of Formula I, X¹ is C-E¹.

In certain embodiments of Formula I, X² is N. In other embodiments of Formula I, X² is CH.

In certain embodiments, R¹ is —C₁₋₁₀alkyl, which is unsubstituted. In other embodiments, R¹ is —C₁₋₁₀alkyl, which is substituted by one or more independent R³. In other embodiments, R¹ is —C₃₋₈cycloalkyl, which is unsubstituted. In another embodiment, R¹ is —C₃₋₈cycloalkyl, which is substituted by one or more independent R³. In some embodiments, R₁ is -L-C₁₋₁₀alkyl-C₃₋₈cycloalkyl, which is unsubstituted. In other embodiments, R₁ is -L-C₁₋₁₀alkyl-C₃₋₈cycloalkyl, which is substituted by one or more independent R³. In other embodiments, R₁ is heterocyclyl, which is substituted by one or more independent R³. In other embodiments, R₁ is heterocyclyl, which is unsubstituted.

For example, in some embodiments R¹ is one of the following groups:

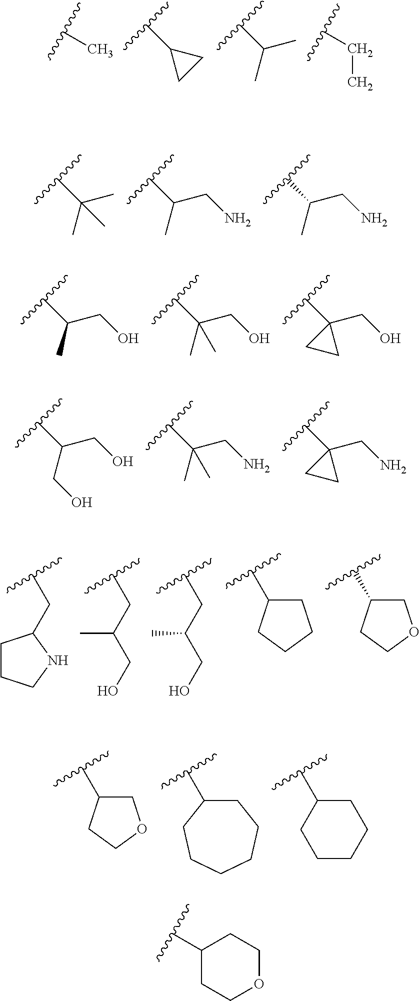

In some embodiments of the compound of Formula I, R² is hydrogen. In another embodiment, R² is halogen. In another embodiment, R² is —OH. In another embodiment, R² is —R³¹. In another embodiment, R² is —CF₃. In another embodiment, R² is —OCF₃. In another embodiment, R² is —OR³¹. In another embodiment, R² is —NR³¹R³². In another embodiment, R² is —NH₂. In another embodiment, R² is —NHC(O)CH₃. In another embodiment, R² is —NR³⁴R³⁵. In another embodiment, R² is —C(O)R³¹. In another embodiment, R² is —CO₂R³¹. In another embodiment, R² is —C(═O)NR³¹R³². In another embodiment, R² is —C(═O)NR³⁴R³⁵. In another embodiment, R² is —NO₂. In another embodiment, R² is —CN. In another embodiment, R² is —S(O)₀₋₂R³. In another embodiment, R² is —SO₂NR³¹R³². In another embodiment, R² is —SO₂NR³⁴R³⁵. In another embodiment, R² is —NR³¹C(═O)R³². In another embodiment, R² is —NR³¹C(═O)OR³². In another embodiment, R² is —NR³¹C(═O)NR³²R³³. In another embodiment, R² is —NR³¹S(O)₀₋₂R³². In another embodiment, R² is —C(═S)OR³¹. In another embodiment, R² is —C(═O)SR³¹. In another embodiment, R² is —NR³¹C(═NR³²)NR³³R³². In another embodiment, R² is —NR³¹C(═NR³²)OR³³. In another embodiment, R² is —NR³¹C(═NR³²)SR³³. In another embodiment, R² is —OC(═O)OR³³. In another embodiment, R² is —OC(═O)NR³¹R³². In another embodiment, R² is —OC(═O)SR³¹. In another embodiment, R² is —SC(═O)OR³¹. In another embodiment, R² is —P(O)OR³¹OR³². In another embodiment, R² is —SC(═O)NR³¹R³². In another embodiment, R² is monocyclic aryl. In another embodiment, R² is bicyclic aryl. In another embodiment, R² is substituted monocyclic aryl. In another embodiment, R² is heteroaryl. In another embodiment, R² is C₁₋₄alkyl. In another embodiment, R² is C₁₋₁₀alkyl. In another embodiment, R² is C₃₋₈cycloalkyl. In another embodiment, R² is C₃₋₈cycloalkyl-C₁₋₁₀alkyl. In another embodiment, R² is C₁₋₁₀alkyl-C₃₋₈cycloalkyl. In another embodiment, R² is C₁₋₁₀alkyl-monocyclic aryl. In another embodiment, R² is C₂₋₁₀alkyl-monocyclic aryl. In another embodiment, R² is monocyclic aryl-C₂₋₁₀alkyl. In another embodiment, R² is C₁₋₁₀alkyl-bicyclicaryl. In another embodiment, R² is bicyclicaryl-C₁₋₁₀alkyl. In another embodiment, R² is —C₁₋₁₀alkylheteroaryl. In another embodiment, R² is —C₁₋₁₀alkylheterocyclyl. In another embodiment, R² is —C₂₋₁₀alkenyl. In another embodiment, R² is —C₂₋₁₀alkynyl. In another embodiment, R² is C₂₋₁₀alkenylaryl. In another embodiment, R² is C₂₋₁₀alkenylheteroaryl. In another embodiment, R² is C₂₋₁₀alkenylheteroalkyl. In another embodiment, R² is C₂₋₁₀alkenylheterocyclcyl. In another embodiment, R² is —C₂₋₁₀alkynylaryl. In another embodiment, R² is —C₂₋₁₀alkynylheteroaryl. In another embodiment, R² is —C₂₋₁₀alkynylheteroalkyl. In another embodiment, R² is —C₂₋₁₀alkynylheterocyclyl. In another embodiment, R² is —C₂₋₁₀alkynylC₃₋₈cycloalkyl. In another embodiment, R² is —C₂₋₁₀alkynylC₃₋₈cycloalkenyl. In another embodiment, R² is —C₁₋₁₀alkoxy-C₁₋₁₀alkyl. In another embodiment, R² is —C₁₋₁₀alkoxy-C₂₋₁₀alkenyl. In another embodiment, R² is —C₁₋₁₀alkoxy-C₂₋₁₀alkynyl. In another embodiment, R² is -heterocyclyl C₁₋₁₀alkyl. In another embodiment, R² is heterocyclylC₂₋₁₀alkenyl. In another embodiment, R² is heterocyclylC₂₋₁₀alkynyl. In another embodiment, R² is aryl-C₂₋₁₀alkyl. In another embodiment, R² is aryl-C₁₋₁₀alkyl. In another embodiment, R² is aryl-C₂₋₁₀alkenyl. In another embodiment, R² is aryl-C₂₋₁₀alkynyl. In another embodiment, R² is aryl-heterocyclyl. In another embodiment, R² is heteroaryl-C₁₋₁₀alkyl. In another embodiment, R² is heteroaryl-C₂₋₁₀alkenyl. In another embodiment, R² is heteroaryl-C₂₋₁₀alkynyl. In another embodiment, R² is heteroaryl-C₃₋₈cycloalkyl. In another embodiment, R² is heteroaryl-heteroalkyl. In another embodiment, R² is heteroaryl-heterocyclyl.

In certain embodiments of the compound of Formula (I), R³ is hydrogen. In another embodiment, R³ is halogen. In another embodiment, R³ is —OH. In another embodiment, R³ is —R³¹. In another embodiment, R³ is —CF₃. In another embodiment, R³ is —OCF₃. In another embodiment, R³ is —OR³¹. In another embodiment, R³ is —NR³¹R³². In another embodiment, R³ is —NR³⁴R³⁵. In another embodiment, R³ is —C(O)R³¹. In another embodiment, R³ is —CO₂R³¹. In another embodiment, R³ is —C(═O)NR³¹R³². In another embodiment, R³ is —C(═O)NR³⁴R³⁵. In another embodiment, R³ is —NO₂. In another embodiment, R³ is —CN. In another embodiment, R³ is —S(O)₀₋₂R³. In another embodiment, R³ is —SO₂NR³¹R³². In another embodiment, R³ is —SO₂NR³⁴R³⁵. In another embodiment, R³ is —NR³¹C(═O)R³². In another embodiment, R³ is —NR³¹C(═O)OR³². In another embodiment, R³ is —NR³¹C(═O)NR³²R³³. In another embodiment, R³ is —NR³¹S(O)₀₋₂R³². In another embodiment, R³ is —C(═S)OR³¹. In another embodiment, R³ is —C(═O)SR³¹. In another embodiment, R³ is —NR³¹C(═NR³²)NR³³R³². In another embodiment, R³ is —NR³¹C(═NR³²)OR³³. In another embodiment, R³ is —NR³¹C(═NR³²)SR³³. In another embodiment, R³ is —OC(═O)OR³³. In another embodiment, R³ is —OC(═O)NR³¹R³². In another embodiment, R³ is —OC(═O)SR³¹. In another embodiment, R³ is —SC(═O)OR³¹. In another embodiment, R³ is —P(O)OR³¹OR³². In another embodiment, R³ is —SC(═O)NR³¹R³². In another embodiment, R³ is aryl. In another embodiment, R² is heteroaryl. In another embodiment, R³ is C₁₋₄alkyl. In another embodiment, R³ is C₁₋₁₀alkyl. In another embodiment, R³ is C₃₋₈cycloalkyl. In another embodiment, R³ is C₃₋₈cycloalkyl-C₁₋₁₀alkyl. In another embodiment, R³ is —C₁₋₁₀alkyl-C₃₋₈cycloalkyl. In another embodiment, R³ is C₂₋₁₀alkyl-monocyclic aryl. In another embodiment, R³ is monocyclic aryl-C₂₋₁₀alkyl. In another embodiment, R³ is C₁₋₁₀alkyl-bicyclicaryl. In another embodiment, R³ is bicyclicaryl-C₁₋₁₀alkyl. In another embodiment, R³ is C₁₋₁₀alkylheteroaryl. In another embodiment, R³ is C₁₋₁₀alkylheterocyclyl. In another embodiment, R³ is C₂₋₁₀alkenyl. In another embodiment, R³ is C₂₋₁₀alkynyl. In another embodiment, R³ is C₂₋₁₀alkenylaryl. In another embodiment, R³ is C₂₋₁₀alkenylheteroaryl. In another embodiment, R³ is C₂₋₁₀alkenylheteroalkyl. In another embodiment, R³ is C₂₋₁₀alkenylheterocyclcyl. In another embodiment, R³ is —C₂₋₁₀alkynylaryl. In another embodiment, R³ is —C₂₋₁₀alkynylheteroaryl. In another embodiment, R³ is —C₂₋₁₀alkynylheteroalkyl. In another embodiment, R³ is C₂₋₁₀alkynylheterocyclyl. In another embodiment, R³ is —C₂₋₁₀alkynylC₃₋₈cycloalkyl. In another embodiment, R³ is C₂₋₁₀alkynylC₃₋₈cycloalkenyl. In another embodiment, R³ is —C₁₋₁₀alkoxy-C₁₋₁₀alkyl. In another embodiment, R³ is C₁₋₁₀alkoxy-C₂₋₁₀alkenyl. In another embodiment, R³ is —C₁₋₁₀alkoxy-C₂₋₁₀alkynyl. In another embodiment, R³ is heterocyclyl-C₁₋₁₀alkyl. In another embodiment, R³ is -heterocyclylC₂₋₁₀alkenyl. In another embodiment, R³ is heterocyclyl-C₂₋₁₀alkynyl. In another embodiment, R³ is aryl-C₁₋₁₀alkyl. In another embodiment, R³ is aryl-C₂₋₁₀alkenyl. In another embodiment, R³ is aryl-C₂₋₁₀alkynyl. In another embodiment, R³ is aryl-heterocyclyl. In another embodiment, R³ is heteroaryl-C₁₋₁₀alkyl. In another embodiment, R³ is heteroaryl-C₂₋₁₀alkenyl. In another embodiment, R³ is heteroaryl-C₂₋₁₀alkynyl. In another embodiment, R³ is heteroaryl-C₃₋₈cycloalkyl. In another embodiment, R³ is heteroaryl-heteroalkyl. In another embodiment, R³ is heteroaryl-heterocyclyl.

In certain embodiments of the compound of Formula (I), R⁴ is hydrogen. In another embodiment, R⁴ is halogen. In another embodiment, R⁴ is —OH. In another embodiment, R⁴ is —R³¹. In another embodiment, R⁴ is —CF₃. In another embodiment, R⁴ is —OCF₃. In another embodiment, R⁴ is —OR³¹. In another embodiment, R⁴ is —NR³¹R³². In another embodiment, R⁴ is —NR³⁴R³⁵. In another embodiment, R⁴ is —C(O)R³¹. In another embodiment, R⁴ is —CO₂R³¹. In another embodiment, R⁴ is —C(═O)NR³¹R³². In another embodiment, R⁴ is —C(═O)NR³⁴R³⁵. In another embodiment, R⁴ is —NO₂. In another embodiment, R⁴ is —CN. In another embodiment, R⁴ is —S(O)₀₋₂R³. In another embodiment, R⁴ is —SO₂NR³¹R³². In another embodiment, R⁴ is —SO₂NR³⁴R³⁵. In another embodiment, R⁴ is —NR³¹C(═O)R³². In another embodiment, R⁴ is —NR³¹C(═O)OR³². In another embodiment, R⁴ is —NR³¹C(═O)NR³²R³³. In another embodiment, R⁴ is —NR³¹S(O)₀₋₂R³². In another embodiment, R⁴ is —C(═S)OR³¹. In another embodiment, R⁴ is —C(═O)SR³¹. In another embodiment, R⁴ is —NR³¹C(═NR³²)NR³³R³². In another embodiment, R⁴ is —NR³¹C(═NR³²)OR³³. In another embodiment, R⁴ is —NR³¹C(═NR³²)SR³³. In another embodiment, R⁴ is —OC(═O)OR³³. In another embodiment, R⁴ is —OC(═O)NR³¹R³². In another embodiment, R⁴ is —OC(═O)SR³¹. In another embodiment, R⁴ is —SC(═O)OR³¹. In another embodiment, R⁴ is —P(O)OR³¹OR³². In another embodiment, R⁴ is —SC(═O)NR³¹R³². In another embodiment, R⁴ is aryl. In another embodiment, R⁴ is heteroaryl. In another embodiment, R⁴ is C₁₋₄alkyl. In another embodiment, R⁴ is C₁₋₁₀alkyl. In another embodiment, R⁴ is C₃₋₈cycloalkyl. In another embodiment, R⁴ is C₁₋₁₀alkyl-C₃₋₈cycloalkyl. In another embodiment, R⁴ is C₁₋₁₀alkylaryl. In another embodiment, R⁴ is C₁₋₁₀alkylheteroaryl. In another embodiment, R⁴ is C₁₋₁₀alkylheterocyclyl. In another embodiment, R⁴ is C₂₋₁₀alkenyl. In another embodiment, R⁴ is C₂₋₁₀alkynyl. In another embodiment, R⁴ is C₂₋₁₀alkynyl-C₃₋₈cycloalkyl. R⁴ is C₂₋₁₀alkenyl-C₃₋₈cycloalkyl. In another embodiment, R⁴ is C₂₋₁₀alkenylaryl. In another embodiment, R⁴ is C₂₋₁₀alkenyl-heteroaryl. In another embodiment, R⁴ is C₂₋₁₀alkenylheteroalkyl. In another embodiment, R⁴ is C₂₋₁₀alkenylheterocyclcyl. In another embodiment, R⁴ is —C₂₋₁₀alkynylaryl. In another embodiment, R⁴ is C₂₋₁₀alkynylheteroaryl. In another embodiment, R⁴ is C₂₋₁₀alkynylheteroalkyl. In another embodiment, R⁴ is C₂₋₁₀alkynylheterocyclyl. In another embodiment, R⁴ is C₂₋₁₀alkynylC₃₋₈cycloalkyl. In another embodiment, R⁴ is heterocyclyl C₁₋₁₀alkyl. In another embodiment, R⁴ is heterocyclylC₂₋₁₀alkenyl. In another embodiment, R⁴ is heterocyclyl-C₂₋₁₀alkynyl. In another embodiment, R⁴ is aryl-C₁₋₁₀alkyl. In another embodiment, R⁴ is aryl-C₂₋₁₀alkenyl. In another embodiment, R⁴ is aryl-C₂₋₁₀alkynyl. In another embodiment, R⁴ is aryl-heterocyclyl. In another embodiment, R⁴ is heteroaryl-C₁₋₁₀alkyl. In another embodiment, R⁴ is heteroaryl-C₂₋₁₀alkenyl. In another embodiment, R⁴ is heteroaryl-C₂₋₁₀alkynyl. In another embodiment, R⁴ is C₃₋₈cycloalkyl-C₁₋₁₀alkyl. In another embodiment, R⁴ is C₃₋₈cycloalkyl-C₂₋₁₀alkenyl. In another embodiment, R⁴ is C₃₋₈cycloalkyl-C₂₋₁₀alkynyl.

In certain embodiments of the compound of Formula (I), X is C-E¹, where R⁴ is hydrogen and j is 0. In certain embodiments of the compound of Formula (I), each of R⁷, R⁸, R^{7A} and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents. In certain embodiments of the compound of Formula (I), each of R⁷, R⁸, R^{7A} and R^{8A} is independently hydrogen, C₁₋₁₀alkyl, C₂₋₁₀alkenyl, aryl, heteroaryl, heterocyclyl or C₃₋₁₀cycloalkyl, each of which except for hydrogen is unsubstituted or is substituted by one or more independent R⁶ substituents.

In certain embodiments of the compound of Formula (I), R⁷ is hydrogen. In another embodiment, R⁷ is unsubstituted C₁₋₁₀alkyl. In another embodiment, R⁷ is unsubstituted C₂₋₁₀alkenyl. In another embodiment, R⁷ is unsubstituted aryl. In another embodiment, R⁷ is unsubstituted heteroaryl. In another embodiment, R⁷ is unsubstituted heterocyclyl. In another embodiment, R⁷ is unsubstituted C₃₋₁₀cycloalkyl. In another embodiment, R⁷ is C₁₋₁₀alkyl substituted by one or more independent R⁶. In another embodiment, R⁷ is C₂₋₁₀alkenyl substituted by one or more independent R⁶. In another embodiment, R⁷ is aryl substituted by one or more independent R⁶. In another embodiment, R⁷ is heteroaryl substituted by one or more independent R⁶. In another embodiment, R⁷ is heterocycly substituted by one or more independent R⁶. In another embodiment, R⁷ is C₃₋₁₀cycloalkyl substituted by one or more independent R⁶.

In certain embodiments of the compound of Formula (I), R^{7A} is hydrogen. In another embodiment, R^{7A} is unsubstituted C₁₋₁₀alkyl. In another embodiment, R^{7A} is unsubstituted C₂₋₁₀alkenyl. In another embodiment, R^{7A} is unsubstituted aryl. In another embodiment, R^{7A} is unsubstituted heteroaryl. In another embodiment, R^{7A} is unsubstituted heterocyclyl. In another embodiment, R^{7A} is unsubstituted C₃₋₁₀cycloalkyl. In another embodiment, R^{7A} is C₁₋₁₀alkyl substituted by one or more independent R⁶. In another embodiment, R^{7A} is C₂₋₁₀alkenyl substituted by one or more independent R⁶. In another embodiment, R^{7A} is aryl substituted by one or more independent R⁶. In another embodiment, R^{7A} is heteroaryl substituted by one or more independent R⁶. In another embodiment, R^{7A} is heterocycly substituted by one or more independent R⁶. In another embodiment, R^{7A} is C₃₋₁₀cycloalkyl substituted by one or more independent R⁶.

In certain embodiments of the compound of Formula (I), R⁸ is hydrogen. In another embodiment, R⁸ is unsubstituted C₁₋₁₀alkyl. In another embodiment, R⁸ is unsubstituted C₂₋₁₀alkenyl. In another embodiment, R⁸ is unsubstituted aryl. In another embodiment, R⁸ is unsubstituted heteroaryl. In another embodiment, R⁸ is unsubstituted heterocyclyl. In another embodiment, R⁸ is unsubstituted C₃₋₁₀cycloalkyl. In another embodiment, R⁸ is C₁₋₁₀alkyl substituted by one or more independent R⁶. In another embodiment, R⁸ is C₂₋₁₀alkenyl substituted by one or more independent R⁶. In another embodiment, R⁸ is aryl substituted by one or more independent R⁶. In another embodiment, R⁸ is heteroaryl substituted by one or more independent R⁶. In another embodiment, R⁸ is heterocyclyl substituted by one or more independent R⁶. In another embodiment, R⁸ is C₃₋₁₀cycloalkyl substituted by one or more independent R⁶.

In certain embodiments of the compound of Formula (I), R^{8A} is hydrogen. In another embodiment, R^{8A} is unsubstituted C₁₋₁₀alkyl. In another embodiment, R^{8A} is unsubstituted C₂₋₁₀alkenyl. In another embodiment, R^{8A} is unsubstituted aryl. In another embodiment, R^{8A} is unsubstituted heteroaryl. In another embodiment, R^{8A} is unsubstituted heterocyclyl. In another embodiment, R^{8A} is unsubstituted C₃₋₁₀cycloalkyl. In another embodiment, R^{8A} is C₁₋₁₀alkyl substituted by one or more independent R⁶. In another embodiment, R^{8A} is C₂₋₁₀alkenyl substituted by one or more independent R⁶. In another embodiment, R^{8A} is aryl substituted by one or more independent R⁶. In another embodiment, R^{8A} is heteroaryl substituted by one or more independent R⁶. In another embodiment, R^{8A} is heterocyclyl substituted by one or more independent R⁶. In another embodiment, R^{8A} is C₃₋₁₀cycloalkyl substituted by one or more independent R⁶.

In some embodiments of the compound of Formula (I), k is 1. In other embodiments, k is 0.

In various embodiments of the compound of Formula (I), W² is —O—. In another embodiment, W² is —NR⁷—. In yet another embodiment, W² is —C(O)N(R⁷)—. In another embodiment, W² is —N(R⁷)C(O)—. In another embodiment, W² is —N(R⁷)C(O)N(R⁸)—. In yet another embodiment, W² is —N(R⁷)S(O)—. In still yet another embodiment, W² is —N(R⁷)S(O)₂—.

In certain embodiments of the compound of Formula (I), W² is —NR⁷—, where R⁷ is hydrogen, C₁₋₁₀alkyl, C₂₋₁₀alkenyl, aryl, heteroaryl, heterocyclyl or C₃₋₁₀cycloalkyl, each of which except for hydrogen is unsubstituted or is substituted by one or more independent R⁶ substituents. For example, R⁷ is hydrogen or unsubstituted C₁₋₁₀alkyl.

In various embodiments of the compounds described herein, X¹ is C—(W¹)ⱼ—R⁴. In various embodiments of X¹, j is 1, and W¹ is —O—. In various embodiments of X¹, j is 1, and W¹ is —NR^{7A}—. In various embodiments of X¹, j is 1, and W¹ is —NH—. In various embodiments of X¹, j is 1, and W¹ is —S(O)₀₋₂—. In various embodiments of X¹, j is 1, and W¹ is —C(O)—. In various embodiments of X¹, j is 1, and W¹ is C(O)N(R^{7A})—. In various embodiments of X¹, j is 1, and W¹ is —N(R^{7A})C(O)—. In various embodiments of X¹, j is 1, and W¹ is —N(R^{7A})S(O)—. In various embodiments of X¹, j is 1, and W¹ is —N(R^{7A})S(O)₂—. In various embodiments of X¹, j is 1, and W¹ is —C(O)O—. In various embodiments of X¹, j is 1, and W¹ is CH(R^{7A})N(C(O)OR^{8A})—. In various embodiments of X¹, j is 1, and W¹ is —CH(R^{7A})N(C(O)R^{8A})—. In various embodiments of X¹, j is 1, and W¹ is —CH(R^{7A})N(SO₂R^{8A})—. In various embodiments of X¹, j is 1, and W¹ is —CH(R^{7A})N(R^{8A})—. In various embodiments of X¹, j is 1, and W¹ is —CH(R^{7A})C(O)N(R^{8A})—. In various embodiments of X¹, j is 1, and W¹ is —CH(R^{7A})N(R^{8A})C(O)—. In various embodiments of X¹, j is 1, and W¹ is —CH(R^{7A})N(R^{8A})S(O)—. In various embodiments of X¹, j is 1, and W¹ is —CH(R^{7A})N(R^{8A})S(O)₂—. In some embodiments, W¹ is —O—, —NR^{7A}—, —S(O)₀₋₂—, —C(O)—, —C(O)N(R^{7A})C(O)—, or —N(R^{7A})C(O)N(R^{8A})—. In some embodiments of the compound of Formula (I), j is 1. In some embodiments of the compound of Formula (I), j is 0. In some embodiments of the compound of Formula (I), W¹ is —O—. In some embodiments of the compound of Formula (I), W¹ is —NR^{7A}—. In some embodiments of the compound of Formula (I), W¹ is —S(O)₀₋₂—. In some embodiments of the compound of Formula (I), W¹ is —C(O)—, —C(O)N(R^{7A})—. In some embodiments of the compound of Formula (I), W¹ is —N(R^{7A})C(O)—. In some embodiments of the compound of Formula (I), W¹ is —N(R^{7A})S(O)—. In some embodiments of the compound of Formula (I), W¹ is —N(R^{7A})S(O)₂—. In some embodiments of the compound of Formula (I), W¹ is —C(O)O—. In some embodiments of the compound of Formula (I), W¹ is —CH(R^{7A})N(C(O)OR^{8A})—. In some embodiments of the compound of Formula (I), W¹ is —CH(R^{7A})N(C(O)R^{8A})—. In some embodiments of the compound of Formula (I), W¹ is —CH(R^{7A})N(SO₂R^{8A})—. In some embodiments of the compound of Formula (I), W¹ is —CH(R^{7A})N(R^{8A})—. In some embodiments of the compound of Formula (I), W¹ is —CH(R^{7A})C(O)N(R^{8A})—. In some embodiments of the compound of Formula (I), W¹ is —CH(R^{7A})N(R^{8A})C(O)—. In some embodiments of the compound of Formula (I), W¹ is —CH(R^{7A})N(R^{8A})S(O)—. In some embodiments of the compound of Formula (I), W¹ is —CH(R^{7A})N(R^{8A})S(O)₂—.

In some embodiments of the compound of Formula (I), W² is —O—. In some embodiments of the compound of Formula (I), W² is —NR⁷—. In some embodiments of the compound of Formula (I), W² is —S(O)₀₋₂—. In some embodiments of the compound of Formula (I), W² is —C(O)—. In some embodiments of the compound of Formula (I), W² is —C(O)N(R⁷)—. In some embodiments of the compound of Formula (I), W² is —N(R⁷)C(O)—. In some embodiments of the compound of Formula (I), W² is —N(R⁷)S(O)—. In some embodiments of the compound of Formula (I), W² is —N(R⁷)S(O)₂—. In some embodiments of the compound of Formula (I), W² is —C(O)O—. In some embodiments of the compound of Formula (I), W² is —CH(R⁷)N(C(O)OR⁸)—. In some embodiments of the compound of Formula (I), W² is —CH(R⁷)N(C(O)R⁸)—. In some embodiments of the compound of Formula (I), W² is —CH(R⁷)N(SO₂R⁸)—. In some embodiments of the compound of Formula (I), W² is —CH(R⁷)N(R⁸)—. In some embodiments of the compound of Formula (I), W² is —CH(R⁷)C(O)N(R⁸)—. In some embodiments of the compound of Formula (I), W² is —CH(R⁷)N(R⁸)C(O)—. In some embodiments of the compound of Formula (I), W² is —CH(R⁷)N(R⁸)S(O)—. In some embodiments of the compound of Formula (I), W² is —CH(R⁷)N(R)S(O)₂—. In some embodiments of the compound of Formula (I), W² is —N(R⁷)C(O)N(R⁸)—.

In certain embodiments, the compound is a compound of Formula (Ia):

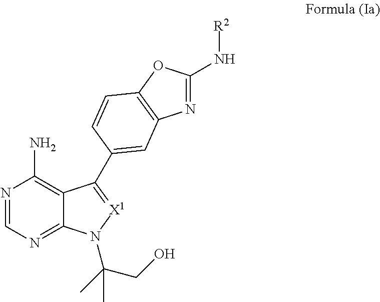

In certain embodiments, the compound is a compound of Formula (Ib):

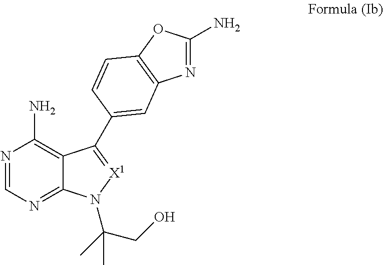

In some embodiments, the compound is Compound A:

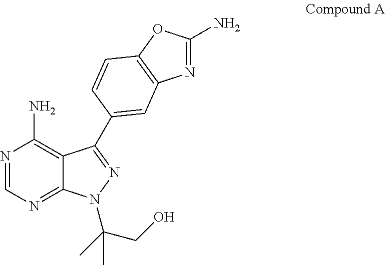 [SMILES: CC(C)(CO)n1nc(-c2ccc3oc(N)nc3c2)c2c(N)ncnc21]

In other embodiments, the compound is Compound B:

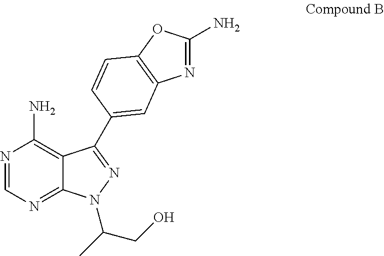 [SMILES: CC(CO)n1nc(-c2ccc3oc(N)nc3c2)c2c(N)ncnc21]

In other embodiments, the compound is Compound C:

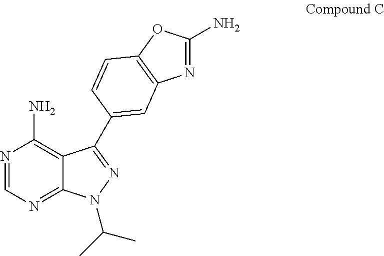 [SMILES: CC(C)n1nc(-c2ccc3oc(N)nc3c2)c2c(N)ncnc21]

In other embodiments, the compound is Compound D:

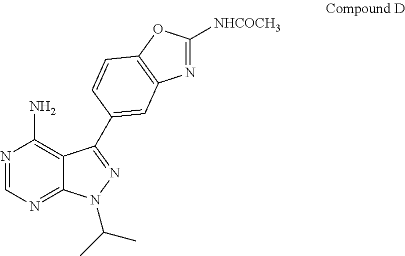

In certain embodiments, the compound is Compound E-1 or E-2:

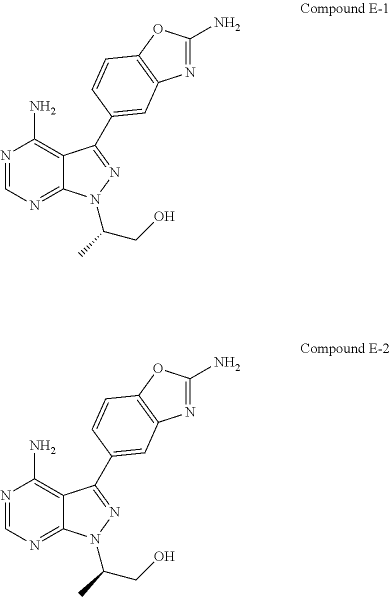 [SMILES: C[C@@H](CO)n1nc(-c2ccc3oc(N)nc3c2)c2c(N)ncnc21 ; C[C@H](CO)n1nc(-c2ccc3oc(N)nc3c2)c2c(N)ncnc21]

In other embodiments, the compound is Compound F:

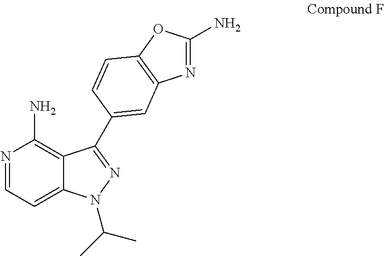 [SMILES: CC(C)n1nc(-c2ccc3oc(N)nc3c2)c2c(N)nccc21]

In still other embodiments, the compound is Compound G:

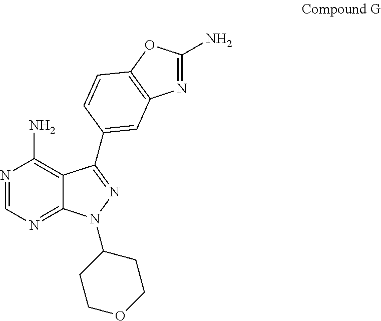 [SMILES: Nc1nc2cc(-c3nn(C4CCOCC4)c4ncnc(N)c34)ccc2o1]

Within each aspect and each embodiment above:

Each R⁴ in a compound may be independently different. Each R⁵ in a compound may be independently different. Each R⁶ in a compound may be independently different. Each R⁷ in a compound may be independently different. Each R^{7A} in a compound may be independently different. Each R⁸ in a compound may be independently different. Each R^{8A} in a compound may be independently different. Each R³¹ in a compound may be independently different. Each R³² in a compound may be independently different. Each R³³ in a compound may be independently different. Each R³⁴ in a compound may be independently different. Each R³⁵ in a compound may be independently different. For example, a compound comprising and R⁶ substituted R^{7A} and a R⁶ substituted R⁷ may have a particular R⁶ (e.g. C1alkyl) on R^{7A} and a different R⁶ on R⁷ (e.g. phenyl). Furthermore, each occurrence of a moiety such as C₁₋₁₀alkyl, which encompasses multiple groups may each be a different member of that group (e.g. one a methyl and another an ethyl).

In other embodiments, the compound (e.g. mTor inhibitor) is NVP-BEZ235 (Novartis), BGT226 (Novartis), XL765 (Sanofi-Aventis, Exelixis), GDC0980 (Genentech), SF1126 (Semafore), PKI587 (Wyeth), PF04691502 (Pfizer), or GSK2126458 (GlaxoSmithKline). In still other embodiments, the mTor inhibitor is CC223 (Celgene), OSI027 (OSI Pharmaceuticals), AZD8055 (Astra Zeneca), AZD2014 (Astra Zeneca), or Palomid 529 (Paloma Pharmaceuticals).

Structures of exemplary mTor inhibitors are shown in Table 1 below:

| mTor inhibitor | Structure |
|---|---|
| NVP-BEZ235 | 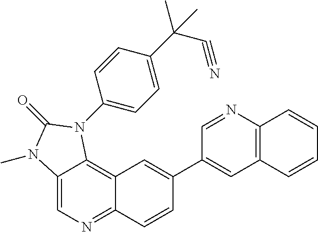 [SMILES: Cn1c(=O)n(-c2ccc(C(C)(C)C#N)cc2)c2c3cc(-c4cnc5ccccc5c4)ccc3ncc21] |
| XL765 | 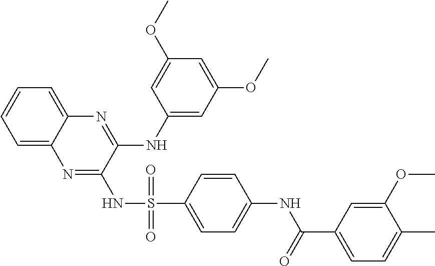 [SMILES: COc1cc(Nc2nc3ccccc3nc2NS(=O)(=O)c2ccc(NC(=O)c3ccc(C)c(OC)c3)cc2)cc(OC)c1] |
| GDC0980 | 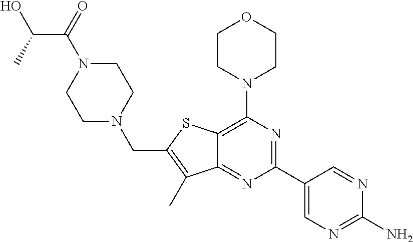 [SMILES: Cc1c(CN2CCN(C(=O)[C@H](C)O)CC2)sc2c(N3CCOCC3)nc(-c3cnc(N)nc3)nc12] |
| SF1126 | 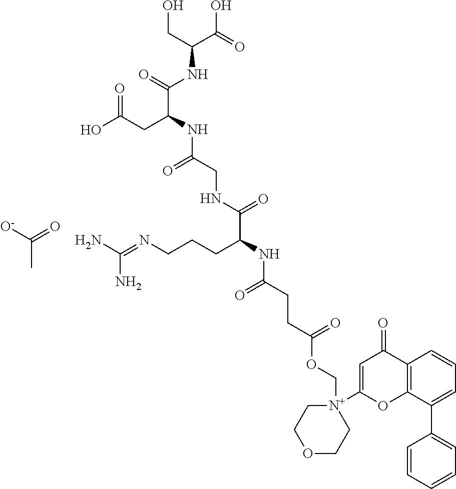 |
| PKI587 | 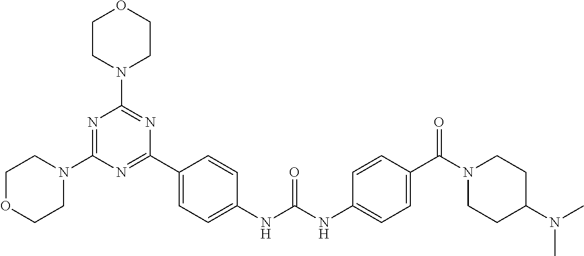 [SMILES: CN(C)C1CCN(C(=O)c2ccc(NC(=O)Nc3ccc(-c4nc(N5CCOCC5)nc(N5CCOCC5)n4)cc3)cc2)CC1] |
| PF04691502 | 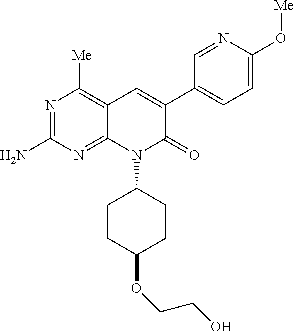 [SMILES: COc1ccc(-c2cc3c(C)nc(N)nc3n([C@H]3CC[C@H](OCCO)CC3)c2=O)cn1] |
| GSK2126458 | 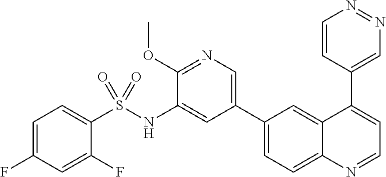 [SMILES: COc1ncc(-c2ccc3nccc(-c4ccnnc4)c3c2)cc1NS(=O)(=O)c1ccc(F)cc1F] |
| OSI027 | 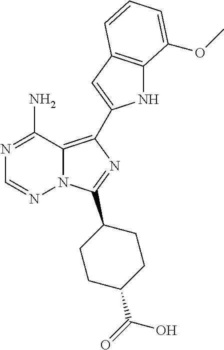 [SMILES: COc1cccc2cc(-c3nc([C@H]4CC[C@H](C(=O)O)CC4)n4ncnc(N)c34)[nH]c12] |
| AZD8055 | 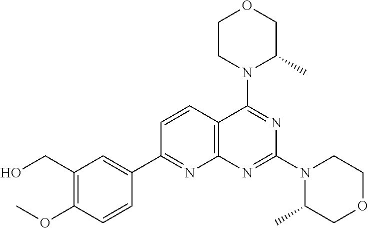 [SMILES: COc1ccc(-c2ccc3c(N4CCOC[C@@H]4C)nc(N4CCOC[C@@H]4C)nc3n2)cc1CO] |
| P529 | 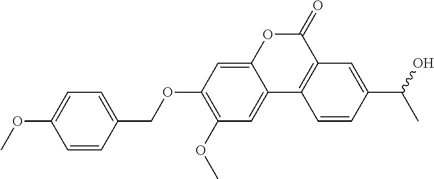 [SMILES: COc1ccc(COc2cc3oc(=O)c4cc(C(C)O)ccc4c3cc2OC)cc1] |

B. Treatment of Autosomal Polycystic Diseases

In an additional aspect, a method of treating polycystic kidney disease (PKD) in a subject in need thereof is provided, including administering to the subject a therapeutically effective amount of a compound as described herein (e.g. compound of Formula (I) including embodiments and aspects, Table 1).

In an additional aspect, a method of treating a polycystic disease in a subject in need thereof is provided, including administering to the subject a therapeutically effective amount of a compound as described herein (e.g. compound of Formula (I) including embodiments and aspects, Table 1). In some embodiments, of the method, the polycystic disease is polycystic kidney disease.

In an additional aspect, a method of inhibiting cyst formation in a subject at risk for developing PKD is provided, including contacting cyst cells with a compound as described herein (e.g. compound of Formula (I) including embodiments and aspects, Table 1) in an amount sufficient to inhibit growth of cyst cells. In some embodiments, the method further includes reducing cyst formation in an organ other than kidney.

In an additional aspect, a method is provide, including 1) evaluating whether a subject is susceptible to PKD, wherein the evaluation includes testing for (i) the presence of a biomarker correlated with PKD in the subject; and/or (ii) the presence of multiple kidney cysts; and 2) administering to the subject being tested for (a)(i) and/or (a)(ii) a pharmaceutical composition including an effective amount of a compound as described herein (e.g. compound of Formula (I) including embodiments and aspects, Table 1). In some embodiments of the method, the biomarker is a mutated PKD-1 or PKD-2 gene, or a respective gene product.

In one aspect, the invention provides for a method of treating an autosomal polycystic disease in a subject in need thereof, comprising administering to the subject a therapeutically effective amount of a compound provided herein (e.g. an mTOR inhibitor, a compound of Formula (I) (including embodiments and aspects), Table 1). In one embodiment, the autosomal polycystic disease is autosomal dominant polycystic kidney disease (ADPKD), autosomal recessive polycystic kidney disease (ARPKD) or autosomal dominant polycystic liver disease (ADPLD). In another aspect, a use of a compound as described herein (e.g. a compound of Formula (I), an mTOR inhibitor, compound of Table 1) in the manufacture of a medicament for the treatment of a disease (e.g. polycystic disease, polycystic kidney disease) is provided.

In one embodiment, the compound (e.g. mTOR inhibitor) contacts cyst cells and inhibits cyst formation and/or growth. Exemplary target cyst cells may be in affected tissues which include, but are not limited to, kidney, liver, pancreas or testes. In some embodiments, cyst formation and/or growth is reduced in the treated subject. For example, cyst formation and/or growth may be decreased by about 1-10%, 10-20%, 20-30%, 40-50%, 50-60%, 60-70%, 70-80%, 80-90%, or 90-100%. For example, cyst formation and/or growth may be decreased by 1-10%, 10-20%, 20-30%, 40-50%, 50-60%, 60-70%, 70-80%, 80-90%, or 90-100%. In some embodiments, cyst volume is reduced in the treated subject. For example, cyst volume may be reduced by about 1-10%, 10-20%, 20-30%, 40-50%, 50-60%, 60-70%, 70-80%, 80-90%, or 90-100%. For example, cyst volume may be reduced by 1-10%, 10-20%, 20-30%, 40-50%, 50-60%, 60-70%, 70-80%, 80-90%, or 90-100%. In the case of PKD, in addition to cyst formation, the disease has been associated with hypertension, increased renal mass, and reduced renal blood flow. In some embodiments, treatment with the compound (e.g. mTOR inhibitor) decreases hypertension in the subject. In some embodiments, treatment with the compound (e.g. mTOR inhibitor) decreases increased renal blood flow in the subject. In some embodiments, treatment with the compound (e.g. mTOR inhibitor) increases decreased renal blood flow in the subject. In some embodiments, treatment with the compound (e.g. mTOR inhibitor) decreases renal mass in the subject. In some embodiments, the renal mass is reduced in the treated subject by at least about 1-10%, 10-20%, 20-30%, 40-50%. In some embodiments, the renal mass is reduced in the treated subject by at least 1-10%, 10-20%, 20-30%, 40-50%. In one embodiment, the renal mass in the treated subject is reduced by at least about 10%. In one embodiment, the renal mass in the treated subject is reduced by at least about 20%. In one embodiment, the renal mass in the treated subject is reduced by at least about 30%. In one embodiment, the renal mass in the treated subject is reduced by at least about 40%. In one embodiment, the renal mass in the treated subject is reduced by at least about 50%. In some embodiments, glomeruli number is increased in the kidney of the treated subject. For example, glomeruli number may be increased by about 1-10%, 10-20%, 20-30%, 40-50%, 50-60%, 60-70%, 70-80%, 80-90%, or 90-100% in the treated subject. For example, glomeruli number may be increased by 1-10%, 10-20%, 20-30%, 40-50%, 50-60%, 60-70%, 70-80%, 80-90%, or 90-100% in the treated subject.

The mTOR signaling pathway regulates many transcriptional and post-transcriptional modification events (e.g., FIG. 1). Diseases characterized by increased mTOR activity can lead to deregulation of the mTOR pathway. For example, proteins or messengers such as PIP2, PIP3, PDK, Akt, PTEN, PRAS40, GSK-3β, p21, p27 may be present in abnormal amounts in affected subjects and can be identified by any assays known in the art (e.g., western blot of protein lysates or immunohistochemistry analysis of tissue samples). Additionally, the phosphorylation state of proteins downstream of mTOR signaling, such as BAD, FOXO, NF-KB, p21Cip1, p27Kip1, GSK3β, TSC2 and others, which can be identified by any assays known in the art, may be altered in affected subjects. In one embodiment, treatment with a compound of Formula (I) decreases the amount of phospho-Akt-pS473 and phospho-Akt-pT308, phospho-4E-BP1, and/or phosphor-S6-RP in a treated subject.

In another aspect, the invention provides for a method comprising (a) evaluating whether a subject is susceptible to PKD, wherein said evaluation comprises testing for (i) the presence of a biomarker correlated with PKD in said subject; and/or (ii) the presence of multiple kidney cysts, and (b) administering to the subject being tested for (a)(i) and/or (a)(ii) an effective amount of a pharmaceutical composition comprising a compound of Formula (I) (e.g. an mTOR inhibitor), wherein the compound (e.g. mTOR inhibitor) is a compound of Formula (I). In some embodiments, the biomarker is a mutated PKD1 or PKD2 gene or gene product. Exemplary biomarkers are described in U.S. Patent Application Publication No. US20100047785A1 and US20050100898A1, each of which is herein incorporated by reference in its entirety. Biomarkers may be identified by any assays known in the art (e.g. RT-PCR of RNA from blood or other tissue samples). In some embodiments, imaging analysis is used for the detecting the presence of multiple cysts. Examples of imaging analysis include, without limitation, ultrasound or magnetic resonance imaging.

In another aspect, the invention provides for a method of treating an autosomal polycystic disease in a subject in need thereof, the method comprising administering to the subject a therapeutically effective amount of a compound of Formula (I) (e.g. an mTOR inhibitor), wherein the compound (e.g. mTOR inhibitor) is a compound of Formula (I) (including embodiments), wherein said administration is prior to, concurrent with, or after administration of another treatment to the subject in need thereof. Autosomal polycystic disorders, e.g. PKD, may be characterized by abnormal proliferative, fluid secretory, matrix regulatory, and/or apoptotic activity. In one embodiment, the “another treatment” comprises a therapeutic agent which is antiproliferative, an inhibitor of fluid secretion, and/or an inhibitor of matrix degradation. Exemplary therapeutic agents include, without limitation, inhibitors of cAMP (e.g., vasopressin V2R antagonists or Somatostatin), CA²⁺ signaling (e.g., triptolide), cyclin-dependent kinase (cdk) (e.g., Roscovitine), EGF receptor (e.g., tyrosine kinase inhibitors), metalloproteases (e.g., Batimastat), caspases (e.g., caspase inhibitors), and peroxisome-proliferator-activated receptor-γ (PPAR-γ) (e.g., Pioglitazone) activity. In one embodiment, the “another treatment” comprises treating symptoms of abnormal cyst growth/formation. Exemplary methods for treating symptoms of abnormal cyst growth/formation include, without limitation, percutaneous aspiration alone or with sclerotherapy, surgical decortications, laparoscopic surgery, and/or kidney transplantation. In one embodiment, the method comprises administering to the subject a therapeutically effective amount of a compound of Formula (I) (e.g. an mTOR inhibitor), wherein the compound (e.g. mTOR inhibitor) is a compound of Formula (I) (including embodiments), wherein said administration is prior to, concurrent with, or after administration of a therapeutically effective amount of an agent which is an inhibitor of proliferation, fluid secretion, and/or an matrix degradation. In one embodiment, the method comprises administering to the subject a therapeutically effective amount of a compound of Formula (I) (e.g. an mTOR inhibitor), wherein the compound (e.g. mTOR inhibitor) is a compound of Formula (I) (including embodiments), wherein said administration is prior to, concurrent with, or after administration of a therapeutically effective amount of an agent which is a inhibitor of cAMP, CA²⁺ signaling, cyclin-dependent kinase (cdk), EGF receptor, metalloprotease, caspase, and/or peroxisome-proliferator-activated receptor-γ (PPAR-γ) activity. In one embodiment, the method comprises administering to the subject a therapeutically effective amount of a compound of Formula (I) (e.g. an mTOR inhibitor), wherein the compound (e.g. mTOR inhibitor) is a compound of Formula (I) (including embodiments), wherein said administration is prior to, concurrent with, or after administering to the subject percutaneous aspiration alone or with sclerotherapy, surgical decortications, laparoscopic surgery, or kidney transplantation.

In some embodiments, a method of treating a condition caused by aberrant ion transport across epithelial cells in a patient in need thereof is provided. The method includes administering to the patient a therapeutically effective amount of a biologically active agent (e.g. compound of Formula (I) including embodiments and aspects) that selectively inhibits mTOR activity, wherein the compound (e.g. mTOR inhibitor) is a compound of Formula (I) (including embodiments).

A condition caused by aberrant ion (e.g., sodium ion, proton, lithium ion, potassium ion) transport across epithelial cells is a condition that would not occur but for the presence of aberrant ion transport across at least some epithelial cells in the patient. The epithelial cells typically form at least part of glands, connective tissue (e.g., the outer layer of connective tissues) and/or tissues lining the cavities of surfaces of structures (e.g., organs) throughout the body. In some embodiments, the epithelial cells are renal, liver, or pancreas epithelial cells. In some embodiments, the condition caused by aberrant ion transport across epithelial cells is a condition caused by aberrant ion transport across kidney epithelial cells, such as kidney collecting duct cells. The condition caused by aberrant ion transport across epithelial cells may also be a disease caused by aberrant sodium ion transport across epithelial cells, such as ENaC-dependent Na⁺ transport in renal epithelial cells. The collecting duct is the major site for cyst generation in the autosomal dominant and autosomal recessive forms of human polycystic kidney disease (PKD). Cysts may form due to abnormal cellular proliferation, and abnormal ion and fluid transport, which fill the cysts. Therefore, in some embodiments, the condition caused by aberrant ion transport across epithelial cells is PKD, a disease of collecting duct cell proliferation kidney (e.g., cyst formation), a blood pressure disease, a kidney electrolyte disorders, hypertension, congestive heart failure, nephrotic syndrome and/or cirrhosis of the liver.

In other embodiments, the biologically active agent is capable of inhibiting cyst progression in animal models of PKD to a greater degree than rapamycin. In other embodiments, the biologically active agent inhibits (e.g., decreases) ion transport processes in kidney tubule cells relative to the amount of ion transport in the absence of the biologically active agent. In other embodiments, the biologically active agent is excreted in the kidney. In other embodiments, the biologically active agent inhibits (e.g., decreases) phosphorylation and/or activation of SGK1, a key mediator of hormone-regulated Na⁺ transport, relative to the amount of phosphorylation and/or activation of SGK1 in the absence of the biologically active agent.

C. Pharmaceutical Compositions and Administration

The invention provides, in one aspect, a treatment utilizing a compound of Formula (I) (e.g. an mTOR inhibitor). Administration of the compounds of the present invention can be effected by any method that enables delivery of the compounds to the site of action. An effective amount of an inhibitor of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, administration of a compound of Formula (I) (e.g. an mTOR inhibitor) of the invention can be effected in one dose, continuously or intermittently throughout the course of treatment. Methods of determining the most effective means and dosage of administration are well known to those of skill in the art and will vary with the composition used for therapy, the purpose of the therapy, the target cell or tissue being treated, and the subject being treated. Single or multiple administrations can be carried out with the dose level and pattern being selected by the treating physician.

The amount of inhibitor or compound administered will be dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. Effective dosage may be in the range of about 0.001-0.01, 0.01-0.05, 0.05-0.1, 0.1-0.5, 0.5-1.0, 1-10, 10-50, 50-100, 100 or more mg/kg body weight/day. Effective dosage may be in the range of 0.001-0.01, 0.01-0.05, 0.05-0.1, 0.1-0.5, 0.5-1.0, 1-10, 10-50, 50-100, 100 or more mg/kg body weight/day. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a treatment of the invention is administered in multiple doses. Dosing may be about once, twice, three times, four times, five times, six times, or more than six times per day. Dosing may be about once a month, once every two weeks, once a week, or once every other day. In yet another embodiment the administration continues for more than about 6, 10, 14, 28 days, two months, six months, or one year. Dosing may be once, twice, three times, four times, five times, six times, or more than six times per day. Dosing may be once a month, once every two weeks, once a week, or once every other day. In yet another embodiment the administration continues for more than 6, 10, 14, 28 days, two months, six months, or one year. In some cases, continuous dosing is achieved and maintained as long as necessary.

Administration of the treatments of the invention may continue as long as necessary. In some embodiments, an agent of the invention is administered for more than 1, 2, 3, 4, 5, 6, 7, 14, or 28 days. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

When a treatment of the invention is administered as a composition that comprises one or more compounds, and one compound has a shorter half-life than another compound, the unit dose forms may be adjusted accordingly.

In some embodiments, treatments of the invention are tested to estimate pharmacokinetic properties and expected side effect profile. Various assays are known in the art for this purpose. For example, oral availability can be estimated during early stages of drug development by performing a Caco-2 permeability assay. Further, oral pharmacokinetics in humans can be approximated by extrapolating from the results of assays in mice, rats or monkey. In some embodiments, compounds of the invention show good oral availability across multiple species of organisms.

Other assays examine the effect of a compound of Formula (I) (e.g. an mTOR inhibitor) on liver function and metabolism. Cytochrome P450 (CYP) proteins are the main enzyme involved in metabolizing drugs administered to mammalian organisms. As such, undesired interaction of a drug candidate can be a significant source of adverse drug interactions. Generally, it is desirable for a drug to not interact with CYP isozymes such as CYP1A2, CYP2C9, CYP2C19, CYP2D6, or CYP3A4. In some embodiments, an inhibitor of the invention exhibits an IC50 of greater than 10 μM for CYP1A2, CYP2C9, CYP2C19, CYP2D6, or CYP3A4. Additionally, liver microsome and hepatocyte metabolism assays using human preparations can be used to estimate the in-vitro half life of a drug candidate.

Cardiac toxicity is also an important consideration in evaluating compounds. For example, hERG is the gene coding for the Kv11.1 potassium ion channel, a protein is involved in mediating repolarizing current in the cardiac action potential in the heart. Inhibition of the hERG gene product by a drug candidate can lead to an increase in the risk of sudden death and is therefore an undesirable property. In some embodiments, an inhibitor of the invention exhibits less than 10% hERG inhibition when administered at a suitable concentration.

Mutagenicity of compounds can be assayed via an Ames test or a modified Ames test using e.g., the liver S9 system. In some embodiments, compounds show negative activity in such a test.

Other undesired interactions of an inhibitor can also be ascertained via a receptor panel screen. In some embodiments, no significant interactions are detected for combination treatments of the invention. The subject pharmaceutical compositions can be formulated to provide a therapeutically effective amount of a combination of therapeutic agents of the present invention, or pharmaceutically acceptable salts, esters, prodrugs, solvates, hydrates or derivatives thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered as an mTOR inhibitor, or in further combination with one or more other agents, which are also typically administered in the form of pharmaceutical compositions. Where desired, the subject inhibitor and other agent(s) may be mixed into a preparation or both components may be formulated into separate preparations to use them in combination separately or at the same time.

In some embodiments, the concentration of a compound provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of a compound of the present invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of a compound of the present invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the concentration of a compound of the present invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of a compound of the present invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of the present invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of a compound of the present invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The treatments according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the present invention typically contains an active ingredient (e.g., a compound as described herein, a compound of Formula (I) (including embodiments), an inhibitor of the present invention or a pharmaceutically acceptable salt and/or coordination complex thereof), and one or more pharmaceutically acceptable excipients, carriers, including but not limited inert solid diluents and fillers, diluents, sterile aqueous solution and various organic solvents, permeation enhancers, solubilizers and adjuvants.

Described below are non-limiting exemplary pharmaceutical compositions and methods for preparing the same.

Pharmaceutical Compositions for Oral Administration.

In some embodiments, the invention provides a pharmaceutical composition for oral administration containing at least one therapeutic agent, and a pharmaceutical excipient suitable for oral administration.

In some embodiments, the invention provides a solid pharmaceutical composition for oral administration containing: (i) a compound of Formula (I) (e.g. an mTOR inhibitor) (e.g. compound of Formula (I) including embodiments); and (ii) a pharmaceutical excipient suitable for oral administration. In some embodiments, the composition further contains: (iii) a third agent or even a fourth agent. In some embodiments, each compound or agent is present in a therapeutically effective amount. In other embodiments, one or more compounds or agents is present in a sub-therapeutic amount, and the compounds or agents act synergystically to provide a therapeutically effective pharmaceutical composition.

In one embodiment, the present invention provides an oral dosage form comprising 100 mg to 1.5 g of an inhibitor of the invention. The oral dosage form can be a tablet, formulated in form of liquid, in immediate or sustained release format.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, including liquid dosage forms (e.g., a suspension or slurry), and oral solid dosage forms (e.g., a tablet or bulk powder). As used herein the term “tablet” refers generally to tablets, caplets, capsules, including soft gelatin capsules, and lozenges. Oral dosage forms may be formulated as tablets, pills, dragees, capsules, emulsions, lipophilic and hydrophilic suspensions, liquids, gels, syrups, slurries, suspensions and the like, for oral ingestion by an individual or a patient to be treated. Such dosage forms can be prepared by any of the methods of pharmacy, but all methods include the step of bringing the active ingredient into association with the carrier, which constitutes one or more necessary ingredients. In one embodiment, the inhibitor of the invention is contained in capsules. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. Optionally, the inventive composition for oral use can be obtained by mixing the inhibitor with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

Lubricants can be also be used in conjunction with tissue barriers which include, but are not limited to, polysaccharides, polyglycans, seprafilm, interceed and hyaluronic acid.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methylpyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical Compositions for Injection.

In some embodiments, the invention provides a pharmaceutical composition for injection containing at least one compound of the present invention and a pharmaceutical excipient suitable for injection. For example a pharmaceutical composition for injection is provided comprising a compound of Formula (I) (e.g. an mTOR inhibitor) (including embodiments). Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g., Transdermal) Delivery.

In some embodiments, the invention provides a pharmaceutical composition for transdermal delivery containing at least one compound of the present invention and a pharmaceutical excipient suitable for transdermal delivery. For example a pharmaceutical composition for topical delivery is provided comprising a compound of Formula (I) (e.g. an mTOR inhibitor) (including embodiments).

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of an inhibitor of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. For example a pharmaceutical composition for topical delivery is provided comprising a compound of Formula (I) (e.g. an mTOR inhibitor) (including embodiments). Compositions of a compound of Formula (I) (e.g. an mTOR inhibitor) (including embodiments) may be formulated may further include a second therapeutic agent.

Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

Administration of each compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The compounds of the invention may be administered in dosages. It is known in the art that due to intersubject variability in compound pharmacokinetics, individualization of dosing regimen is necessary for optimal therapy. Dosing for an inhibitor of the invention may be found by routine experimentation in light of the instant disclosure.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and an inhibitor according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

The biologically active agents of the invention may be administered in dosages as described herein. It is known in the art that due to intersubject variability in biologically active agent pharmacokinetics, individualization of dosing regimen is necessary for optimal therapy. Dosing for a biologically active agent of the invention may be found by routine experimentation.

The invention also provides kits. The kits include an inhibitor or compounds of the present invention as described herein (e.g. a compound of Formula (I)(including embodiments), in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention (e.g. a compound of Formula (I)(including embodiments) and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

In some embodiments, the subject is a human in need of treatment for an autosomal polycystic disorder. Subjects that can be treated with treatments of the present invention, or pharmaceutically acceptable salt, ester, prodrug, solvate, hydrate or derivatives of the therapeutic agents, according to the methods of this invention include, for example, subjects that have been diagnosed as having PKD (e.g., autosomal dominant polycystic kidney disease or autosomal recessive polycystic kidney disease) or autosomal dominant polycystic liver disease.

The invention further provides methods of modulating mTOR kinase activity by contacting the kinase with an effective amount of a compound of Formula (I) (e.g. an mTOR inhibitor) ((including embodiments). Modulation can be inhibiting or activating kinase activity. In some embodiments, the invention provides methods of inhibiting kinase activity by contacting the kinase with an effective amount of a composition comprising a compound of Formula (I) (e.g. an mTOR inhibitor) (including embodiments). In some embodiments, the invention provides methods of inhibiting the kinase activity by contacting a cell, tissue, or organ that expresses the kinase of interest. In some embodiments, the invention provides methods of inhibiting kinase activity in subject including but not limited to rodents and mammal (e.g., human) by administering into the subject an effective amount of a composition comprising a compound of Formula (I) (e.g. an mTOR inhibitor) (including embodiments). In some embodiments, the percentage of inhibition exceeds 50%, 60%, 70%, 80%, or 90%.

### Embodiment 1

A method of treating polycystic kidney disease (PKD) in a subject in need thereof, comprising administering to the subject a therapeutically effective amount of a compound of Formula (I):

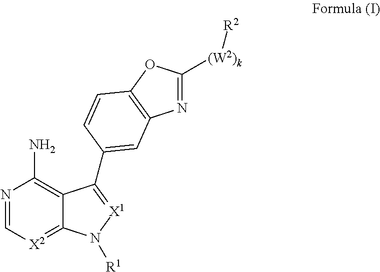

wherein:

### X¹ is N or C-E¹;

### X² is N or CH;

E¹ is —(W¹)ⱼ—R⁴;

### W¹ is —O—, —NR^{7A}—, —S(O)₀₋₂—, —C(O)—, —C(O)N(R^{7A})—, —N(R^{7A})C(O)—, or —N(R^{7A})C(O)N(R^{8A})—;

### W² is —O—, —NR⁷—, —S(O)₀₋₂—, —C(O)—, —C(O)N(R⁷)—, —N(R⁷)C(O)—, or —N(R⁷)C(O)N(R⁸)—;

j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹R³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂N(C₁₋₁₀alkyl)(C₁₋₁₀alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C(═O)NR³¹R³², —C(═O)NR³⁴R³⁵, —SO₂NR³⁴R³⁵, —SO₂NR³¹R³², —NR³¹R³², or —NR³⁴R³⁵.

### Embodiment 2

A method of treating a polycystic disease in a subject in need thereof, comprising administering to the subject a therapeutically effective amount of a compound of Formula (I):

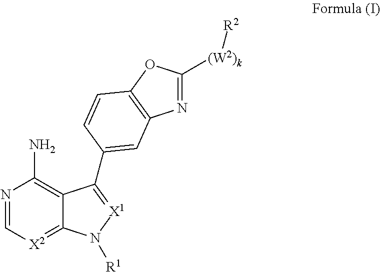

wherein:

### X¹ is N or C-E¹;

### X² is N or CH;

E¹ is —(W¹)ⱼ—R⁴;
W¹ is —O—, —NR^{7A}—, —S(O)₀₋₂, CO—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A}), —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is —H, -aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R¹ is unsubstituted or substituted with one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R² is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR OR³², —O-aryl or —SC(═O)NR³¹R³²;
R³ and R⁴ are independently hydrogen, halogen, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹R³², —SC(═O)NR³¹R³², aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein each R³ and R⁴ is independently unsubstituted or substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³²;
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C_{—10}alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or C₁₋₁₀alkyl, C₂₋₁₀alkenyl, C₂₋₁₀alkynyl, C₃₋₈cycloalkyl, heteroalkyl, aryl, heteroaryl, or heterocyclyl moiety, wherein each of said moieties is unsubstituted or is substituted with one or more C₁₋₁₀alkyl, C₂₋₁₀alkenyl, C₂₋₁₀alkynyl, C₃₋₁₀cycloalkyl, heteroalkyl, aryl, heteroaryl, or heterocyclyl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R⁷, R^{7A}, R⁸, and R^{8A} is independently hydrogen, C₁₋₁₀alkyl, C₂₋₁₀alkenyl, C₂₋₁₀alkynyl, aryl, heteroalkyl, heteroaryl, heterocyclyl or C₃₋₁₀cycloalkyl, each of which except for hydrogen is unsubstituted or is substituted by one or more independent R⁶ substituents; and
R⁶ is independently halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³²; or C₁₋₁₀alkyl, C₂₋₁₀alkenyl, C₂₋₁₀alkynyl, C₃₋₈cycloalkyl, heteroalkyl, aryl, heteroaryl, heterocyclyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl,
wherein each R⁶ is independently unsubstituted or substituted with one or more independent halo, oxo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

### Embodiment 3

The method of embodiment 2, wherein said polycystic disease is polycystic kidney disease.

### Embodiment 4

A method of treating a polycystic disease in a subject in need thereof, comprising administering to the subject a therapeutically effective amount of a compound of Formula (I):

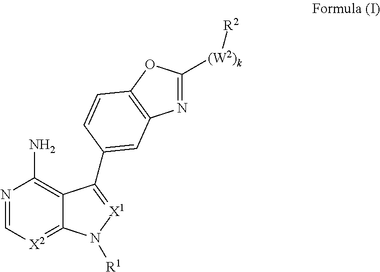

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is hydrogen, R³-substituted or unsubstituted C₁₋₁₀alkyl, R³-substituted or unsubstituted C₂₋₁₀alkenyl, R³-substituted or unsubstituted C₂₋₁₀alkynyl, R³-substituted or unsubstituted C₃₋₈cycloalkyl, R³-substituted or unsubstituted C₃₋₈cycloalkenyl, R³-substituted or unsubstituted C₃₋₈cycloalkynyl, R³-substituted or unsubstituted heteroalkyl, R³-substituted or unsubstituted heteroalkenyl, R³-substituted or unsubstituted heteroalkynyl, R³-substituted or unsubstituted heterocyclyl, R³-substituted or unsubstituted aryl, R³-substituted or unsubstituted heteroaryl; wherein each R³-substituted R¹ is independently substituted with one or more R³
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R² is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).
R³ and R⁴ are independently is hydrogen, oxo, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —C(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R³ or R⁴ is independently substituted with one or more independent halogen, —OH, oxo, —R³¹, —CF₃, —OCF₃, —OR³¹, —NR³¹R³², NR³⁴R³⁵, —C(O)R³¹, —CO₂R³¹, —C(═O)NR³¹R³², C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂N(C₁₋₁₀alkyl)(C₁₋₁₀alkyl), or —SO₂NH(C₁₋₁₀alkyl);
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C_{—10}alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₀N(C₁₋₁₀alkyl)(C₁₋₁₀alkyl), —SO₂NH(C₁₋₁₀alkyl), —COOH, or —SO₂NR³⁴R³⁵;
each R⁷, R^{7A}, R⁸, and R^{8A} is independently hydrogen, R⁶-substituted or unsubstituted C₁₋₁₀alkyl, R⁶-substituted or unsubstituted C₂₋₁₀alkenyl, R⁶-substituted or unsubstituted C₂₋₁₀alkynyl, R⁶-substituted or unsubstituted C₃₋₈cycloalkyl, R⁶-substituted or unsubstituted C₃₋₈cycloalkenyl, R⁶-substituted or unsubstituted C₃₋₈cycloalkynyl, R⁶-substituted or unsubstituted heteroalkyl, R⁶-substituted or unsubstituted heteroalkenyl, R⁶-substituted or unsubstituted heteroalkynyl, R⁶-substituted or unsubstituted heterocyclyl, R⁶-substituted or unsubstituted aryl, R⁶-substituted or unsubstituted heteroaryl; wherein each R⁶-substituted R⁷, R^{7A}, R⁸ and R^{8A} is independently substituted with one or more R⁶; and
R⁶ is independently halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R⁶ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).

### Embodiment 5

The method of embodiment 1, wherein the compound selectively inhibits both mTORC1 and mTORC2 activity.

### Embodiment 6

The method of embodiment 5, wherein the compound selectively inhibits both mTORC1 and mTORC2 activity relative to one or more type I phosphatidylinositol 3-kinases (PI3-kinase) as ascertained in a cell-based assay or an in vitro kinase assay, wherein the one or more type I PI3-kinase is selected from the group consisting of PI3-kinase α, PI3-kinase β, PI3-kinase γ, and PI3-kinase δ.

### Embodiment 7

The method of embodiment 1, wherein the subject is a mammal.

### Embodiment 8

The method of embodiment 1, wherein the compound inhibits mTOR activity with an IC₅₀ value of about 100 nM or less as ascertained in an in vitro kinase assay.

### Embodiment 9

The method of embodiment 1, wherein the compound inhibits mTOR activity with an IC₅₀ value of about 10 nM or less as ascertained in an in vitro kinase assay.

### Embodiment 10

The method of embodiment 1, wherein said administration of the mTOR inhibitor decreases kidney size, decreases cyst volume, and/or increases glomeruli number in the subject.

### Embodiment 11

The method of embodiment 1, wherein the compound is administered parenterally, orally, intraperitoneally, intravenously, intraarterially, transdermally, intramuscularly, liposomally, via local delivery by catheter or stent, subcutaneously, intraadiposally, or intrathecally.

### Embodiment 12

The method of embodiment 1, wherein said treatment reduces kidney mass in the subject by at least 10%.

### Embodiment 13

The method of embodiment 1, wherein said treatment reduces kidney mass in the subject by at least 50%.

### Embodiment 14

The method of embodiment 1, wherein said treatment reduces normalized kidney mass in the subject by at least 10%.

### Embodiment 15

The method of embodiment 1, wherein said treatment reduces normalized kidney mass in the subject by at least 30%.

### Embodiment 16

The method of embodiment 1, wherein said administration of the compound is prior to, concurrent with, or after administration of another treatment to the subject.

### Embodiment 17

A method of inhibiting cyst formation in a subject at risk for developing PKD, comprising contacting cyst cells with a compound of Formula (I) in an amount sufficient to inhibit growth of cyst cells:

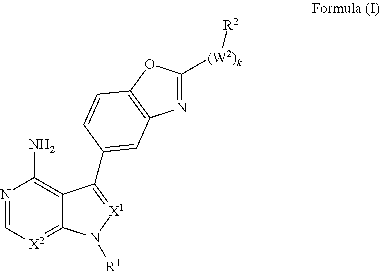

wherein:

### X¹ is N or C-E¹;

### X² is N or CH;

E¹ is —(W¹)ⱼ—R⁴;

### W¹ is —O—, —NR^{7A}—, —S(O)₀₋₂—, —C(O)—, —C(O)N(R^{7A})—, —N(R^{7A})C(O), or —N(R^{7A})C(O)N(R^{A})—;

### W² is —O—, —NR⁷—, —S(O)₀₋₂—, —C(O)—, —C(O)N(R⁷)—, —N(R⁷)C(O)—, or —N(R⁷)C(O)N(R⁸)—;

j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂N(C₁₋₁₀alkyl)(C_{—10}alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

### Embodiment 18

The method of embodiment 17, further comprising reducing cyst formation in an organ other than kidney.

### Embodiment 19

A method comprising:

(a) evaluating whether a subject is susceptible to PKD, wherein said evaluation comprises testing for (i) the presence of a biomarker correlated with PKD in said subject; and/or (ii) the presence of multiple kidney cysts; and
(b) administering to the subject being tested for (a)(i) and/or (a)(ii) a pharmaceutical composition comprising an effective amount of a compound of Formula (I):

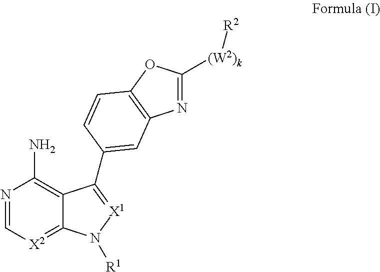

wherein:

### X¹ is N or C-E¹;

### X² is N or CH;

E¹ is —(W¹)ⱼ—R⁴;

### W¹ is —O—, —NR^{7A}—, —S(O)₀₋₂—, —C(O)—, —C(O)N(R^{7A})—, N(R^{7A})C(O)—, or —N(R^{7A})C(O)N(R^{8A})—;

### W² is —O—, —NR⁷—, —S(O)₀₋₂—, —C(O)—, —C(O)N(R⁷)—, —N(R⁷)C(O)—, or —N(R⁷)C(O)N(R⁸)—;

j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³¹R³², —C(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂N(C₁₋₁₀alkyl)(C_{—10}alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, C₂₋₁₀alkynyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂ C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C(═O)NR³¹R³², —C(═O)NR³⁴R³⁵, —SO₂NR³⁴R³⁵, —SO₂NR³¹R³², —NR³¹R³², or —NR³⁴R³⁵.

### Embodiment 20

The method of embodiment 19, wherein the biomarker is a mutated PKD-1 or PKD-2 gene, or a respective gene product.

### Embodiment 21

The method of any one of embodiments 2-4, wherein the compound selectively inhibits both mTORC1 and mTORC2 activity.

### Embodiment 22

The method of embodiment 21, wherein the compound selectively inhibits both mTORC1 and mTORC2 activity relative to one or more type I phosphatidylinositol 3-kinases (PI3-kinase) as ascertained in a cell-based assay or an in vitro kinase assay, wherein the one or more type I PI3-kinase is selected from the group consisting of PI3-kinase α, PI3-kinase β, PI3-kinase γ, and PI3-kinase δ.

### Embodiment 23

The method of any one of embodiments 2-4, wherein the subject is a mammal.

### Embodiment 24

The method of any one of embodiments 2-4, wherein the compound inhibits mTOR activity with an IC₅₀ value of about 100 nM or less as ascertained in an in vitro kinase assay.

### Embodiment 25

The method of any one of embodiments 2-4, wherein the compound inhibits mTOR activity with an IC₅₀ value of about 10 nM or less as ascertained in an in vitro kinase assay.

### Embodiment 26

The method of any one of embodiments 2-4, wherein said administration of the compound decreases kidney size, decreases cyst volume, and/or increases glomeruli number in the subject.

### Embodiment 27

The method of any one of embodiments 2-4, wherein the compound is administered parenterally, orally, intraperitoneally, intravenously, intraarterially, transdermally, intramuscularly, liposomally, via local delivery by catheter or stent, subcutaneously, intraadiposally, or intrathecally.

### Embodiment 28

The method of any one of embodiments 2-4, wherein said treatment reduces kidney mass in the subject by at least 10%.

### Embodiment 29

The method of any one of embodiments 2-4, wherein said treatment reduces kidney mass in the subject by at least 50%.

### Embodiment 30

The method of any one of embodiments 2-4, wherein said treatment reduces normalized kidney mass in the subject by at least 10%.

### Embodiment 31

The method of any one of embodiments 2-4, wherein said treatment reduces normalized kidney mass in the subject by at least 30%.

### Embodiment 32

The method of any one of embodiments 2-4, wherein said administration of the compound is prior to, concurrent with, or after administration of another treatment to the subject.

### Embodiment 33

A method of inhibiting cyst formation in a subject at risk for developing PKD, comprising contacting cyst cells with compound of Formula (I) in an amount sufficient to inhibit growth of cyst cells:

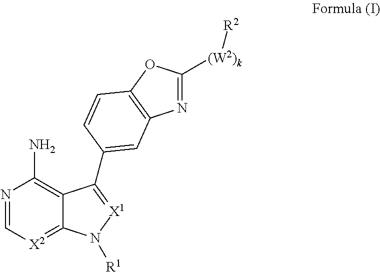

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is —H, -aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R¹ is unsubstituted or substituted with one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R² is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR OR³², —O-aryl or —SC(═O)NR³¹R³²;
R³ and R⁴ are independently hydrogen, halogen, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹R³², —SC(═O)NR³¹R³², aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein each R³ and R⁴ is independently unsubstituted or substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³²;
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C_{—10}alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or C₁₋₁₀alkyl, C₂₋₁₀alkenyl, C₂₋₁₀alkynyl, C₃₋₈cycloalkyl, heteroalkyl, aryl, heteroaryl, or heterocyclyl moiety, wherein each of said moieties is unsubstituted or is substituted with one or more C₁₋₁₀alkyl, C₂₋₁₀alkenyl, C₂₋₁₀alkynyl, C₃₋₁₀cycloalkyl, heteroalkyl, aryl, heteroaryl, or heterocyclyl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C_{—10}alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C_{—10}alkyl), —SO₂NH(C₁₋₁₀alkyl), —COOH, or —SO₂NR³⁴R³⁵;
each R⁷, R^{7A}, R⁸, and R^{8A} is independently hydrogen, C₁₋₁₀alkyl, C₂₋₁₀alkenyl, C₂₋₁₀alkynyl, aryl, heteroalkyl, heteroaryl, heterocyclyl or C₃₋₁₀cycloalkyl, each of which except for hydrogen is unsubstituted or is substituted by one or more independent R⁶ substituents; and
R⁶ is independently halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³²; or C₁₋₁₀alkyl, C₂₋₁₀alkenyl, C₂₋₁₀alkynyl, C₃₋₈cycloalkyl, heteroalkyl, aryl, heteroaryl, heterocyclyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl,
wherein each R⁶ is independently unsubstituted or substituted with one or more independent halo, oxo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

### Embodiment 34

A method of inhibiting cyst formation in a subject at risk for developing PKD, comprising contacting cyst cells with compound of Formula (I) in an amount sufficient to inhibit growth of cyst cells:

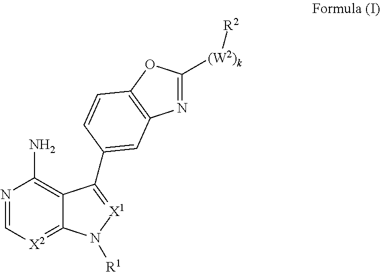

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is hydrogen, R³-substituted or unsubstituted C₁₋₁₀alkyl, R³-substituted or unsubstituted C₂₋₁₀alkenyl, R³-substituted or unsubstituted C₂₋₁₀alkynyl, R³-substituted or unsubstituted C₃₋₈cycloalkyl, R³-substituted or unsubstituted C₃₋₈cycloalkenyl, R³-substituted or unsubstituted C₃₋₈cycloalkynyl, R³-substituted or unsubstituted heteroalkyl, R³-substituted or unsubstituted heteroalkenyl, R³-substituted or unsubstituted heteroalkynyl, R³-substituted or unsubstituted heterocyclyl, R³-substituted or unsubstituted aryl, R³-substituted or unsubstituted heteroaryl; wherein each R³-substituted R¹ is independently substituted with one or more R³
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R² is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).
R³ and R⁴ are independently is hydrogen, oxo, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R³ or R⁴ is independently substituted with one or more independent halogen, —OH, oxo, —R³¹, —CF₃, —OCF₃, —OR³¹, —NR³¹R³², NR³⁴R³⁵, —C(O)R³¹, —CO₂R³¹, —C(═O)NR³¹R³², C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl);
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R⁷, R^{7A}, R⁸, and R^{8A} is independently hydrogen, R⁶-substituted or unsubstituted C₁₋₁₀alkyl, R⁶-substituted or unsubstituted C₂₋₁₀alkenyl, R⁶-substituted or unsubstituted C₂₋₁₀alkynyl, R⁶-substituted or unsubstituted C₃₋₈cycloalkyl, R⁶-substituted or unsubstituted C₃₋₈cycloalkenyl, R⁶-substituted or unsubstituted C₃₋₈cycloalkynyl, R⁶-substituted or unsubstituted heteroalkyl, R⁶-substituted or unsubstituted heteroalkenyl, R⁶-substituted or unsubstituted heteroalkynyl, R⁶-substituted or unsubstituted heterocyclyl, R⁶-substituted or unsubstituted aryl, R⁶-substituted or unsubstituted heteroaryl; wherein each R⁶-substituted R⁷, R^{7A}, R⁸ and R^{8A} is independently substituted with one or more R⁶; and
R⁶ is independently halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R⁶ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂N(C₁₋₁₀alkyl)(C₁₋₁₀alkyl), or —SO₂NH(C₁₋₁₀alkyl).

### Embodiment 35

The method of embodiment 33 or 34, further comprising reducing cyst formation in an organ other than kidney.

### Embodiment 36

A method comprising:
(a) evaluating whether a subject is susceptible to PKD, wherein said evaluation comprises testing for (i) the presence of a biomarker correlated with PKD in said subject; and/or (ii) the presence of multiple kidney cysts; and
(b) administering to the subject being tested for (a)(i) and/or (a)(ii) a pharmaceutical composition comprising an effective amount of a compound of Formula (I):

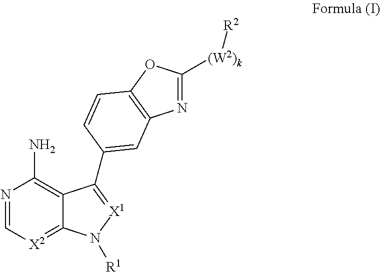

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is —H, -aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R¹ is unsubstituted or substituted with one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R² is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR OR³², —O-aryl or —SC(═O)NR³¹R³²;
R³ and R⁴ are independently hydrogen, halogen, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein each R³ and R⁴ is independently unsubstituted or substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³²;
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or C₁₋₁₀alkyl, C₂₋₁₀alkenyl, C₂₋₁₀alkynyl, C₃₋₈cycloalkyl, heteroalkyl, aryl, heteroaryl, or heterocyclyl moiety, wherein each of said moieties is unsubstituted or is substituted with one or more C₁₋₁₀alkyl, C₂₋₁₀alkenyl, C₂₋₁₀alkynyl, C₃₋₁₀cycloalkyl, heteroalkyl, aryl, heteroaryl, or heterocyclyl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R⁷, R^{7A}, R⁸, and R^{8A} is independently hydrogen, C₁₋₁₀alkyl, C₂₋₁₀alkenyl, C₂₋₁₀alkynyl, aryl, heteroalkyl, heteroaryl, heterocyclyl or C₃₋₁₀cycloalkyl, each of which except for hydrogen is unsubstituted or is substituted by one or more independent R⁶ substituents; and
R⁶ is independently halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³²; or C₁₋₁₀alkyl, C₂₋₁₀alkenyl, C₂₋₁₀alkynyl, C₃₋₈cycloalkyl, heteroalkyl, aryl, heteroaryl, heterocyclyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl,
wherein each R⁶ is independently unsubstituted or substituted with one or more independent halo, oxo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

### Embodiment 37

A method comprising:
(a) evaluating whether a subject is susceptible to PKD, wherein said evaluation comprises testing for (i) the presence of a biomarker correlated with PKD in said subject; and/or (ii) the presence of multiple kidney cysts; and
(b) administering to the subject being tested for (a)(i) and/or (a)(ii) a pharmaceutical composition comprising an effective amount of a compound of Formula (I):

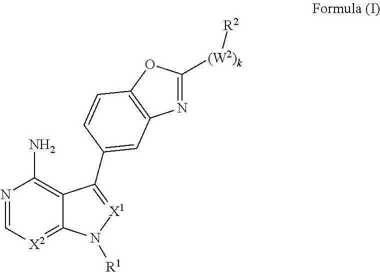

wherein:

### X¹ is N or C-E¹;

### X² is N or CH;

E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, N(R⁷)C(O), —N(R⁷)S(O)—, —N(R⁷)S(O)₂—, —C(O)O—, CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is hydrogen, R³-substituted or unsubstituted C₁₋₁₀alkyl, R³-substituted or unsubstituted C₂₋₁₀alkenyl, R³-substituted or unsubstituted C₂₋₁₀alkynyl, R³-substituted or unsubstituted C₃₋₈cycloalkyl, R³-substituted or unsubstituted C₃₋₈cycloalkenyl, R³-substituted or unsubstituted C₃₋₈cycloalkynyl, R³-substituted or unsubstituted heteroalkyl, R³-substituted or unsubstituted heteroalkenyl, R³-substituted or unsubstituted heteroalkynyl, R³-substituted or unsubstituted heterocyclyl, R³-substituted or unsubstituted aryl, R³-substituted or unsubstituted heteroaryl; wherein each R³-substituted R¹ is independently substituted with one or more R³
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, NR³¹C(═NR³²)NR³³R³², —NR³¹C(═NR³²)OR³³, —NR³¹C(═NR³²)SR³³, —OC(═O)OR³³, —OC(═O)NR³¹R³², OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R² is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹R³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).
R³ and R⁴ are independently is hydrogen, oxo, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R³ or R⁴ is independently substituted with one or more independent halogen, —OH, oxo, —R³¹, —CF₃, —OCF₃, —OR³¹, —NR³¹R³², NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl);
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C_{—10}alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R⁷, R^{7A}, R⁸, and R^{8A} is independently hydrogen, R⁶-substituted or unsubstituted C₁₋₁₀alkyl, R⁶-substituted or unsubstituted C₂₋₁₀alkenyl, R⁶-substituted or unsubstituted C₂₋₁₀alkynyl, R⁶-substituted or unsubstituted C₃₋₈cycloalkyl, R⁶-substituted or unsubstituted C₃₋₈cycloalkenyl, R⁶-substituted or unsubstituted C₃₋₈cycloalkynyl, R⁶-substituted or unsubstituted heteroalkyl, R⁶-substituted or unsubstituted heteroalkenyl, R⁶-substituted or unsubstituted heteroalkynyl, R⁶-substituted or unsubstituted heterocyclyl, R⁶-substituted or unsubstituted aryl, R⁶-substituted or unsubstituted heteroaryl; wherein each R⁶-substituted R⁷, R^{7A}, R⁸, and R^{8A} is independently substituted with one or more R⁶; and
R⁶ is independently halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R⁶ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).

Within each aspect and each embodiment above:

Each R⁴ in a compound may be independently different. Each R⁵ in a compound may be independently different. Each R⁶ in a compound may be independently different. Each R⁷ in a compound may be independently different. Each R^{7A} in a compound may be independently different. Each R⁸ in a compound may be independently different. Each R^{8A} in a compound may be independently different. Each R³¹ in a compound may be independently different. Each R³² in a compound may be independently different. Each R³³ in a compound may be independently different. Each R³⁴ in a compound may be independently different. Each R³⁵ in a compound may be independently different. For example, a compound comprising and R⁶ substituted R^{7A} and a R⁶ substituted R⁷ may have a particular R⁶ (e.g. C1alkyl) on R^{7A} and a different R⁶ on R⁷ (e.g. phenyl). Furthermore, each occurrence of a moiety such as C₁₋₁₀alkyl, which encompasses multiple groups may each be a different member of that group (e.g. one a methyl and another an ethyl). The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### EXAMPLES

### Example 1

### Biochemical Properties of Compound A

Purified mTOR kinase domain was incubated with inhibitors at 2- or 4-fold dilutions over a concentration range of 50-0.001 μM or with vehicle (0.1% DMSO) in the presence of 10 μM ATP, 2.5 μCi of γ-32P-ATP and substrate. Reactions were terminated by spotting onto nitrocellulose or phosphocellulose membranes, depending on the substrate; this membrane was then washed 5-6 times to remove unbound radioactivity and dried. Transferred radioactivity was quantified by phosphorimaging and IC₅₀ values were calculated by fitting the data to a sigmoidal dose-response using Prism software.

Compound A was shown to be a specific inhibitor of mTOR with an IC₅₀ of less than 100 nM. Additionally, Compound A showed selective inhibition of mTOR, being over 1000× more effective in inhibiting mTOR activity than that of PI3K (α, β, γ, and δ). Compound A was also shown to inhibit cellular proliferation with IC₅₀ of about 10-100 nM. Similarly, compounds B, C and D were shown to be specific inhibitors of mTOR with an IC₅₀ of less than 100 nM.

### Example 2

### Effects of Treatment with Compound A in Mouse Model of PKD

The collecting duct is the major site for cyst generation in autosomal dominant and autosomal recessive forms of human polycystic kidney disease (PKD). Cysts form due to abnormal cellular proliferation, in conjunction with abnormal ion and fluid transport, which fills the cysts. Both mTORC1 and mTORC2-dependent outputs are hyperphosphorylated in human ADPKD.

In order to study mechanisms of selective intervention for treating PKD, PKD(V/V) (“V/V”) mice were used. V/V mice are an animal model of PKD which are homozygous for a mutation in PKD 1 (the gene implicated in 85% of PKD). (Yu S, et al. Proc. Natl. Acad. Sci. U.S.A. 2007; 104: 18688-18693.) These mice develop severe PKD rapidly during the postnatal period. P11 V/V mice were treated with Compound A (0.5 mg/kg; “+”) or vehicle (“−”) p.o. Animals were sacrificed 2 hours later and kidney crude lysates were prepared and subjected to Western blot analysis.

Whole kidneys were isolated from mice and immediately flash frozen. Tissues were ground using a mortar and pestle under a blanket of liquid nitrogen followed by homogenization in lysis buffer. Supernatants were loaded in gels and proteins were separated via electrophoresis. Primary antibodies were used to assess the following proteins: p-Akt (S473), p-Akt (T308), total-Akt, p-4E-BP1, total-4E-BP1, p-S6 ribosomal protein, and total-S6 ribosomal protein.

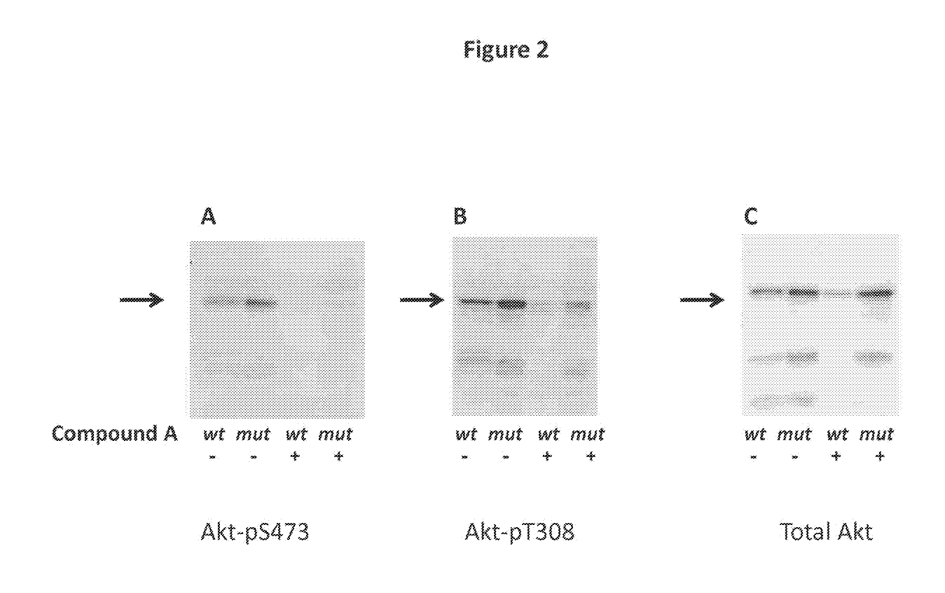

Lystates were examined for effects of Compound A on p-AKT. Akt-S473 and T308 phosphorylation were elevated in mutant mice, indicating hyperactivated mTOR activity in the kidneys of V/V mice. (FIG. 2A) S473 and T308 phosphorylation was moderately attenuated by Compound A. (FIG. 2B) Total Akt was increased in mutant mice, but is unaffected by Compound A. (FIG. 2C) These findings were consistent in several experiments.

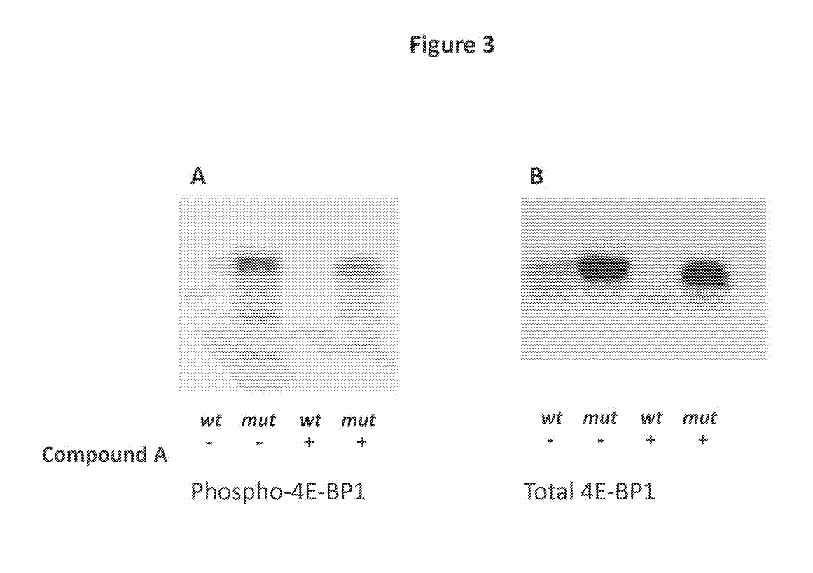

Western blots were stripped and restained with antibodies to examine for effects of Compound A on p-4EBP1 total 4E-BP1. p4E-BPI is markedly elevated in mutant mice, and markedly inhibited by Compound A. (FIG. 3A) Baseline phosphorylation and expression of 4E-BP1 in wt mice was low, but also markedly attenuated by Compound A. (FIG. 3B)

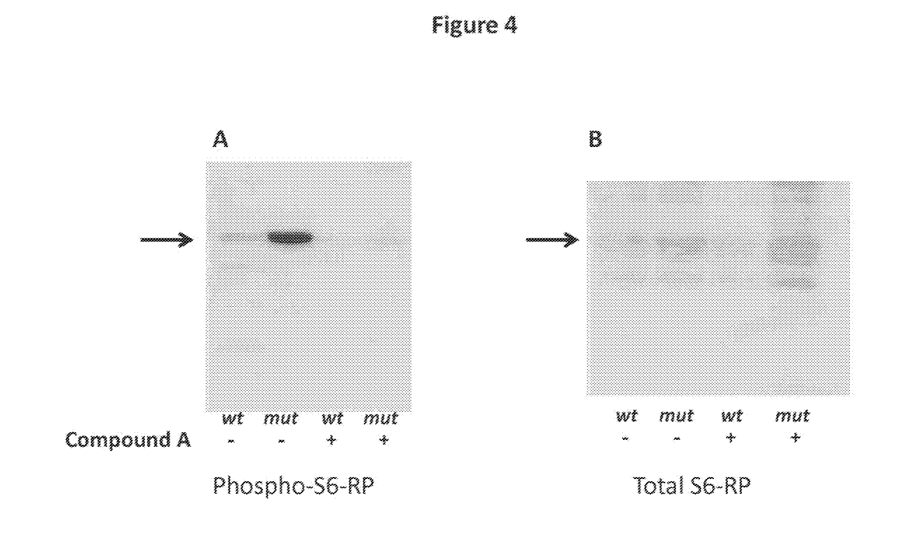

Western blots were stripped and restained with antibodies to examine for effects of Compound A on phospho-S6-RP and total S6-RP ribosomal protein. pS6-RP was markedly elevated in mutant mice, and markedly inhibited by Compound A. (FIG. 4A) Baseline phosphorylation of S6 was low in wt and mutant mice, but also markedly attenuated by Compound A. (FIG. 4A) S6-RP expression was relatively unaffected by mutation or Compound A. (FIG. 4B)

To examine Compound A effects on kidney size in V/V mice, V/V mice were treated from P5-P11 with either vehicle or Compound A. Compound A dosing was 0.25 mg/kg on P5/P6, then 0.25 mg/kg bid on P7/P8, then 0.5 mg/kg bid on P9-11. Animals were sacrificed 2 hours after fast dose; kidneys were weighed, and one kidney was subjected to western blot and one to sectioning for histology.

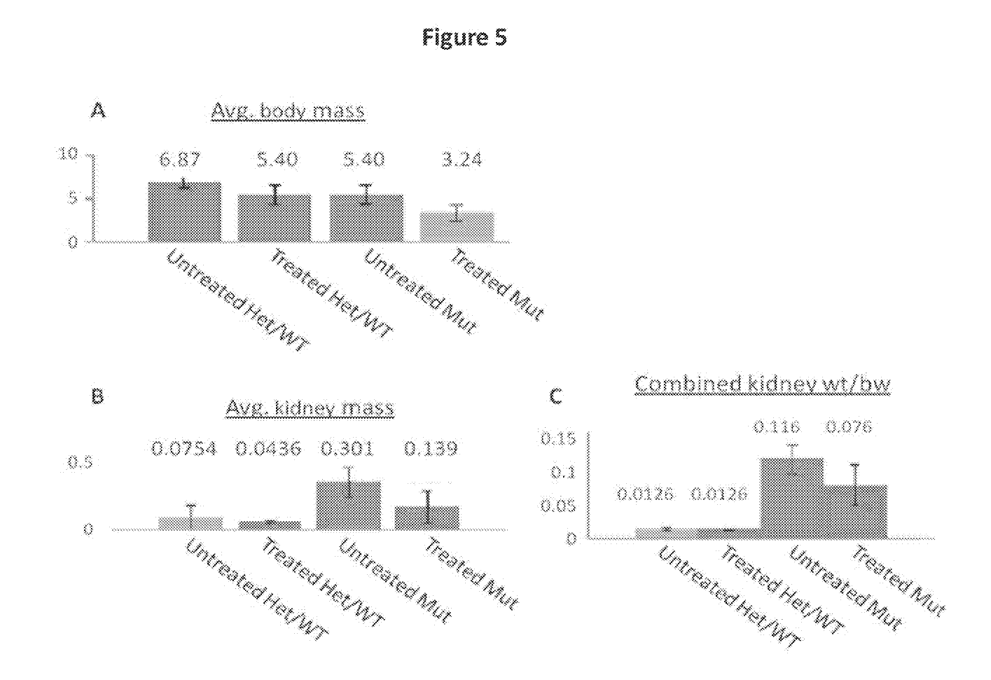

Body mass in mutant and in Het/WT mice was decreased by treatment with Compound A (p<0.05). (FIG. 5A) Average kidney mass was significantly lower in Compound A-treated as compared to vehicle treated mutants (p=0.007). (FIG. 5B) In contrast, kidney mass was not significantly changed by Compound A in Het/WT (p=0.22). Normalized kidney mass (combined kidney weight/body weight) was significantly lower in Compound A-treated compared with vehicle-treated mutants (p=0.01). (FIG. 5C) Compound A had no significant effect on normalized kidney mass in Het/WT mice (p=0.5).

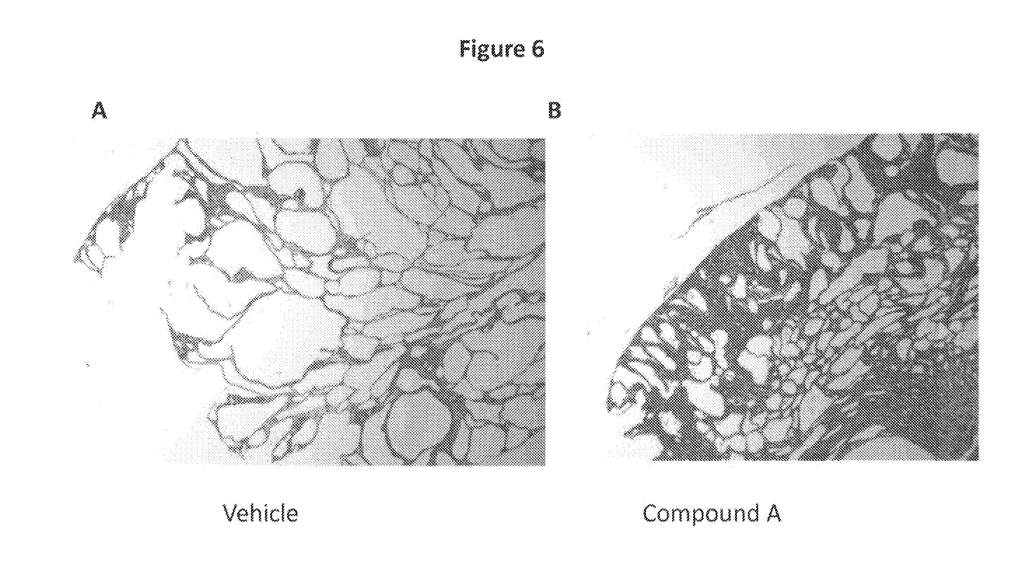

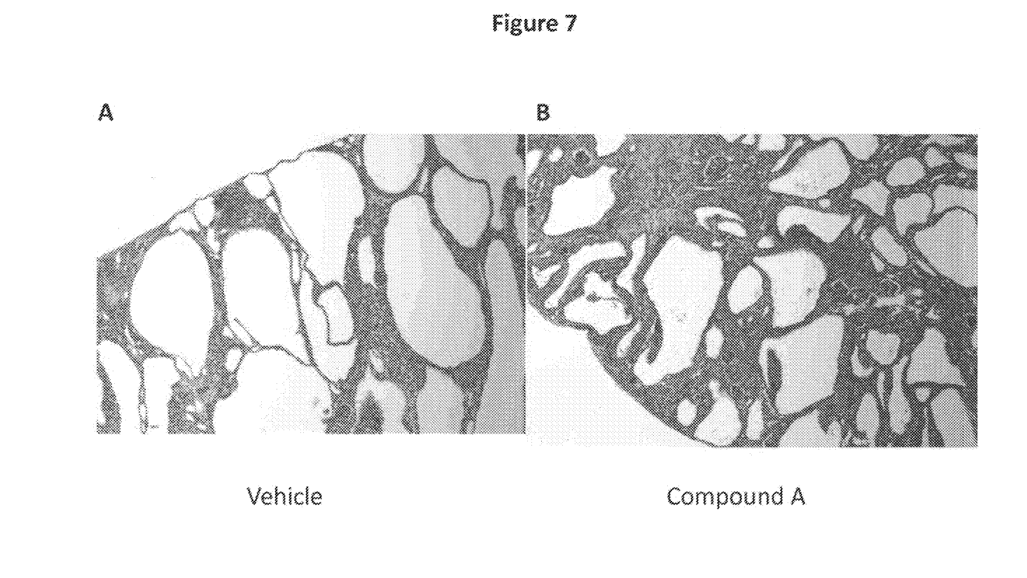

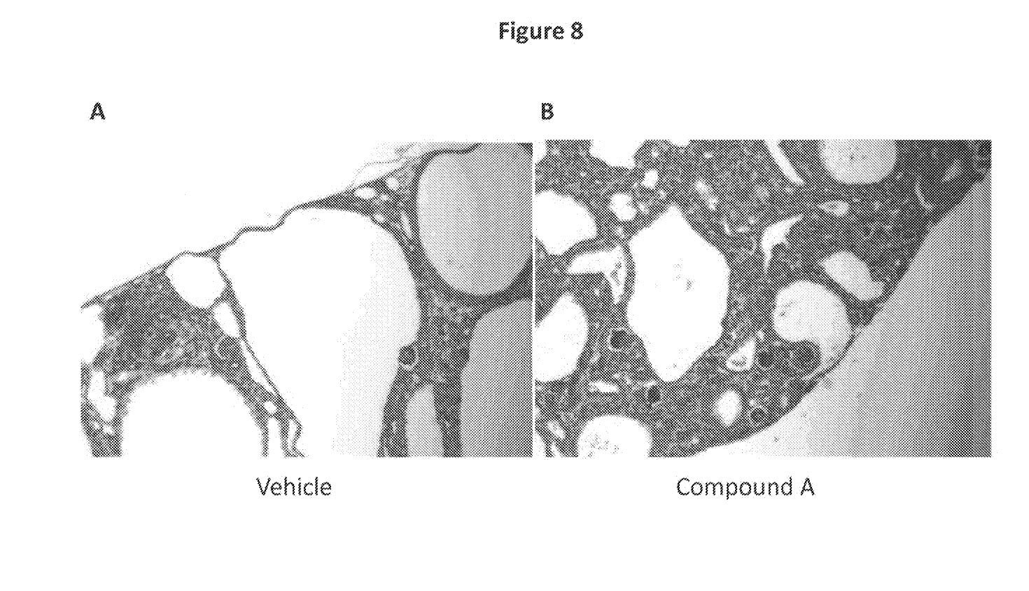

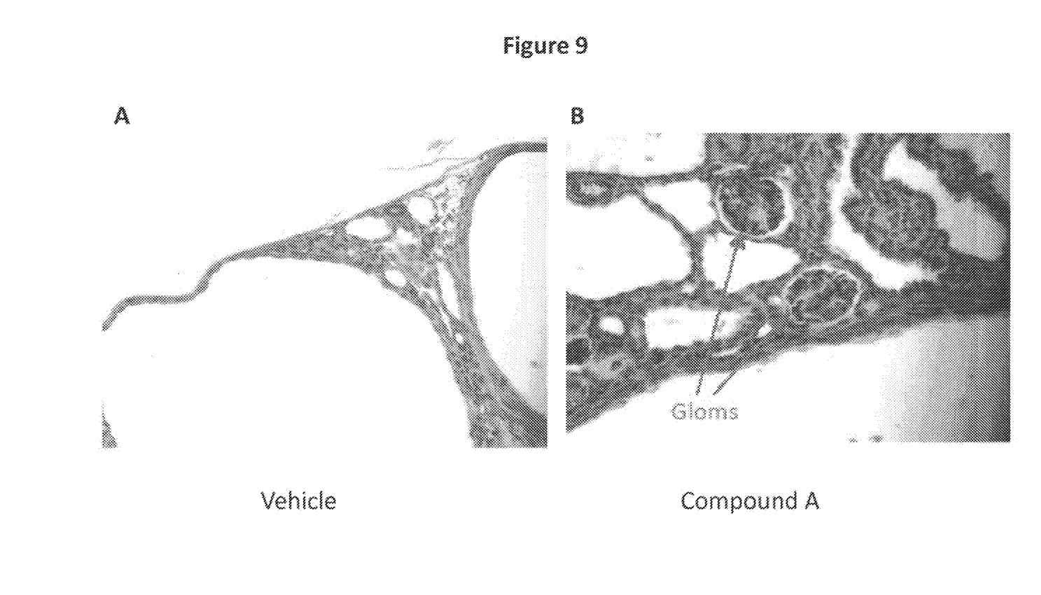

Sagittal sections of the left kidney from mice treated with vehicle or Compound A were fixed, embedded in paraffin, and sectioned for histological analysis as described in Piontek K B, et. al, (2004) J Am Soc Nephrol 15:3035-33043. Sections were stained with H&E at observed with a microscope at 4×, 100×, 20×, and 40× magnification. (FIGS. 6-9) Cyst volume was lower and parenchyma was increased in Compound A-treated mice, quantitated using ImageJ software. The number of glomeruli was increased in the Compound A treated section as compared to the vehicle treated section. (FIG. 8) Upon closer observation (40× magnification), the glomeruli appeared normal in the Compound A treated kidney as compared to the untreated kidney. (FIG. 9)

In summary, treatment with mTORC1/2 inhibitors, in particular a compound of Formula (I), in V/V mice (i) blocked mTOR1/2 signaling, and (ii) decreased renal mass and (iii) inhibited cyst formation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A pharmaceutical composition comprising a compound of Formula (I) and a pharmaceutically acceptable excipient for use in a method of treating polycystic kidney disease (PKD) in a subject in need thereof, comprising administering to the subject a therapeutically effective amount of a compound of Formula (I): 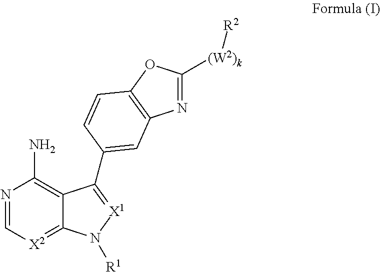
wherein:
X¹ is N or C-E¹;
X² is N or CH;
E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, or —N(R^{7A})C(O)N(R^{8A})—;
W² is —O—, —NR⁷—, —S(O)₀₋₂—, —C(O)—, —C(O)N(R⁷)—, —N(R⁷)C(O)—, or —N(R⁷)C(O)N(R⁸)—;
j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

2. The pharmaceutical composition of claim 1, wherein the compound selectively inhibits both mTORC1 and mTORC2 activity.

3. The pharmaceutical composition of claim 2, wherein the compound selectively inhibits both mTORC1 and mTORC2 activity relative to one or more type I phosphatidylinositol 3-kinases (PI3-kinase) as ascertained in a cell-based assay or an in vitro kinase assay, wherein the one or more type I PI3-kinase is selected from the group consisting of PI3-kinase α, PI3-kinase β, PI3-kinase γ, and PI3-kinase δ.

4. The pharmaceutical composition of claim 1, wherein the compound inhibits mTOR activity with an IC₅₀ value of about 100 nM or less as ascertained in an in vitro kinase assay.

5. The pharmaceutical composition of claim 1, wherein the compound inhibits mTOR activity with an IC₅₀ value of about 10 nM or less as ascertained in an in vitro kinase assay.

6. The pharmaceutical composition of claim 1, wherein the compound decreases kidney size, decreases cyst volume, and/or increases glomeruli number in a subject.

7. The pharmaceutical composition of claim 1, wherein the compound is administered parenterally, orally, intraperitoneally, intravenously, intraarterially, transdermally, intramuscularly, liposomally, via local delivery by catheter or stent, subcutaneously, intraadiposally, or intrathecally.

8. The pharmaceutical composition of claim 1, wherein said treatment reduces kidney mass in a subject by at least 10%.

9. The pharmaceutical composition of claim 1, wherein said treatment reduces kidney mass in a subject by at least 50%.

10. The pharmaceutical composition of claim 1, wherein said treatment reduces normalized kidney mass in a subject by at least 10%.

11. The pharmaceutical composition of claim 1, wherein said treatment reduces normalized kidney mass in a subject by at least 30%.

12. The pharmaceutical composition of claim 1, wherein administration of the compound is prior to, concurrent with, or after administration of another treatment to a subject.

13. A pharmaceutical composition comprising a compound of Formula (I) and a pharmaceutically acceptable excipient for use in a method of inhibiting cyst formation in a subject at risk for developing PKD, comprising contacting cyst cells with a compound of Formula (I) in an amount sufficient to inhibit growth of cyst cells: 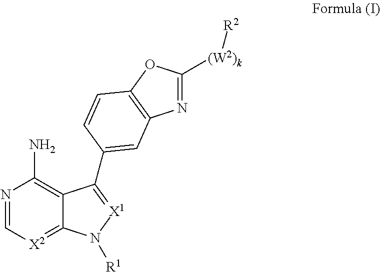
wherein:
X¹ is N or C-E¹;
X² is N or CH;
E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, or —N(R^{7A})C(O)N(R^{8A})—;
W² is —O—, —NR⁷—, —S(O)₀₋₂—, —C(O)—, —C(O)N(R⁷)—, —N(R⁷)C(O)—, or —N(R⁷)C(O)N(R⁸)—;
j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

14. The pharmaceutical composition of claim 13, for use in a method of treatment further comprising reducing cyst formation in an organ other than kidney.

15. A pharmaceutical composition comprising a compound of Formula (I) and a pharmaceutically acceptable excipient for use in a method of treatment of PKD comprising:
evaluating whether a subject is susceptible to PKD, wherein said evaluation comprises testing for (i) the presence of a biomarker correlated with PKD in said subject; and/or (ii) the presence of multiple kidney cysts; and
administering the pharmaceutical composition to the subject being tested for (a)(i) and/or (a)(ii): 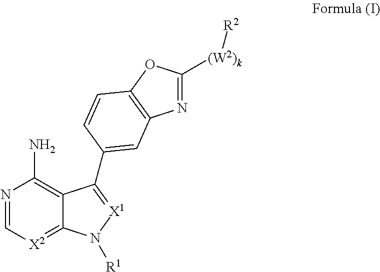
wherein:
X¹ is N or C-E¹;
X² is N or CH;
E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, or —N(R^{7A})C(O)N(R^{8A})—;
W² is —O—, —NR⁷—, —S(O)₀₋₂—, —C(O)—, —C(O)N(R⁷)—, —N(R⁷)C(O)—, or —N(R⁷)C(O)N(R⁸)—;
j is 0 or 1;
k is 0 or 1;
R¹ is —H, —C₁₋₁₀alkyl, —C₃₋₈cycloalkyl, —C₁₋₁₀alkyl-C₃₋₈cycloalkyl, or heterocyclyl, each of which is unsubstituted or is substituted by one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², bicyclic aryl, substituted monocyclic aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₂₋₁₀alkyl-monocyclic aryl, monocyclic aryl-C₂₋₁₀alkyl, C₁₋₁₀alkylbicycloaryl, bicycloaryl-C₁₋₁₀alkyl, substituted C₁₋₁₀alkylaryl, substituted aryl-C₁₋₁₀alkyl, C₁₋₁₀alkylheteroaryl, C₁₋₁₀alkylheterocyclyl, C₂₋₁₀alkenyl, C₂₋₁₀alkynyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkenyl-C₃₋₈cycloalkyl, C₂₋₁₀alkynyl-C₃₋₈cycloalkenyl, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, heterocyclyl C₁₋₁₀alkyl, heterocyclylC₂₋₁₀alkenyl, heterocyclyl-C₂₋₁₀alkynyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroaryl-heteroalkyl, or heteroaryl-heterocyclyl, wherein each of said bicyclic aryl, monocyclic aryl, or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or is substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
R³ and R⁴ are independently hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₁₋₁₀alkylaryl, C₁₋₁₀alkylheteroaryl, C₁₋₁₀alkylheterocyclyl, C₂₋₁₀alkenyl, C₂₋₁₀alkynyl, C₂₋₁₀alkenyl-C₁₋₁₀alkyl, C₂₋₁₀alkynyl-C₁₋₁₀alkyl, C₂₋₁₀alkenylaryl, C₂₋₁₀alkenylheteroaryl, C₂₋₁₀alkenylheteroalkyl, C₂₋₁₀alkenylheterocyclyl, C₂₋₁₀alkenyl-C₃₋₈cycloalkyl, C₂₋₁₀alkynyl-C₃₋₈cycloalkyl, C₂₋₁₀alkynylaryl, C₂₋₁₀alkynylheteroaryl, C₂₋₁₀alkynylheteroalkyl, C₂₋₁₀alkynylheterocyclyl, C₂₋₁₀alkynyl-C₃₋₈cycloalkenyl, C₁₋₁₀alkoxy C₁₋₁₀alkyl, C₁₋₁₀alkoxy-C₂₋₁₀alkenyl, C₁₋₁₀alkoxy-C₂₋₁₀alkynyl, heterocyclyl, heterocyclyl-C₁₋₁₀alkyl, heterocyclyl-C₂₋₁₀alkenyl, heterocyclyl-C₂₋₁₀alkynyl, aryl-C₁₋₁₀alkyl, aryl-C₂₋₁₀alkenyl, aryl-C₂₋₁₀alkynyl, aryl-heterocyclyl, heteroaryl-C₁₋₁₀alkyl, heteroaryl-C₂₋₁₀alkenyl, heteroaryl-C₂₋₁₀alkynyl, heteroaryl-C₃₋₈cycloalkyl, heteroalkyl, heteroaryl-heteroalkyl, or heteroaryl-heterocyclyl, wherein each of said aryl or heteroaryl moiety is unsubstituted or is substituted with one or more independent halo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³², and wherein each of said alkyl, cycloalkyl, heterocyclyl, or heteroalkyl moiety is unsubstituted or substituted with one or more halo, —OH, —R³¹, —CF₃, —OCF₃, —OR³¹, —O-aryl, —NR³¹R³², —NR³⁴R³⁵, —C(O)R³¹, —CO₂R³¹, —C(═O)NR³⁴R³⁵, or —C(═O)NR³¹R³²;
each of R³¹, R³², and R³³ is independently H or C₁₋₁₀alkyl, wherein the C₁₋₁₀alkyl is unsubstituted or is substituted with one or more aryl, heteroalkyl, heterocyclyl, or heteroaryl substituent, wherein each of said aryl, heteroalkyl, heterocyclyl, or heteroaryl substituent is unsubstituted or is substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl) or —SO₂NR³⁴R³⁵;
R³⁴ and R³⁵ in —NR³⁴R³⁵, —C(═O)NR³⁴R³⁵, or —SO₂NR³⁴R³⁵, are independently taken together with the nitrogen atom to which they are attached to form a 3-10 membered saturated or unsaturated ring; wherein said ring is independently unsubstituted or is substituted by one or more —NR³¹R³², hydroxyl, halogen, oxo, aryl, heteroaryl, C₁₋₆alkyl, or O-aryl, and wherein said 3-10 membered saturated or unsaturated ring independently contains 0, 1, or 2 more heteroatoms in addition to the nitrogen atom;
each of R⁷, R^{7A}, R⁸, and R^{8A} is independently hydrogen, C₁₋₁₀alkyl, C₂₋₁₀alkenyl, aryl, heteroaryl, heterocyclyl or C₃₋₁₀cycloalkyl, each of which except for hydrogen is unsubstituted or is substituted by one or more independent R⁶ substituents; and
R⁶ is independently halo, —OR³¹, —SH, NH₂, —NR³⁴R³⁵, —NR³¹R³², —CO₂R³¹, —CO₂aryl, —C(═O)NR³¹R³², C(═O)NR³⁴R³⁵, —NO₂, —CN, —S(O)₀₋₂C₁₋₁₀alkyl, —S(O)₀₋₂aryl, —SO₂NR³⁴R³⁵, —SO₂NR³¹R³², C₁₋₁₀alkyl, C₂₋₁₀alkenyl, C₂₋₁₀alkynyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl, each of which is unsubstituted or is substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

16. The pharmaceutical composition of claim 15, wherein the biomarker is a mutated PKD-1 or PKD-2 gene, or a respective gene product.

17. A pharmaceutical composition comprising a compound of Formula (I) and a pharmaceutically acceptable excipient for use in a method of treating a polycystic disease in a subject in need thereof, comprising administering to the subject a therapeutically effective amount of a compound of Formula (I): 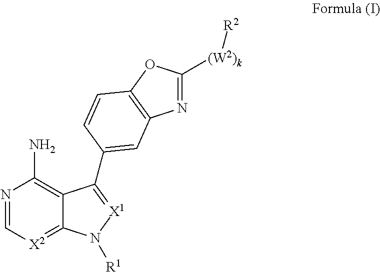
wherein:
X¹ is N or C-E¹;
X² is N or CH;
E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is —H, -aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R¹ is unsubstituted or substituted with one or more independent R³;
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR31R32, aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein R² is unsubstituted or is substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —C(═O)NR³¹R³², —OC(═O)SR³¹, —SC(═O)OR³¹, —P(O)OR³¹OR³², —O-aryl or —SC(═O)NR³¹R³²;
R³ and R⁴ are independently hydrogen, halogen, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², aryl, heteroaryl, heterocylcyl, C₁₋₁₀alkyl, C₃₋₈cycloalkyl, C₁₋₁₀alkyl-C₃₋₈cycloalkyl, C₃₋₈cycloalkyl-C₁₋₁₀alkyl, C₃₋₈cycloalkyl-C₂₋₁₀alkenyl, C₃₋₈cycloalkyl-C₂₋₁₀alkynyl, C₁₋₁₀alkyl-C₂₋₁₀alkenyl, C₁₋₁₀alkyl-C₂₋₁₀alkynyl, C₂₋₁₀alkenyl-C₁₋₁₀alkyl, C₂₋₁₀alkynyl-C₁₋₁₀alkyl, C₁₋₁₀alkylaryl, arylC₁₋₁₀alkyl, C₁₋₁₀alkylheteroaryl, heteroaryl-C₁₋₁₀alkyl, C₁₋₁₀alkylheteroalkyl, heteroalkylC₁₋₁₀alkyl, C₁₋₁₀alkylheterocyclyl, heterocyclyl C₁₋₁₀alkyl, C₂₋₁₀alkenyl, C₂₋₁₀alkenylC₂₋₁₀alkynyl, C₂₋₁₀alkynylC₂₋₁₀alkenyl, C₂₋₁₀alkenyl-C₃₋₈cycloalkyl, C₃₋₈cycloalkylC₂₋₁₀alkenyl, C₂₋₁₀alkenylaryl, aryl-C₂₋₁₀alkenyl, C₂₋₁₀alkenylheteroaryl, heteroaryl-C₂₋₁₀alkenyl, C₂₋₁₀alkenylheteroalkyl, heteroalkylC₂₋₁₀alkenyl, C₂₋₁₀alkenylheterocyclyl, heterocyclylC₂₋₁₀alkenyl, C₂₋₁₀alkynyl, C₂₋₁₀alkynyl-C₃₋₈cycloalkyl, C₃₋₈cycloalkylC₂₋₁₀alkynyl, C₂₋₁₀alkynylaryl, aryl-C₂₋₁₀alkynyl, C₂₋₁₀alkynylheteroaryl, heteroaryl-C₂₋₁₀alkynyl, C₂₋₁₀alkynylheteroalkyl, heteroalkylC₂₋₁₀alkynyl, C₂₋₁₀alkynylheterocyclyl, heterocyclyl-C₂₋₁₀alkynyl, C₁₋₁₀alkoxy, C₁₋₁₀alkoxy C₁₋₁₀alkyl, C₁₋₁₀alkoxyC₂₋₁₀alkenyl, C₁₋₁₀alkoxyC₂₋₁₀alkynyl, heterocyclyl, aryl-heterocyclyl, heteroaryl-heterocyclyl, heterocyclyl-aryl, heterocyclyl-heteroaryl, heterocycly-C₃₋₈cycloalkyl, C₃₋₈cycloalkyl-heterocyclyl, heteroalkyl, heteroalkylC₃₋₈cycloalkyl, C₃₋₈cycloalkyl-heteroalkyl, heteroalkyl-heterocyclyl, heterocyclyl-heteroalkyl, heteroalkyl-aryl, aryl-heteroalkyl, heteroalkyl-heteroaryl, heteroaryl-heteroalkyl, C₃₋₈cycloalkyl-aryl, aryl-C₃₋₈cycloalkyl, C₃₋₈cycloalkyl-heteroaryl, heteroaryl-C₃₋₈cycloalkyl, aryl-heteroaryl, heteroaryl-aryl, monocyclic aryl-C₁₋₁₀alkyl, C₁₋₁₀alkyl-monocyclic aryl, bicycloaryl-C₁₋₁₀alkyl, C₁₋₁₀alkyl-bicycloaryl, C₃₋₈cycloalkenyl, C₁₋₁₀alkyl-C₃₋₈cycloalkenyl, C₃₋₈cycloalkenyl-C₁₋₁₀alkyl, C₃₋₈cycloalkenyl-C₂₋₁₀alkenyl, C₂₋₁₀alkenyl-C₃₋₈cycloalkenyl, C₃₋₈cycloalkenyl-C₂₋₁₀alkynyl, C₂₋₁₀alkynyl-C₃₋₈cycloalkenyl, C₃₋₈cycloalkenyl-heteroalkyl, heteroalkyl-C₃₋₈cycloalkenyl, C₃₋₈cycloalkyl-C₃₋₈cycloalkenyl, C₃₋₈cycloalkenyl-C₃₋₈cycloalkyl, C₃₋₈cycloalkenylaryl, aryl C₃₋₈cycloalkenyl, C₃₋₈cycloalkenylheteroaryl, heteroaryl C₃₋₈cycloalkenyl, C₃₋₈cycloalkenylheterocyclyl, heterocyclyl C₃₋₈cycloalkenyl, C₃₋₈cycloalkynyl, C₁₋₁₀alkyl-C₃₋₈cycloalkynyl, C₃₋₈cycloalkynyl-C₁₋₁₀alkyl, C₃₋₈cycloalkynyl-C₂₋₁₀alkenyl, C₂₋₁₀alkenyl-C₃₋₈cycloalkynyl, C₃₋₈cycloalkynyl-C₂₋₁₀alkynyl, C₂₋₁₀alkynyl-C₃₋₈cycloalkynyl, C₃₋₈cycloalkynyl-heteroalkyl, heteroalkyl-C₃₋₈cycloalkynyl, C₃₋₈cycloalkenyl-C₃₋₈cycloalkynyl, C₃₋₈cycloalkynyl-C₃₋₈cycloalkenyl, C₃₋₈cycloalkyl-C₃₋₈cycloalkynyl, C₃₋₈cycloalkynyl-C₃₋₈cycloalkyl, C₃₋₈cycloalkynylaryl, aryl C₃₋₈cycloalkynyl, C₃₋₈cycloalkynylheteroaryl, heteroaryl C₃₋₈cycloalkynyl, C₃₋₈cycloalkynylheterocyclyl, heterocyclyl C₃₋₈cycloalkynyl, substituted C₁₋₁₀alkylaryl, substituted aryl-C₁₋₁₀alkyl, or C₂₋₁₀alkynyl-C₃₋₈cycloalkenyl;
wherein each R³ and R⁴ is independently unsubstituted or substituted with one or more independent halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², or —SC(═O)NR³¹R³²;
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or C₁₋₁₀alkyl, C₂₋₁₀alkenyl, C₂₋₁₀alkynyl, C₃₋₈cycloalkyl, heteroalkyl, aryl, heteroaryl, or heterocyclyl moiety, wherein each of said moieties is unsubstituted or is substituted with one or more C₁₋₁₀alkyl, C₂₋₁₀alkenyl, C₂₋₁₀alkynyl, C₃₋₁₀cycloalkyl, heteroalkyl, aryl, heteroaryl, or heterocyclyl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R⁷, R^{7A}, R⁸, and R^{8A} is independently hydrogen, C₁₋₁₀alkyl, C₂₋₁₀alkenyl, aryl, heteroalkyl, heteroaryl, heterocyclyl or C₃₋₁₀cycloalkyl, each of which except for hydrogen is unsubstituted or is substituted by one or more independent R⁶ substituents; and
R⁶ is independently halo, oxo, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³²; or C₁₋₁₀alkyl, C₂₋₁₀alkenyl, C₂₋₁₀alkynyl, C₃₋₈cycloalkyl, heteroalkyl, aryl, heteroaryl, heterocyclyl, aryl-C₁₋₁₀alkyl, aryl-C₂₋₁₀alkenyl, aryl-C₂₋₁₀alkynyl, heteroaryl-C₁₋₁₀alkyl, heteroaryl-C₂₋₁₀alkenyl, or heteroaryl-C₂₋₁₀alkynyl,
wherein each R⁶ is independently unsubstituted or substituted with one or more independent halo, oxo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR³¹R³², —C(═O)NR³⁴R³⁵, —SO₂NR³⁴R³⁵, —SO₂NR³¹R³², —NR³¹R³², or —NR³⁴R³⁵.

18. The pharmaceutical composition of claim 17, wherein said polycystic disease is polycystic kidney disease.

19. A pharmaceutical composition comprising a compound of Formula (I) and a pharmaceutically acceptable excipient for use in a method of treating a polycystic disease in a subject in need thereof, comprising administering to the subject a therapeutically effective amount of a compound of Formula (I): 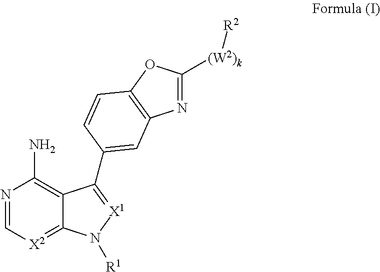
wherein:
X¹ is N or C-E¹;
X² is N or CH;
E¹ is —(W¹)ⱼ—R⁴;
W¹ is —O—, —NR^{7A}—, —S(O)₀₋₂—, —C(O)—, —C(O)N(R^{7A})—, —N(R^{7A})C(O)—, —N(R^{7A})S(O)—, —N(R^{7A})S(O)₂—, —C(O)O—, —CH(R^{7A})N(C(O)OR^{8A})—, —CH(R^{7A})N(C(O)R^{8A})—, —CH(R^{7A})N(SO₂R^{8A})—, —CH(R^{7A})N(R^{8A})—, —CH(R^{7A})C(O)N(R^{8A})—, —CH(R^{7A})N(R^{8A})C(O)—, —CH(R^{7A})N(R^{8A})S(O)—, or —CH(R^{7A})N(R^{8A})S(O)₂—;
W² is —O—, —NR⁷—, —S(O)₀₋₂—, —C(O)—, —C(O)N(R⁷)—, —N(R⁷)C(O)—, —N(R⁷)S(O)—, —N(R⁷)S(O)₂—, —C(O)O—, —CH(R⁷)N(C(O)OR⁸)—, —CH(R⁷)N(C(O)R⁸)—, —CH(R⁷)N(SO₂R⁸)—, —CH(R⁷)N(R⁸)—, —CH(R⁷)C(O)N(R⁸)—, —CH(R⁷)N(R⁸)C(O)—, —CH(R⁷)N(R⁸)S(O)—, or —CH(R⁷)N(R⁸)S(O)₂— or —N(R⁷)C(O)N(R⁸)—;
j is 0 or 1;
k is 0 or 1;
R¹ is hydrogen, R³-substituted or unsubstituted C₁₋₁₀alkyl, R³-substituted or unsubstituted C₂₋₁₀alkenyl, R³-substituted or unsubstituted C₂₋₁₀alkynyl, R³-substituted or unsubstituted C₃₋₈cycloalkyl, R³-substituted or unsubstituted C₃₋₈cycloalkenyl, R³-substituted or unsubstituted C₃₋₈cycloalkynyl, R³-substituted or unsubstituted heteroalkyl, R³-substituted or unsubstituted heteroalkenyl, R³-substituted or unsubstituted heteroalkynyl, R³-substituted or unsubstituted heterocyclyl, R³-substituted or unsubstituted aryl, R³-substituted or unsubstituted heteroaryl; wherein each R³-substituted R¹ is independently substituted with one or more R³
R² is hydrogen, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R² is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).
R³ and R⁴ are independently is hydrogen, oxo, halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R³ or R⁴ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl);
R³¹, R³², and R³³ in each instance is independently H, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵; or substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each R³¹, R³², and R³³ in each instance is independently unsubstituted or is substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R³⁴ and R³⁵ together with the nitrogen atom to which they are attached independently form a 3-10 membered saturated or unsaturated ring containing 1-3 heteroatoms; wherein said ring is independently unsubstituted or substituted with one or more oxo, aryl, heteroaryl, halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —NR³⁴R³⁵, —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NR³⁴R³⁵, —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), —SO₂NH(C₁₋₁₀alkyl), —COOH, or —SO₂NR³⁴R³⁵;
each R⁷, R^{7A}, R⁸, and R^{8A} is independently hydrogen, R⁶-substituted or unsubstituted C₁₋₁₀alkyl, R⁶-substituted or unsubstituted C₂₋₁₀alkenyl, R⁶-substituted or unsubstituted C₂₋₁₀alkynyl, R⁶-substituted or unsubstituted C₃₋₈cycloalkyl, R⁶-substituted or unsubstituted C₃₋₈cycloalkenyl, R⁶-substituted or unsubstituted C₃₋₈cycloalkynyl, R⁶-substituted or unsubstituted heteroalkyl, R⁶-substituted or unsubstituted heteroalkenyl, R⁶-substituted or unsubstituted heteroalkynyl, R⁶-substituted or unsubstituted heterocyclyl, R⁶-substituted or unsubstituted aryl, R⁶-substituted or unsubstituted heteroaryl; wherein each R⁶-substituted R⁷, R^{7A}, R⁸ and R^{8A} is independently substituted with one or more R⁶; and
R⁶ is independently halogen, —OH, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl;
wherein each substituted R⁶ is independently substituted with one or more independent halogen, —OH, oxo, —R³¹, —CF₃, —OCF₃, —OR³¹, —NR³¹R³², —NR³⁴R³⁵, —C(O)R³¹, —CO₂R³¹, —C(═O)NR³¹R³², —C(═O)NR³⁴R³⁵, —NO₂, —CN, —S(O)₀₋₂R³¹, —SO₂NR³¹R³², —SO₂NR³⁴R³⁵, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³², —NR³¹C(═NR³²)OR³³, —NR³¹C(═NR³²)SR³³, —OC(═O)OR³³, —OC(═O)NR³¹R³², —OC(═O)SR³¹, —SC(═O)OR³¹, —P(O)OR³¹OR³², —SC(═O)NR³¹R³², substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈cycloalkenyl, substituted or unsubstituted C₃₋₈cycloalkynyl, substituted or unsubstituted heteroalkyl, substituted or unsubstituted heteroalkenyl, substituted or unsubstituted heteroalkynyl, substituted or unsubstituted heterocyclyl, substituted or unsubstituted aryl, substituted or unsubstituted heteroaryl; wherein each such substituted moiety is independently substituted with one or more halo, oxo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl).
